(19) United States
(12) Patent Application Publication
(10) Pub. No.: US 2025/0388968 A1
(43) Pub. Date: Dec. 25, 2025

(54) DETECTION OF CELL DAMAGE

(71) Applicant: Garvan Institute of Medical Research, Darlinghurst (AU)

(72) Inventors: David Hugh MURRAY, Darlinghurst (AU); Nicola Rosalind BOULTER, Darlinghurst (AU)

(21) Appl. No.: 18/877,805

(22) PCT Filed: Jun. 21, 2023
(86) PCT No.: PCT/AU2023/050562

§ 371 Date: Dec. 20, 2024

(30) Foreign Application Priority Data
Jun. 21, 2022 (AU) 2022901697

(51) Int. Cl. C12Q 1/6883

(52) U.S. Cl. CPC C12Q 1/6883; C12Q 2600/154

(57) ABSTRACT

Epigenetic modifications play an important role in regulating cell-specific expression patterns. Different DNA methylation signatures, for example, can be found in different tissues and even between different cell types within a particular tissue. In work leading to the present invention, the inventors found that these methylation signatures can be used to identify cfDNA tissue of origin. Moreover, these novel methylation markers can be used to detect cell, tissue or organ damage, including autologous cell, tissue or organ damage.

### FIELD OF THE DISCLOSURE

The present disclosure relates to methods and compositions for detecting cell, tissue and organ damage using cell free DNA.

### BACKGROUND OF THE DISCLOSURE

Any discussion of the prior art throughout the specification should in no way be considered as an admission that such prior art is widely known or forms part of the common general knowledge in the field.

Cell free DNA (cfDNA) in the blood of healthy individuals primarily comes from white blood cells, with 20-30% contributed by organs across the body from normal cell turnover. During injury and disease, organ- or tissue-specific cell death results in an increase in DNA contribution by that organ or tissue to the cfDNA population. Detection of these changes in cfDNA levels has been applied to monitoring graft rejection in organ transplants by measuring donor-derived cell free DNA (dd-cfDNA) found in the transplant recipient.

Organ-specific cfDNA detection has been achieved using Y-chromosomal markers in female patients receiving an organ from a male donor. More recently, massively parallel sequencing methods have been used to identify donor-specific alleles or single-nucleotide polymorphisms (SNPs). However, dd-cfDNA assays are only applicable to situations where chimerism exists, such as in organ transplant recipients.

There is a need for methods and compositions for detecting and monitoring tissue- or organ-specific cfDNA from native/autologous organs.

### SUMMARY OF THE DISCLOSURE

Epigenetic modifications play an important role in regulating cell-specific expression patterns. Different DNA methylation signatures, for example, can be found in different tissues and even between different cell types within a particular tissue. In work leading to the present invention, the inventors found that these epigenetic signatures can be used to identify cfDNA tissue of origin. Moreover, these novel epigenetic markers can be used to detect cell, tissue or organ damage, including autologous cell, tissue or organ damage.

In one aspect, the present disclosure provides a method of diagnosing organ damage in a subject the method comprising detecting an organ-specific epigenetic marker in cfDNA obtained from a biological sample of the subject, wherein the presence of the epigenetic marker in the cfDNA is indicative of organ damage.

In another aspect, the present disclosure provides a method of detecting organ damage in a subject, the method comprising: a) obtaining a biological sample comprising cfDNA from the subject; and b) detecting an organ-specific epigenetic marker in the cfDNA of the sample, wherein the presence of the epigenetic marker in the cfDNA of the sample is indicative of organ damage.

In yet a further aspect, present disclosure provides a method of identifying at least one methylated region in cfDNA, said method comprising the steps of:
(i) obtaining cfDNA from a subject;
(ii) treating the cfDNA with bisulfite to obtain bisulfite converted cfDNA; and
(iii) identifying the at least one methylated region by PCR amplification of the bisulfite converted cfDNA with primers that selectively amplify the at least one methylated region,
   wherein the at least one methylated region is a differentially methylated region that occurs in kidney cells

In some examples, the method comprises monitoring kidney damage during renal replacement therapy.

In some examples, the method comprises detecting an increase in the level of the epigenetic marker relative to a reference level. In some examples, the method comprises detecting an increase in the level of the epigenetic marker over time.

The epigenetic marker is preferably DNA methylation status at a differentially methylated region of the cfDNA.

In some examples, the method comprises detecting cfDNA methylation status at more than one differentially methylated region. The methylation status may be determined at more than one differentially methylated region using a multiplex assay.

In some examples, the methylation status is determined by a method that does not involve genomic DNA sequencing. The methylation status is preferably determined by a method that does not involve DNA sequencing. The methylation status may, for example, be determined by treating the cfDNA with bisulfite and amplifying the differentially methylated region using polymerase chain reaction (PCR). The PCR may be digital PCR (dPCR), droplet digital PCR (ddPCR) or quantitative PCR (qPCR).

In some examples, the organ is a kidney. The organ damage may be associated with acute kidney injury (AKI), chronic kidney disease (CKD) or kidney transplant rejection following organ donation. In some examples, the organ damage is associated with chemotherapy or radiotherapy. The biological sample may be saliva, blood or serum or plasma, urine, semen, vitreous fluid, lymph fluid, synovial fluid, follicular fluid, gastric fluid, intestinal fluid, bile, tumour fluid, interstitial fluid, amniotic fluid, mucus, breast milk, pleural fluid, sweat, tears, stool, serum or cerebro-spinal fluid.

In a further aspect, the present disclosure provides a method of diagnosing kidney damage in a subject, the method comprising detecting at least one kidney differentially methylated region in cfDNA wherein the cfDNA is obtained from a biological sample of the subject, and wherein the presence of the at least one kidney differentially methylated region in the cfDNA is indicative of kidney damage.

In still a further aspect, the present disclosure provides a method of detecting kidney damage in a subject, the method comprising:
a) obtaining a biological sample comprising cfDNA from the subject; and
b) detecting at least one kidney differentially methylated region in the cfDNA,
   wherein the presence of the at least one kidney-specific methylation site in the cfDNA is indicative of organ damage.

In some examples, method comprises detecting an increase in the level of the at least one kidney differentially methylated region relative to a reference level. In further examples, the method comprises detecting an increase in the level of the at least one kidney differentially methylated region over time. In still further examples, the method comprises detecting cfDNA methylation status at more than one kidney differentially methylated region. In yet further examples of the method, the methylation status is determined at more than one kidney differentially methylated region using a multiplex assay. In certain examples of the method, the methylation status is determined by a method that does not involve DNA sequencing. In certain examples of the method, the methylation status is determined by treating the cfDNA with bisulfite and amplifying the at least one kidney differentially methylated region using polymerase chain reaction (PCR), where the PCR is, for example, digital PCR (dPCR), digital droplet PCR (ddPCR) or quantitative PCR (qPCR).

In particular examples of the method, the subject and the kidney are autologous. In further examples of the method, the kidney damage is associated with acute kidney injury, chronic kidney disease or kidney transplant rejection or Renal replacement therapy. In further examples of the method, the kidney damage is associated with chemotherapy or radiotherapy. In still further examples of the method, the biological sample is urine.

In particular examples of the method, the method specifically detects damage to a defined tissue or cell-type of the kidney. For example, damage to renal proximal tubule epithelial cells, or damage to podocytes.

In certain examples the subject is a human. In other examples, the subject is non-human. For example, in certain examples the non-human subject is, for example, a domesticated animal or a companion animal, where a domesticated animal can for example, be selected from the group consisting of sheep, cattle, horses, cats, dogs, pigs, and chickens and a companion animal can be selected from, for example, cats and dogs.

In a particular example, the method further comprises treating the subject for the kidney damage.

In some examples, the differentially methylated region is located at one or more loci selected from the group consisting of GRAMD1B, DDC, MAST4, MCF2L, PAX2, chr12-122277360 (CLIP 1), chr17-35303285, DEF6, EMX1, HPD, PDE4D and SPAG5. In some examples, the differentially methylated region is located at one or more loci selected from the group consisting of GRAMD1B, DDC, MAST4, MCF2L and PAX2. In some examples, the differentially methylated regions are located at two loci selected from the group consisting of GRAMD1B, DDC, MAST4, MCF2L and PAX2. For example, the differentially methylated regions may be located at: GRAMD1B and DDC; GRAMD1B and MAST4; GRAMD1B and MCF2L; GRAMD1B and PAX2; DDC and MAST4; DDC and MCF2L; DDC and PAX2; MAST4 and MCF2L; MAST4 and PAX2; or MCF2L and PAX2. In some examples, the differentially methylated regions are located at three loci selected from the group consisting of GRAMD1B, DDC, MAST4, MCF2L and PAX2. For example, the differentially methylated regions may be located at: GRAMD1B, DDC and MAST4; GRAMD1B, DDC and MCF2L; GRAMD1B, DDC and PAX2; GRAMD1B, MAST4 and MCF2L; GRAMD1B, MAST4 and PAX2; GRAMD1B, MCF2L and PAX2; DDC, MAST4 and MCF2L; DDC, MAST4 and PAX2; DDC, MCF2L and PAX2; or MAST4; MCF2L and PAX2. In some examples, the differentially methylated regions are located at four loci selected from the group consisting of GRAMD1B, DDC, MAST4, MCF2L and PAX2. For example, the differentially methylated regions may be located at GRAMD1B, DDC, MAST4 and MCF2L; GRAMD1B, DDC, MAST4 and PAX2; GRAMD1B, MAST4, MCF2L and PAX2; GRAMD1B, DDC, MCF2L and PAX2; or DDC, MAST4, MCF2L and PAX2. In some examples, the differentially methylated regions are located at GRAMD1B, DDC, MAST4, MCF2L and PAX2. The differentially methylated region may comprise a sequence having at least 90% identity to any one or more of SEQ ID NO. 1, SEQ ID NO. 2 SEQ ID NO. 8, SEQ ID NO. 9, SEQ ID NO. 15, SEQ ID NO. 16, SEQ ID NO. 22, SEQ ID NO. 23, SEQ ID NO. 29 or SEQ ID NO. 30.

It will be understood that tissue damage may include damage to a specific cell- or tissue-type within the tissue. In some examples, the method specifically detects damage to a defined tissue or cell-type of the organ. The defined cell-type may be renal proximal tubule epithelial cells. The differentially methylated regions may be located at at least one of GRAMD1B, DDC, MAST4, MCF2L, PAX2, chr12-122277360 (CLIP 1) chr17-35303285, DEF6, EMX1, HPD, PDE4D and SPAG5.

In some examples, the method further comprises treating the subject for the organ damage. For example, the present disclosure also provides a method of treating organ damage in a subject, the method comprising: i) detecting an organ-specific epigenetic marker in cfDNA obtained from a biological sample of the subject, wherein the presence of the epigenetic marker in the cfDNA is indicative of organ damage; and ii) treating the subject for the organ damage.

In another aspect, the present disclosure provides a method of indicating to a user whether or not a subject has organ damage, the method comprising:
a) producing sample epigenetic data by determining a level of an organ-specific epigenetic marker in cell-free DNA obtained from a biological sample of the subject;
b) a processor receiving the sample epigenetic data, wherein the processor also receives reference epigenetic data corresponding to the epigenetic marker;
c) the processor generating differential epigenetic data by comparing the sample epigenetic data with the reference epigenetic data;
d) the processor processing the differential epigenetic data to produce a damage index value;
e) determining by the processor a damage status of the subject based upon the damage index value, the damage status being indicative of whether or not the subject has organ damage; and
f) transferring an indication of the disease status of the subject to the user via a communications network.

In certain embodiments, the method of the invention relates to a companion diagnostic that is used in conjunction with other diagnostic markers and/or reference data or a subject's details to determine or predict kidney damage in the subject. Other diagnostic markers can include but are not limited to elevated blood and/or urine creatinine levels, elevated blood urea nitrogen (BUN), glomerular filtration levels, urine albumin: creatinine ratio and hyperlipidemia. Reference data or a subject's details can include but are not limited to the subject's age, weight, alcohol intake, smoking status, intake of drugs or medication regime, physical fitness or lack thereof, blood pressure, existing or susceptibility to a disease, stress or mental illness, cardiovascular disease and stroke.

The epigenetic marker is preferably DNA methylation status at a differentially methylated region within the cfDNA. In some examples, the differentially methylated region is located at one or more loci selected from the group consisting of GRAMD1B, DDC, MAST4, MCF2L, PAX2, chr12-122277360 (CLIP1), chr17-35303285, DEF6, EMX1, HPD, PDE4D and SPAG5. In some examples, the differentially methylated region is located at one or more loci selected from the group consisting of GRAMD1B, DDC, MAST4, MCF2L and PAX2. In some examples, the differentially methylated regions are located at two loci selected from the group consisting of GRAMD1B, DDC, MAST4, MCF2L and PAX2. For example, the differentially methylated regions may be located at: GRAMD1B and DDC; GRAMD1B and MAST4; GRAMD1B and MCF2L; GRAMD1B and PAX2; DDC and MAST4; DDC and MCF2L; DDC and PAX2; MAST4 and MCF2L; MAST4 and PAX2; or MCF2L and PAX2. In some examples, the differentially methylated regions are located at three loci selected from the group consisting of GRAMD1B, DDC, MAST4, MCF2L and PAX2. For example, the differentially methylated regions may be located at: GRAMD1B, DDC and MAST4; GRAMD1B, DDC and MCF2L; GRAMD1B, DDC and PAX2; GRAMD1B, MAST4 and MCF2L; GRAMD1B, MAST4 and PAX2; GRAMD1B, MCF2L and PAX2; DDC, MAST4 and MCF2L; DDC, MAST4 and PAX2; DDC, MCF2L and PAX2; or MAST4; MCF2L and PAX2. In some examples, the differentially methylated regions are located at four loci selected from the group consisting of GRAMD1B, DDC, MAST4, MCF2L and PAX2. For example, the differentially methylated regions may be located at GRAMD1B, DDC, MAST4 and MCF2L; GRAMD1B, DDC, MAST4 and PAX2; GRAMD1B, MAST4, MCF2L and PAX2; GRAMD1B, DDC, MCF2L and PAX2; or DDC, MAST4, MCF2L and PAX2. In some examples, the differentially methylated regions are located at GRAMD1B, DDC, MAST4, MCF2L and PAX2. The differentially methylated region may comprise a sequence having at least 90% identity to any one or more of SEQ ID NO. 1, SEQ ID NO. 2 SEQ ID NO. 8, SEQ ID NO. 9, SEQ ID NO. 15, SEQ ID NO. 16, SEQ ID NO. 22, SEQ ID NO. 23, SEQ ID NO. 29 or SEQ ID NO. 30.

The present invention relates to methods that use differential methylation specifically in kidney cells. In one embodiment, one of the key advantages relates to the ability to selectively amplify methylated targets sequences and exclude non-methylated versions of the genes. Thus, amplifying and identifying only kidney cfDNA. This approach not only allows for PCR-based assays but also offers an alternative low-cost sequencing-based embodiment. Accordingly, the potential of incorporating sequencing into one embodiment of the method of the invention provides a beneficial alternative pathway to overcome limitations associated with PCR alone. Nevertheless, a PCR assay of the present invention remains commercially viable due to its widespread use and relatively low expenditure required for setup.

It would be clear to the skilled person that the present invention is not limited to any particular differentially methylated region within the described loci. For example, the present invention can be carried out using at least one of a number of differentially methylated regions that occur within the described loci.

In some examples, the sample epigenetic data and the reference epigenetic data is based upon more than one epigenetic marker.

In some examples, the processor processes the differential epigenetic data using a univariate and/or multivariate analysis.

The subject may be a human or non-human subject, where a non-human subject is for example, a domesticated animal or a companion animal, where a domesticated animal can for example, be selected from the group consisting of sheep, cattle, horses, cats, dogs, pigs, and chickens and a companion animal can be selected from, for example, cats and dogs.

In some examples, the subject is a human.

In yet another aspect, the present disclosure provides at least one nucleotide primer or nucleotide probe sequence when used in the method of the invention to detect at least one kidney differentially methylated region of cfDNA. In certain examples, the at least one nucleotide primer or probe is two nucleotide primers when used in a PCR to detect a kidney differentially methylated region in cfDNA.

In yet a further aspect, the present disclosure provides a kit for use in diagnosing kidney damage in a subject comprising at least one reagent for detecting at least one kidney differentially methylated region in cfDNA wherein the cfDNA is from a biological sample of the subject including instructions for use in the method of the invention. In certain examples, the at least one reagent for detecting at least one kidney differentially methylated region in cfDNA is at least one nucleotide primer or nucleotide probe, and in certain examples, two nucleotide primers configured to detect at least one kidney differentially methylated region in cfDNA.

In yet a further aspect, the present disclosure provides a use of at least one kidney differentially methylated region in cfDNA in the manufacture of a reagent for diagnosing kidney damage in a subject. In certain examples, the reagent is at least one nucleotide primer or nucleotide probe, and in certain examples, two nucleotide primers configured to detect at least one kidney differentially methylated region in cfDNA.

### BRIEF DESCRIPTION OF THE FIGURES

FIG. 1. Candidate regions of interest plotted with the Hg19 location of each CpG probe on the x-axis and the Beta value (proportion methylated) on the y-axis. Regions of interest include DDC (A), MAST4 (B), PAX2 (C), MCF2L (D), GRAMD1B (E), chr12-122277360 (CLIP1) (F), chr17-35303285 (G), DEF6 (H), EMX1 (I), HPD (J), PDE4D (K) and SPAG5 (L). Each point in the plots represents a sample, and these are broken down into 5 categories: bulk kidney tissue, human cultured podocytes, renal cortical epithelial cells, human renal proximal tubule epithelial cells and other tissues. The lines represent the median value for each group. For regions of interest that have an assay designed, the location of the assay is highlighted with a grey box.

FIG. 2. Native sequence of PAX2 PCR assay region (A). Bisulfite converted sequence of the methylated top stand (B). Underlined sequences in (B) are the PAX2 forward primer (SEQ ID NO. 5), PAX2 reverse primer (SEQ ID NO. 6) and PAX2 oligonucleotide probe (SEQ ID NO. 7).

FIG. 3. Native sequence of GRAMD1B PCR assay region (A). Bisulfite converted sequence of the methylated top stand (B). Underlined sequences in (B) are the GRAMD1B forward primer (SEQ ID NO. 12), GRAMD1B reverse primer (SEQ ID NO. 13) and GRAMD1B oligonucleotide probe (SEQ ID NO. 14).

FIG. 4. Native sequence of DDC PCR assay region (A). Bisulfite converted sequence of the methylated top stand (B). Underlined sequences in (B) are the DDC forward primer (SEQ ID NO. 19), DDC reverse primer (SEQ ID NO. 20) and DDC oligonucleotide probe (SEQ ID NO. 21).

FIG. 5. Native sequence of MAST4 PCR assay region (A). Bisulfite converted sequence of the methylated top stand (B). Underlined sequences in (B) are the MAST4 forward primer (SEQ ID NO. 26), MAST4 reverse primer (SEQ ID NO. 27) and MAST4 oligonucleotide probe (SEQ ID NO. 28).

FIG. 6. Native sequence of MCF2L PCR assay region (A). Bisulfite converted sequence of the methylated top stand (B). Underlined sequences in (B) are the MCF2L forward primer (SEQ ID NO. 33), MCF2L reverse primer (SEQ ID NO. 34) and MCF2L oligonucleotide probe (SEQ ID NO. 35).

FIG. 7. Native sequence of ACTB PCR assay region (A). Bisulfite converted sequence of the bottom strand (B). Underlined sequences in (A) are the ACTB forward primer (SEQ ID NO. 38), ACTB reverse primer (SEQ ID NO. 39) and ACTB oligonucleotide probe (SEQ ID NO. 40). Underlined sequences in (B) are ACTB bisulfite forward primer (SEQ ID NO. 43), ACTB bisulfite reverse primer (SEQ ID NO. 44) and ACTB bisulfite oligonucleotide probe (SEQ ID NO. 45).

FIG. 8. Graph showing tissue specificity against 14 different tissue types, n=4 replicates of each at 5 ng input per replicate, and control DNA (bis UM=bisulfite converted unmethylated DNA; bis PBMC=bisulfite converted peripheral blood mononuclear cell DNA; WT PBMC=native PBMC DNA) for five different kidney-specific assays directed against differentially methylated regions (DMRs) of GRAMD1B, DDC, MAST4, MCF2L and PAX2. The amount of DNA amplified was calculated from a standard curve of bisulfite converted fully methylated DNA. Detection of differing amounts of kidney DNA is indicative of the cell-specificity of the different markers.

FIG. 9. A. Graph showing Ct values for 1 mL of ten plasma samples (<30 y.o.) amplified by qPCR for five different kidney-specific assays directed against differentially methylated regions of GRAMD1B, DDC, MAST4, MCF2L and PAX2 genes and also amplified with ACTB to show that DNA is present and to determine the total yield of amplifiable DNA from 1 mL plasma. B. The same data as in A., represented as a % of total cfDNA as determined relative to ACTB amplification.

FIG. 10. A. Graph showing concentration of cfDNA in copies per 1 ml of 20 presumed healthy urine samples (26-61 y.o.) amplified by dPCR for five different kidney-specific assays directed against differentially methylated regions of GRAMD1B, DDC, MAST4, MCF2L and PAX2 genes, and also amplified with ACTB to show that DNA is present and to determine the total yield of amplifiable DNA from 1 mL urine. B. The same data as in A., showing the mean amount of cfDNA/mL urine relative to age of donor. C. The same data as in B., represented as a % of total cfDNA as determined relative to ACTB amplification in age order of donors.

FIG. 11. Graphs showing total concentration of kidney specific cfDNA per mL plasma, amplified with kidney-specific assays directed against differentially methylated regions of GRAMD1B, DDC and PAX2 genes, and also amplified with ACTB, in samples taken pre- and post-renal transplant. Samples were collected from 25 individual patients immediately pre-transplant (0 hours), and/or at various times post-transplant (within 24 hours and/or 7 days (168 hours)) depending on the sample.

FIG. 12. Graphs showing methylation status of identified probes in different kidney cell types, namely bulk kidney tissue, renal cortical epithelial cells, renal proximal tubule epithelial cells and cultured podocytes.

FIG. 13, PCR Assay Development. A) Differentially methylated region of interest for PAX2. Whole Blood and tissue from sites other than kidney are hypomethylated whereas a kidney tissue is hypermethylated. Region where PCR amplicon was developed is indicated by the green box. B) The % DNA methylation determined using from the Illumina methylation array for the CpG closest to the PCR target region. C) The % methylation measured as per the digital PCR assay in 5 ng DNA from a range of tissue sources.

FIG. 14. Graphs showing PAX2, GRAMD1B and DDC methylation biomarker comparison in urine samples from subjects with various stages of CKD. ACTB used as a control.

FIG. 15. Graphs showing PAX2, GRAMD1B and DDC methylation biomarker comparison in urine samples from presumed healthy subjects with no known kidney disease and subjects pre- and post-heart transplant surgery.

FIG. 16. Graphs showing PAX2, GRAMD1B and DDC methylation biomarker comparison in plasma samples from presumed healthy subjects with no known kidney disease and subjects pre- and post-heart transplant surgery.

FIG. 17. Patient 1 serial testing. Graph A shows total cfDNA, Graphs B to D show PAX2, DDC and GRAMD1B methylation biomarker comparison in urine (left y-axis) and plasma (right y-axis) samples post heart transplant surgery. Graph E provides the standard of care markers for the patient, including creatinine (μmol/mL), eGFR (estimated glomerular filtration rate, ml/min/1.73 m²) and urine output (mL) using a rolling 6-hour average based on hourly readings.

FIG. 18. Patient 10 serial testing. Graph A shows total cfDNA, Graphs B to D show PAX2, DDC and GRAMD1B methylation biomarker comparison in urine (left y-axis) and plasma (right y-axis) samples post heart transplant surgery. Graph E provides the standard of care markers for the patient, including creatinine (μmol/mL), and eGFR (estimated glomerular filtration rate, ml/min/1.73 m²) and urine output (mL) using a rolling 6-hour average based on hourly readings.

FIG. 19. Graphs showing PAX2 methylation biomarker comparison in urine samples obtained from healthy cats and cats with CKD.

FIG. 20. Design of methylation specific PCR primers. The native strand of DNA for the differentially methylated region of interest is acquired (top strand) and in silico bisulphite converted either as fully methylated DNA whereby all cytosine (C) residues are converted to thymine (t) except when in the context of CG dinucleotide (middle strand), or fully unmethylated DNA whereby all cytosine (C) residues are converted to thymine (t) including when in the context of CG dinucleotide (bottom strand).

FIG. 21. DNA alignment between a region of the PAX2 gene for human, cat and dog sequences showing primers (underlined) and probes (bold) in regions of high sequence identity (identity is represented by a star under the aligned sequences).

FIG. 22. Analytical PCR results from testing a variety of human PAX2 assays designed within the region hg38; chr10: 100,745,582-100,829,944

FIG. 23. Analytical PCR results from testing a variety of human DDC assays designed within the region hg38; chr7: 50,458,443-50,565,405.

FIG. 24. Analytical PCR results from testing a variety of human GRAMD1B assays designed within the region hg38; chr11: 123,358,422-123,627,789.

FIG. 25. Analytical PCR results from testing a variety of human MAST4 assays designed within the region hg38; chr5: 66,596,393-67, 169,591.

FIG. 26. Analytical PCR results from testing a variety of human MCF2L assays designed within the region hg38; chr13: 112,969,214-113,099,742.

### DETAILED DESCRIPTION

### Definitions

In the context of this specification, the terms “a” and “an” are used herein to refer to one or to more than one (i.e. to at least one) of the grammatical object of the article. By way of example, “an element” means one element or more than one element.

The term “about” is understood to refer to a range of +/−10%, preferably +/−5% or +/−1% or, more preferably, +/−0.1%.

The terms “comprise”, “comprises”, “comprised” or “comprising”, “including” or “having” and the like in the present specification and claims are used in an inclusive sense, ie, to specify the presence of the stated features but not preclude the presence of additional or further features.

As used herein, a “CpG dinucleotide”, “CpG methylation site” or equivalent, shall be taken to denote a cytosine linked to a guanine by a phosphodiester bond. CpG dinucleotides are targets for methylation of the cytosine residue and may reside within coding or non-coding nucleic acids.

The term “identity” refers to a relationship between the sequences of two or more polypeptide molecules or two or more nucleic acid molecules, as determined by aligning and comparing the sequences. The percent identity between two sequences is a function of the number of identical positions shared by the sequences when the sequences are optimally aligned (ie, % homology=#of identical positions/total #of positions×100), with optimal alignment determined taking into account the number of gaps, and the length of each gap, which need to be introduced for optimal alignment of the two sequences. The comparison of sequences and determination of percent identity between two sequences can be accomplished using a mathematical algorithm.

The percent identity between two nucleotide sequences can be determined using the GAP program in the GCG software package, using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program, using a PAM120 weight residue table, a gap length penalty of 12 and a gap penalty of 4. In addition, the percent identity between two amino acid sequences can be determined using the Needleman and Wunsch (J. Mol. Biol. (48): 444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

As used herein, the term “DNA methylation” will be understood to mean the presence of a methyl group added by the action of a DNA methyl transferase enzyme to a cytosine base or bases in a region of nucleic acid e.g. genomic DNA. Accordingly, the term, “methylation status” as used herein refers to the presence or absence of methylation at a specific locus.

The term “substantially complementary” when used to describe a first nucleotide sequence in relation to a second nucleotide sequence, refers to the ability of an oligonucleotide or polynucleotide comprising the first nucleotide sequence to hybridize to, and form a duplex structure with, an oligonucleotide or polynucleotide comprising the second nucleotide sequence. It will be understood that the sequence of a nucleic acid need not be 100% complementary to that of its target. Conditions under which hybridisation occurs may be stringent, such as 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can also apply. Substantial complementarity allows the relevant function of the nucleic acid to proceed, eg, guide RNA hybridisation and CRISPR-mediated gene activation. The skilled person will be able to determine the set of conditions most appropriate for a test of complementarity of two sequences in accordance with the ultimate application of the hybridized nucleotides.

The term “subject” refers to an animal, preferably a mammal, such as a human or non-human including but not limited to members of the classifications of ovine, bovine, equine, porcine, feline, canine, primates and rodents, especially domesticated members of those classifications, such as, but not limited to, cats, sheep, cattle, horses, cats, dogs, pigs, chickens, rats and mice.

The term “reference level” in the context of the method of the invention refers to a level of a differentially methylated region in a subject that has no or insignificant organ damage, in particular no or insignificant kidney damage.

In one preferred embodiment, the present invention relates to methods described herein used in relation to human subjects. In particular, for example, the present invention relates to the use of methods described herein to detect kidney damage in humans. In an alternative embodiment, the present invention relates to, for example, methods described herein used in relation to non-human subjects. In particular, the use of methods described herein to detect kidney damage in, but not limited to, domesticated animals such as cats, sheep, cattle, horses, cats, dogs, pigs, chickens, rats and mice, including companion animals. In one particular embodiment, the present invention relates to, for example, methods described herein to detect kidney damage in cats and/or dogs.

The skilled person in the relevant art would understand that performing multiple alignments of nucleotide sequences is routine, using publicly available software such as, but not limited to, ClustalW. The skilled person will also readily understand that common primers and probes could be designed to detect regions of high nucleotide sequence identity in two or more different species. Alternatively, it would be clear to the skilled person that species-specific oligonucleotides could be designed to target DMRs from one species in particular. Differential methylation status can be determined in each target species for the method of the invention to be used as described in this application. Specifically, the differential methylation status can be determined using methodologies as detailed in this specification.

More specifically, the person of skill in the art will understand that primers and probes suitable for use in the methods described herein could be readily designed to detect DMRs in, for example, at least one locus selected from the group consisting of GRAMD1B, DDC, MAST4, MCF2L, PAX2, chr12-122277360 (CLIP1), chr17 35303285, DEF6, EMX1, HPD, PDE4D and SPAG5. In particular, the skilled person would understand relevant loci sequences, for example GRAMD1B, DDC, MAST4, MCF2L, PAX2, chr12-122277360 (CLIP1), chr17 35303285, DEF6, EMX1, HPD, PDE4D and SPAG5 from different subject species, such a humans and non-humans exhibit high levels of sequence identity. Thus, based on specific DMR sequences disclosed and exemplified herein, for example in relation to human and cat kidney-specific DMR sequences, the skilled person could readily identify kidney DMRs in a range of non-human animals such as, but not limited to, cats, dogs, sheep, cattle, horses, mice, rats, pigs and chickens. Relevant sequence information in relation to loci containing kidney-specific DMRs from human and non-human animals, such as domesticated animals, is set out in Tables 1 to 11 below.

Exemplary support for the identification of kidney-specific DMRs in non-human animals is provided in FIG. 19, which shows increased levels of PAX2 methylation biomarker in urine samples obtained from cats with CKD compared with urine obtained from healthy cats.

TABLE 1
Sequence information for PAX2 gene in humans and 9 representative non-human animals including domesticated animals

| | Gene | | | | RefSeq Transcript | |
|---|---|---|---|---|---|---|
| Gene ID | symbol | Description | Scientific name | Common name | accessions | Genomic location |
| 5076 | PAX2 | paired box 2 | Homo sapiens | human | NM_000278.5 | hg19; chr10: 102,505,468- |
| | | | | | | 102,590,402 |
| 101092147 | PAX2 | paired box 2 | Felis catus | domestic cat | XM_045040573.1 | felCat9; chrD2: 61,420,427- |
| | | | | | | 41,509,323 |
| 609228 | PAX2 | paired box 2 | Canis lupus familiaris | dog | XM_038440302.1 | canFam4; chr28: 13,623,521- |
| | | | | | | 13,709,770 |
| 18504 | Pax2 | paired box 2 | Mus musculus | house mouse | NM_001368747.1 | Mm39; chr19: 44,745,345- |
| | | | | | | 44,826,708 |
| 293992 | Pax2 | paired box 2 | Rattus norvegicus | Norway rat | NM_001106361.1 | Rn7; chr1: 243,616,745- |
| | | | | | | 243,695,321 |
| 100297382 | PAX2 | paired box 2 | Bos taurus | cattle | XM_015460715.2 | bosTau9; chr26: 21,589,730- |
| | | | | | | 21,679,960 |
| 100422802 | PAX2 | paired box 2 | Ovis aries | sheep | XM_027960132.2 | oviAri4; Chr22: 20,727,564- |
| | | | | | | 20,804,982 |
| 100070326 | PAX2 | paired box 2 | Equus caballus | horse | XM_023640845.1 | equCab2; chr1: 29,224,779- |
| | | | | | | 29,312,586 |
| 100515585 | PAX2 | paired box 2 | Sus scrofa | pig | XM_005671388.3 | susScr11; chr14: 111,822,334- |
| | | | | | | 111,914,950 |
| 395574 | PAX2 | paired box 2 | Gallus gallus | chicken | XM_040702254.2 | galGal6; chr6: 17,927,955- |
| | | | | | | 18,019,678 |

TABLE 2
Sequence information for GRAMD1B gene in humans and 9 representative non-human animals including domesticated animals

| | Gene | | | | RefSeq Transcript | |
|---|---|---|---|---|---|---|
| Gene ID | symbol | Description | Scientific name | Common name | accessions | Genomic location |
| 57476 | GRAMD1B | GRAM domain | Homo sapiens | human | NM_001367420 | Hg19; chr11: 123,229,130- |
| | | containing 1B | | | | 123,498,475 |
| 101092289 | GRAMD1B | GRAM domain | Felis catus | domestic cat | XM_023239234.2 | felCat9; chrD1: 20,404,459- |
| | | containing 1B | | | | 20,655 043 |
| 489852 | GRAMD1B | GRAM domain | Canis lupus | dog | XM_005619646.4 | canFam4; chr5: 10,840,820- |
| | | containing 1B | familiaris | | | 11,008,361 |
| 235283 | Gramd1b | GRAM domain | Mus musculus | house mouse | NM_172768.2 | Mm39; chr9: 40,204,529- |
| | | containing 1B | | | | 40,367,060 |
| 300644 | Gramd1b | GRAM domain | Rattus norvegicus | Norway rat | NM_001191616.1 | Rn7; chr8: 40,659,182- |
| | | containing 1B | | | | 40,821,791 |
| 517332 | GRAMD1B | GRAM domain | Bos taurus | cattle | XM_010812450.3 | bosTau9; chr15: 34,039,722- |
| | | containing 1B | | | | 34,226,170 |
| 101118986 | GRAMD1B | GRAM domain | Ovis aries | sheep | XM_027979340.2 | oviAri4; chr15: 33,537,469- |
| | | containing 1B | | | | 33,727,964 |
| 100063638 | GRAMD1B | GRAM domain | Equus caballus | horse | XM_023645036.1 | equCab3; chr7: 31,480,394- |
| | | containing 1B | | | | 31,643,572 |
| 100512979 | GRAMD1B | GRAM domain | Sus scrofa | pig | XM_021063027.1 | susScr11; chr9: 50,335,218- |
| | | containing 1B | | | | 50,507,328 |
| 769528 | GRAMD1B | GRAM domain | Gallus gallus | chicken | XM_025143357.1 | galGal6; chr24: 2,925,045- |
| | | containing 1B | | | | 2,954,570 |

TABLE 3
Sequence information for DDC gene in humans and 9 representative non-human animals including domesticated animals

| | Gene | | | | RefSeq Transcript | |
|---|---|---|---|---|---|---|
| Gene ID | symbol | Description | Scientific name | Common name | accessions | Genomic location |
| 1644 | DDC | dopa | Homo sapiens | human | NM_001082971.2 | hg19; chr7: 50,526,140- |
| | | decarboxylase | | | | 50,633,102 |
| 101082989 | DDC | dopa | Felis catus | domestic cat | XM_003982613.5 | felCat9; chrA2: 66,614,136- |
| | | decarboxylase | | | | 66,699,967 |
| 606852 | DDC | dopa | Canis lupus | dog | XM_022405156.2 | canFam4; chr18: 1,966,951- |
| | | decarboxylase | familiaris | | | 2,041,333 |
| 13195 | Ddc | dopa | Mus musculus | house mouse | NM_001190448.1 | Mm39; chr11: 11,764,101- |
| | | decarboxylase | | | | 11,848,144 |
| 24311 | Ddc | dopa | Rattus norvegicus | Norway rat | NM_012545.4 | Rn7; chr14: 86,378,685- |
| | | decarboxylase | | | | 86,469,175 |
| 280762 | DDC | dopa | Bos taurus | cattle | NM_173907.2 | bosTau9; chr4: 5,335,533- |
| | | decarboxylase | | | | 5,441,303 |
| 101106765 | DDC | dopa | Ovis aries | sheep | XM_012176274.4 | oviAri4; chr4: 5,274,312- |
| | | decarboxylase | | | | 5,377,534 |
| 100052557 | DDC | dopa | Equus caballus | horse | XM_001498321.4 | equCab3; chr4: 20,269,062- |
| | | decarboxylase | | | | 20,354,098 |
| 396857 | DDC | dopa | Sus scrofa | pig | NM_213854.2 | susScr11; chr9: 136,496,366- |
| | | decarboxylase | | | | 136,548,046 |
| 420947 | DDC | dopa | Gallus gallus | chicken | XM_419032.6 | galGal6; chr2: 80,753,570- |
| | | decarboxylase | | | | 80,827,081 |

TABLE 4
Sequence information for MAST4 gene in humans and 9 representative non-human animals including domesticated animals

| | Gene | | | | RefSeq Transcript | |
|---|---|---|---|---|---|---|
| Gene ID | symbol | Description | Scientific name | Common name | accessions | Genomic location |
| 375449 | MAST4 | microtubule associated | Homo | human | NM_001164664.2 | Hg19; chr5: 65,892,221- |
| | | serine/threonine kinase | sapiens | | | 66,465,421 |
| | | family member 4 | | | | |
| 101087553 | MAST4 | microtubule associated | Felis catus | domestic cat | XM_023257117.2 | felCat9; chrA1: 135,824,381- |
| | | serine/threonine kinase | | | | 136,392,000 |
| | | family member 4 | | | | |
| 612904 | MAST4 | microtubule associated | Canis lupus | dog | XM_038454835.1 | canFam4; chr2: 51,332,587- |
| | | serine/threonine kinase | familiaris | | | 51,871,159 |
| | | family member 4 | | | | |
| 328329 | Mast4 | microtubule associated | Mus | house mouse | NM_175171.3 | Mm39; chr13: 102,868,999- |
| | | serine/threonine kinase | musculus | | | 103,470,968 |
| | | family member 4 | | | | |
| 103690045 | Mast4 | microtubule associated | Rattus | Norway rat | XM_039103957.1 | Rn7; chr2: 33,893,266- |
| | | serine/threonine kinase | norvegicus | | | 34,061,192 |
| | | family member 4 | | | | |
| 529061 | MAST4 | microtubule associated | Bos taurus | cattle | XM_024981444.1 | bosTau9; chr20: 12,442,479- |
| | | serine/threonine kinase | | | | 13,060,905 |
| | | family member 4 | | | | |
| 101122306 | MAST4 | microtubule associated | Ovis aries | sheep | XM_027980036.2 | oviAri4; chr16: 12,423,548- |
| | | serine/threonine kinase | | | | 13,045,109 |
| | | family member 4 | | | | |
| 100056646 | MAST4 | microtubule associated | Equus | horse | XM_023625593.1 | equCab3; chr21: 7,129,811- |
| | | serine/threonine kinase | caballus | | | 7,707,227 |
| | | family member 4 | | | | |
| 100514689 | MAST4 | microtubule associated | Sus scrofa | pig | XM_021076748.1 | susScr11; chr16: 44,972,519- |
| | | serine/threonine kinase | | | | 45,562,100 |
| | | family member 4 | | | | |
| 427169 | MAST4 | microtubule associated | Gallus gallus | chicken | XM_015277620.2 | galGal6; chrZ: 21,008,240- |
| | | serine/threonine kinase | | | | 21,296,595 |
| | | family member 4 | | | | |

TABLE 5
Sequence information for MCF2L gene in humans and 9 representative non-human animals including domesticated animals

| | Gene | | | | RefSeq Transcript | |
|---|---|---|---|---|---|---|
| Gene ID | symbol | Description | Scientific name | Common name | accessions | Genomic location |
| 23263 | MCF2L | MCF.2 cell line derived | Homo | human | NM_001112732.3 | hg19; chr13: 113,623,528- |
| | | transforming sequence like | sapiens | | | 113,754,056 |
| 101090322 | MCF2L | MCF.2 cell line derived | Felis catus | domestic | XM_019829789.1 | felCat9; chrA1: 82,102,195- |
| | | transforming sequence like | | cat | | 82,234,328 |
| 485555 | MCF2L | MCF.2 cell line derived | Canis lupus | dog | XM_014106461.3 | canFam4; chr22: 60,598,080- |
| | | transforming sequence like | familiaris | | | 60,727,816 |
| 17207 | Mcf2l | mcf.2 transforming sequence- | Mus | house | NM_178076.4 | Mm39; chr8: 12,965,911- |
| | | like | musculus | mouse | | 13,070,502 |
| 117020 | Mcf2l | MCF.2 cell line derived | Rattus | Norway | NM_001413769.1 | Rn7; chr16: 76,507,900- |
| | | transforming sequence-like | norvegicus | rat | | 76,612,582 |
| 505595 | MCF2L | MCF.2 cell line derived | Bos taurus | cattle | XM_025000178.1 | bosTau9; chr12: 86,399,246- |
| | | transforming sequence like | | | | 86,489,884 |
| 101118115 | MCF2L | MCF.2 cell line derived | Ovis aries | sheep | XM_027973813.2 | oviAri4; chr1: 201,711,850- |
| | | transforming sequence like | | | | 201,952,818 |
| 100067048 | MCF2L | MCF.2 cell line derived | Equus | horse | XM_023621866.1 | equCab3; chr17: 79,632,962- |
| | | transforming sequence like | caballus | | | 79,811,986 |
| 100518447 | MCF2L | MCF.2 cell line derived | Sus scrofa | pig | XM_021065947.1 | susScr11; chr11: 78,407,525- |
| | | transforming sequence like | | | | 78,505,495 |
| 418748 | MCF2L | MCF.2 cell line derived | Gallus gallus | chicken | NM_001252063.1 | galGal6; chr1: 138,951,176- |
| | | transforming sequence like | | | | 139,094,938 |

TABLE 6
Sequence information for DEF6 gene in humans and 9 representative non-human animals including domesticated animals

| | Gene | | | | RefSeq Transcript | |
|---|---|---|---|---|---|---|
| Gene ID | symbol | Description | Scientific name | Common name | accessions | Genomic location |
| 50619 | DEF6 | DEF6 guanine nucleotide | Homo | human | NM_022047.4 | Hg319; chr6: 35,265,595- |
| | | exchange factor | sapiens | | | 35,289,548 |
| 101098142 | DEF6 | DEF6 guanine nucleotide | Felis catus | domestic | XM_023253893.2 | felCat9; chrB2: 36,686,920- |
| | | exchange factor | | cat | | 36,705,932 |
| 608163 | DEF6 | DEF6 guanine nucleotide | Canis lupus | dog | XM_014118384.3 | canFam4; chr12: 4.640.733- |
| | | exchange factor | familiaris | | | 4.661.670 |
| 23853 | Def6 | differentially expressed in | Mus | house | NM_027185.3 | Mm39; chr17: 28,426,752- |
| | | FDCP 6 | musculus | mouse | | 28,447,582 |
| 309642 | Def6 | DEF6 guanine nucleotide | Rattus | Norway rat | NM_001191717.2 | Rn7; chr20: 6,268,614- |
| | | exchange factor | norvegicus | | | 6,289,507 |
| 516457 | DEF6 | DEF6 guanine nucleotide | Bos taurus | cattle | NM_001098994.1 | bosTau9; chr23: 9,271,537- |
| | | exchange factor | | | | 9,298,623 |
| 101117371 | DEF6 | DEF6 guanine nucleotide | Ovis aries | sheep | XM_004018767.5 | oviAri4; chr20: 9,333,195- |
| | | exchange factor | | | | 9,356,811 |
| 100053351 | DEF6 | DEF6 guanine nucleotide | Equus | horse | XM_005603837.3 | equCab3; chr20: 36,312,215- |
| | | exchange factor | caballus | | | 36,331,906 |
| 100154998 | DEF6 | DEF6 guanine nucleotide | Sus scrofa | pig | XM_001929239.6 | susScr11; chr7: 31,191,788- |
| | | exchange factor | | | | 31,216,436 |
| 419893 | DEF6 | DEF6 guanine nucleotide | Gallus gallus | chicken | XM_015299006.2 | galGal6; chr26: 4,108,437- |
| | | exchange factor | | | | 4,119,833 |

TABLE 7
Sequence information for EMX1 gene in humans and 9 representative
non-human animals including domesticated animals

| | Gene | | | | RefSeq Transcript | |
|---|---|---|---|---|---|---|
| Gene ID | symbol | Description | Scientific name | Common name | accessions | Genomic location |
| 2016 | EMX1 | empty spiracles | Homo sapiens | human | NM_004097.3 | Hg19; chr2: 73,144,648- |
| | | homeobox 1 | | | | 73,162,020 |
| 101097140 | EMX1 | empty spiracles | Felis catus | domestic | XM_023251768.2 | felCat9; chrA3: 91,890,003- |
| | | homeobox 1 | | cat | | 91,912,913 |
| 119869779 | EMX1 | empty spiracles | Canis lupus | dog | XM_038453437.1 | canFam4; chr17: 50,410,143- |
| | | homeobox 1 | familiaris | | | 50,428,602 |
| 13796 | Emx1 | empty spiracles | Mus musculus | house | NM_010131.2 | Mm39; chr6: 85,164,420- |
| | | homeobox 1 | | mouse | | 85,181,340 |
| 500235 | Emx1 | empty spiracles | Rattus | Norway rat | XM_575584.6 | Rn7; chr4: 117,725,148- |
| | | homeobox 1 | norvegicus | | | 117,741,616 |
| 539333 | EMX1 | empty spiracles | Bos taurus | cattle | NM_001192223.1 | bosTau9; chr11: 11,505,904- |
| | | homeobox 1 | | | | 11,523,111 |
| 101103146 | EMX1 | empty spiracles | Ovis aries | sheep | XM_027966995.2 | oviAri4; chr3: 94,717,557- |
| | | homeobox 1 | | | | 94,735,130 |
| 100059311 | EMX1 | empty spiracles | Equus caballus | horse | XM_023618899.1 | equCab3; chr15: 30,977,952- |
| | | homeobox 1 | | | | 30,995,172 |
| 100521187 | EMX1 | empty spiracles | Sus scrofa | pig | XM_021087381.1 | susScr11; chr3: 69,842,061- |
| | | homeobox 1 | | | | 69,863,940 |
| 768977 | EMX1 | empty spiracles | Gallus gallus | chicken | XM_001232150.5 | galGal6; chr4: 89,847,691- |
| | | homeobox 1 | | | | 89,856,291 |

TABLE 8
Sequence information for HPD gene in humans and 9 representative non-human animals including domesticated animals

| | Gene | | | | RefSeq Transcript | |
|---|---|---|---|---|---|---|
| Gene ID | symbol | Description | Scientific name | Common name | accessions | Genomic location |
| 3242 | HPD | 4-hydroxyphenylpyruvate | Homo sapiens | human | NM_002150.3 | Hg19; chr12: 122,277,433- |
| | | dioxygenase | | | | 122,296,765 |
| 101093465 | HPD | 4-hydroxyphenylpyruvate | Felis catus | domestic cat | XM_003994634.4 | felCat9; chrD3: 7,765,791- |
| | | dioxygenase | | | | 7,776,592 |
| 610778 | HPD | 4-hydroxyphenylpyruvate | Canis lupus | dog | XM_848329.6 | canFam4; chr15: 15,111,689- |
| | | dioxygenase | familiaris | | | 15,121,352 |
| 15445 | Hpd | 4-hydroxyphenylpyruvic | Mus musculus | house mouse | NM_008277.3 | Mm39; chr5: 123,309,870- |
| | | acid dioxygenase | | | | 123,320,790 |
| 29531 | Hpd | 4-hydroxyphenylpyruvate | Rattus | Norway rat | NM_017233.2 | Rn7; chr12: 33,381,397- |
| | | dioxygenase | norvegicus | | | 33,392,750 |
| 516058 | HPD | 4-hydroxyphenylpyruvate | Bos taurus | cattle | NM_001015611.1 | bosTau9; chr17: 53,458,851- |
| | | dioxygenase | | | | 53,470,907 |
| 101122814 | HPD | 4-hydroxyphenylpyruvate | Ovis aries | sheep | XM_004017344.4 | oviAri4; chr17: 53,016,926- |
| | | dioxygenase | | | | 53,028,870 |
| 100059853 | HPD | 4-hydroxyphenylpyruvate | Equus caballus | horse | XM_005612614.3 | equCab3; chr8: 24,681,064- |
| | | dioxygenase | | | | 24,690,704 |
| 397443 | HPD | 4-hydroxyphenylpyruvate | Sus scrofa | pig | NM_001348964.1 | susScr11; chr14: 30,845,613- |
| | | dioxygenase | | | | 30,857,658 |
| 416852 | HPD | 4-hydroxyphenylpyruvate | Gallus gallus | chicken | NM_001277491.1 | galGal6; chr15: 5,984,672- |
| | | dioxygenase | | | | 5,988,411 |

TABLE 9
Sequence information for PDE4D gene in humans and 9 representative non-human animals including domesticated animals

| | Gene | | | | RefSeq Transcript | |
|---|---|---|---|---|---|---|
| Gene ID | symbol | Description | Scientific name | Common name | accessions | Genomic location |
| 5144 | PDE4D | phosphodiesterase 4D | Homo sapiens | human | NM_001104631.2 | Hg19; chr5: 58,264,865- |
| | | | | | | 59,064,674 |
| 101092826 | PDE4D | phosphodiesterase 4D | Felis catus | domestic cat | XM_023256805.2 | felCat9; chrA1: 129,266,245- |
| | | | | | | 130,126,217 |
| 487221 | PDE4D | phosphodiesterase 4D | Canis lupus | dog | XM_022415225.1 | canFam4; chr2: 45,031,308- |
| | | | familiaris | | | 42,313,972 |
| 238871 | Pde4d | phosphodiesterase 4D, | Mus musculus | house mouse | NM_001402884.1 | Mm39; chr13: 109,396,854- |
| | | cAMP specific | | | | 110,092,498 |
| 24627 | Pde4d | phosphodiesterase 4D | Rattus | Norway rat | NM_001113328.2 | Rn7; chr2: 40,723,169- |
| | | | norvegicus | | | 41,524,760 |
| 539556 | PDE4D | phosphodiesterase 4D | Bos taurus | cattle | XM_003587511.4 | bosTau9; chr20: 18,742,880- |
| | | | | | | 20,317,769 |
| 101108953 | PDE4D | phosphodiesterase 4D | Ovis aries | sheep | XM_027980061.2 | oviAri4; chr16: 18,798,128- |
| | | | | | | 20,387,125 |
| 100051344 | PDE4D | phosphodiesterase 4D | Equus caballus | horse | XM_023625244.1 | equCab3; chr21: 13,159,332- |
| | | | | | | 14,489,780 |
| 100037967 | PDE4D | phosphodiesterase 4D | Sus scrofa | pig | XM_021076863.1 | susScr11; chr16: 37,897,300- |
| | | | | | | 38,353,825 |
| 769469 | PDE4D | phosphodiesterase 4D | Gallus gallus | chicken | XM_004937253.2 | galGal6; chrZ: 18,249,788- |
| | | | | | | 18,686,970 |

TABLE 10
Sequence information for SPAG5 gene in humans and 9 representative non-human animals including domesticated animals

| | Gene | | | | RefSeq Transcript | |
|---|---|---|---|---|---|---|
| Gene ID | symbol | Description | Scientific name | Common name | accessions | Genomic location |
| 10615 | SPAG5 | sperm associated | Homo sapiens | human | NM_006461.4 | Hg19; chr17: 26,904,592- |
| | | antigen 5 | | | | 26,926,043 |
| 101098415 | SPAG5 | sperm associated | Felis catus | domestic cat | XM_003996487.5 | felCat9; chrE1: 18,199,182- |
| | | antigen 5 | | | | 18,219,439 |
| 480623 | SPAG5 | sperm associated | Canis lupus | dog | XM_038677464.1 | canFam4; chr9: 42,755,160- |
| | | antigen 5 | familiaris | | | 42,776,506 |
| 54141 | Spag5 | sperm associated | Mus musculus | house mouse | NM_017407.3 | Mm39; chr11: 78,192,355- |
| | | antigen 5 | | | | 78,213,283 |
| 252918 | Spag5 | sperm associated | Rattus | Norway rat | NM_001044224.1 | Rn7; chr10: 63,198,768- |
| | | antigen 5 | norvegicus | | | 63,216,746 |
| 504585 | SPAG5 | sperm associated | Bos taurus | cattle | NM_001205549.1 | bosTau9; chr19: 20,034,503- |
| | | antigen 5 | | | | 20,053,527 |
| 101105670 | SPAG5 | sperm associated | Ovis aries | sheep | XM_027975234.2 | oviAri4; chr7: 33,000,994- |
| | | antigen 5 | | | | 33,101,122 |
| 100059272 | SPAG5 | sperm associated | Equus caballus | horse | XM_001918338.3 | equCab3; chr11: 42,848,974- |
| | | antigen 5 | | | | 42,869,113 |
| 100286810 | SPAG5 | sperm associated | Sus scrofa | pig | NM_001159311.1 | susScr11; chr12: 44,827,947- |
| | | antigen 5 | | | | 44,848,527 |
| 771702 | SPAG5 | sperm associated | Gallus gallus | chicken | XM_025142076.1 | galGal6; chr19: 5,889,763- |
| | | antigen 5 | | | | 5,897,953 |

TABLE 11
Sequence information for CLIP1 gene in humans and 9 representative non-human animals including domesticated animals

| | Gene | | | | RefSeq Transcript | |
|---|---|---|---|---|---|---|
| Gene ID | symbol | Description | Scientific name | Common name | accessions | Genomic location |
| 6249 | CLIP1 | CAP-Gly domain | Homo sapiens | human | NM_001247997.2 | Hg19; chr12: 122,756,016- |
| | | containing linker protein 1 | | | | 122,907,216 |
| 101091733 | CLIP1 | CAP-Gly domain | Felis catus | domestic cat | XM_023241360.2 | felCat9; chrD3: 7,309,405- |
| | | containing linker protein 1 | | | | 7,435,193 |
| 477461 | CLIP1 | CAP-Gly domain | Canis lupus | dog | XM_038574931.1 | canFam4; chr26: 7,193,979- |
| | | containing linker protein 1 | familiaris | | | 7,318,170 |
| 56430 | Clip1 | CAP-GLY domain | Mus musculus | house mouse | NM_019765.5 | Mm39; chr5: 123,715,858- |
| | | containing linker protein 1 | | | | 123,822,681 |
| 65201 | Clip1 | CAP-GLY domain | Rattus | Norway rat | NM_001408966.1 | Rn7; chr12: 32,910,932- |
| | | containing linker protein 1 | norvegicus | | | 33,017,891 |
| 534624 | CLIP1 | CAP-Gly domain | Bos taurus | cattle | XM_024977349.1 | bosTau9; chr17: 52,998,859- |
| | | containing linker protein 1 | | | | 53,115,714 |
| 101121120 | CLIP1 | CAP-Gly domain | Ovis aries | sheep | XM_015101612.3 | oviAri4; chr17: 52,560,405- |
| | | containing linker protein 1 | | | | 52,672,565 |
| 100058794 | CLIP1 | CAP-Gly domain | Equus caballus | horse | XM_023647220.1 | equCab3; chr8: 25,002,515- |
| | | containing linker protein 1 | | | | 25,101,823 |
| 100153677 | CLIP1 | CAP-Gly domain | Sus scrofa | pig | XM_021072958.1 | susScr11; chr14: 30,320,870- |
| | | containing linker protein 1 | | | | 30,467,095 |
| 395784 | CLIP1 | CAP-Gly domain | Gallus gallus | chicken | XM_015275182.2 | galGal6; chr15: 6,112,206- |
| | | containing linker protein 1 | | | | 6,174,094 |

Where numerical ranges are used to describe certain embodiments of the present disclosure, it will be understood that each range should be considered to encompass subranges therein. For example, the description of a range such as from 1 to 6 should be considered to include subranges such as from 1 to 5, from 2 to 4, from 2 to 6 and so on. Likewise, the description of a range of between 1 and 6 should be considered to include subranges such as between 2 and 5, between 1 and 3, between 3 and 6 and so on.

### Cell Damage and Associated Conditions

Organ and tissue damage leading to organ- or tissue-specific cell death results in an increased concentration of organ- or tissue-specific DNA in the cfDNA population. The present inventors have found epigenetic signatures within different organs, tissues and even cell types that can be used as markers to detect organ, tissue or cell damage from a sample of cfDNA. For example, an increased concentration of kidney-specific epigenetic markers within cfDNA may be indicative of kidney damage resulting from cell death within the kidney. The methods described herein may be used to diagnose a disease or condition or disorder associated with cell death. The methods may also be used to prognose the likelihood of an event occurring. For example, the methods described herein may be used to prognose kidney failure in a subject.

The methods described herein are not limited to any particular organ or tissue. For example, the methods may be used to detect damage of the kidney, liver, spleen, prostate, heart, muscle, lungs, brain, small intestine, large intestine, bladder, pancreas, adrenal glands, breast, colon, pancreas, bone, placenta or skin, or a tissue or cell-type thereof. The damage may be caused by disease, infection or trauma.

In some examples, the methods described herein detect epigenetic markers in cfDNA from dead neurons (indicative of traumatic brain injury, amyotrophic lateral sclerosis, stroke, Alzheimer's disease, Parkinson's disease or brain tumors), dead pancreatic acinar cells (indicative of pancreatic cancer or pancreatitis), dead lung cells (indicative of lung pathologies including lung cancer), dead adipocytes (indicative of altered fat turnover), dead hepatocytes (indicative of liver failure, liver disease or liver toxicity), dead cardiomyocytes (indicative of cardiac disease or graft failure in the case of cardiac transplantation), dead skeletal muscle cells (indicative of muscle injury and myopathies), dead oligodendrocytes (indicative of relapsing multiple sclerosis, white matter damage in amyotrophic lateral sclerosis or glioblastoma), dead placental cells (indicative of pre-eclampsia or placental abruption) or dead colon cells (indicative of colorectal cancer).

In some examples, the methods described herein are used to detect kidney damage. Damage to the kidney may arise, for example, from acute kidney injury (AKI) or chronic kidney disease (CKD). At-risk groups for whom regular screening may be particularly beneficial include diabetics, subjects with high blood pressure, subjects with polycystic kidney disease, transplant recipients and so on.

Kidney disease is a major health problem that represents an interlinked spectrum of AKI and CKD. AKI is associated with high morbidity, mortality, prolonged hospital stay and progression to CKD. Patients with CKD can progress to End Stage Renal Disease (ESRD) where they require dialysis or a kidney transplant. Kidney disease has a large, measurable cost to the health system and is disproportionately prevalent in Indigenous Australians, lower socio-economic groups, the elderly and people in rural/remote locations. Kidney disease is under-diagnosed and most people are unaware of its presence until symptoms manifest. In the US, about 200,000 people (0.06% of US population) are living with a kidney transplant, about 4,000,000 (1.2%) will have an AKI every year and about 37,000,000 (11.2%) are estimated to be living with CKD. With 90% unaware of their diagnosis, CKD patients are 20 times more likely to die from cardiovascular disease, due to having CKD, than receive renal replacement. Early and accurate detection of kidney disease slows and reduces loss of kidney function. The use of serum creatinine as a surrogate to detect AKI/CKD takes up to 48 hours to yield measurable changes. For CKD, up to 50% of the kidney can be degraded prior to changes in creatinine and up to 90% before there are symptoms. The ability to detect these conditions earlier, in populations at greater risk and in patients with no overt clinical symptoms, will enable quicker intervention to reduce further kidney damage and reduce the morbidity and mortality associated with AKI/CKD. The present disclosure provides methods that are useful in the early detection of AKI and CKD.

The methods described herein may also be used to detect tissue or organ damage following a tissue or organ transplant. For example, the methods may be used to detect kidney damage following a kidney transplant. The methods may identify early signs of organ rejection. The methods may also be used to detect tissue or organ damage following a particular treatment. For example, the methods may be used to detect kidney damage following cardiothoracic surgery or renal replacement therapy.

The methods described herein do not rely upon detecting unique DNA sequences, and as such, they are not limited to settings in which the genome of one subject is to be distinguished from the genome of another subject (eg, following an organ or tissue transplant). In other words, the presently disclosed methods can be used to detect damage of a subject's own organ. In that regard, in the methods of the present disclosure, the subject and the organ are preferably autologous.

The epigenetic markers described herein may be organ-specific, tissue-specific or cell-specific, and in that regard, the methods of the present disclosure may be used to detect organ, tissue or cell damage. In the kidney, for example, the present inventors have identified epigenetic markers (eg, methylation in MAST4 and DDC) which are enriched in renal proximal tubule epithelial cells relative to other cells of the kidney and other organs of the body. Those markers may be used to diagnose conditions associated with or caused by damage to renal proximal tubule epithelial cells, such as ischemic reperfusion injury. Renal Proximal Tubule Epithelial Cells (RPTECs) have a large abundance of mitochondria with a heavy dependence on oxidative phosphorylation, making them vulnerable to injury and early markers for ischemic reperfusion injury through cell death. Injury to the RPTECs can also result in the formation of atubular glomeruli, leading to CKD. The ability to detect RPTEC specific cell death will improve early detection and location specific injury relevant to injury from surgery and disease.

In some examples, the present disclosure provides a method of detecting renal proximal tubule epithelial cell damage in a subject, the method comprising detecting methylation status at MAST4 or DDC in cfDNA obtained from a biological sample of the subject, wherein presence of methylated MAST4 or DDC in cfDNA is indicative of renal proximal tubule epithelial cell damage. It will be understood that increased levels of methylated MAST4 and/or DDC DNA in cfDNA obtained from a subject may be indicative that the subject is suffering from renal proximal tubule epithelial cell damage. In some examples, the present disclosure provides a method of diagnosing ischemic reperfusion injury in a subject, the method comprising detecting methylation status at MAST4 or DDC in cfDNA obtained from a biological sample of the subject.

In one embodiment, the method of the invention uses a combination of kidney differentially methylated regions to obtain a measurement of kidney injury. In this regard, the present invention provides kidney differentially methylated regions that correlate with particular kidney cells. For example, PAX2 in urine serves as a useful marker for general kidney damage. However, Pax2 is not an optimal classifier for stage two samples in CKD. In contrast, GRAMD1B and DDC, which correlate to kidney podocytes and renal proximal tubule cells, offer more effective options for detecting kidney damage issues. Moreover, PAX2 is likely to be susceptible to variability resulting from inflammation caused by, for example, infections. In the case of kidney transplant, it is known that the BK virus can cause an increase in kidney cfDNA in urine when measuring dd-cfDNA, which is expected as the infection is kidney-based. Consequently, the measurement of tubular markers is likely to provide a more robust assessment as they are less likely to be affected by the infection. Thus, measuring PAX2 enables a general determination of an issue in the kidney, other kidney DMRs provide a more specific indication of kidney cell damage. For example, detection of elevated levels of GRAMD1B indicates kidney podocytes damage and detection of elevated levels of DDC indicates renal proximal tubule cell damage.

The methods described herein may also be useful in detecting proliferative diseases such as cancer. Such diseases may be associated not only with increased cell proliferation but also with increased cell damage or cell death. For example, the proliferating cells themselves (eg, tumor cells) may die over time, releasing DNA that can be detected in cfDNA using the methods described herein. In other examples, the proliferating cells may cause damage or death to proximal or distal cells which thus release DNA that can be detected in cfDNA using the methods described herein.

The present disclosure also contemplates treatment of subjects found to be suffering from, or at risk of, organ or tissue damage. Treatment may include, for example, administration of a medicament, surgery, chemotherapy, lifestyle change, dietary change or physical therapy. In some examples, the present disclosure provides a method of detecting organ damage in a subject, the method comprising: detecting an organ-specific epigenetic marker in cfDNA obtained from a biological sample of the subject, wherein the presence of the epigenetic marker in the cfDNA is indicative of organ damage; and, if organ damage is detected, administering to the subject a treatment for the organ damage. In some examples, the present disclosure provides a method of treating a subject suffering from organ damage or at risk of suffering from organ damage, wherein the organ damage has been detected by a method comprising detecting an organ-specific epigenetic marker in cfDNA obtained from a biological sample of the subject, wherein the presence of the epigenetic marker in the cfDNA is indicative of organ damage.

### Epigenetic Markers

The epigenetic markers of the present disclosure are cell-specific, tissue-specific or organ-specific in the sense that they are enriched in that cell, tissue or organ relative to other cells, tissues or organs of the body. In some examples, the epigenetic marker is at least about 5% more abundant, such as at least about 10% more abundant, or at least about 15% more abundant, at least about 20% more abundant, at least about 25% more abundant, at least about 30% more abundant, at least about 35% more abundant, at least about 40% more abundant, at least about 45% more abundant, at least about 50% more abundant, at least about 55% more abundant, at least about 60% more abundant, at least about 65% more abundant, at least about 70% more abundant, at least about 75% more abundant, at least about 80% more abundant, at least about 85% more abundant, at least about 90% more abundant, at least about 95% more abundant or at least about 100% more abundant in the cell, tissue or organ of interest relative to other cells, tissues or organs of the body. In some examples, the epigenetic marker is at least 2-fold more abundant, at least 3-fold more abundant, at least 4-fold more abundant, at least 5-fold more abundant, at least 6-fold more abundant, at least 7-fold more abundant, at least 8-fold more abundant, at least 9-fold more abundant, at least 10-fold more abundant, at least 11-fold more abundant, at least 12-fold more abundant, at least 13-fold more abundant, at least 14-fold more abundant or at least 15-fold more abundant in the cell, tissue or organ of interest relative to other cells, tissues or organs of the body. In some examples, the epigenetic marker is only detectable in the cell, tissue or organ of interest.

Those skilled in the art will understand that different types of epigenetic markers may be used to identify specific cell-, tissue- or organ-types. Suitable epigenetic markers may include acetylation status of DNA or histones, or methylation status of DNA or histones. In some examples, the epigenetic marker is selected from the group consisting of a DNA modification, a histone modification and nucleosome positioning.

In some examples, nucleosome positioning is determined by a nucleosome positioning assay. Histone modifications may be detected by a pull-down assay using antibodies specific for a histone modification. The antibodies may be specific for histone methylation, acetylation, phosphorylation, ubiquitylation, GlcNAcylation, citrullination, krotonilation, or isomerization. In some examples, the histone methylation-specific antibodies comprise antibodies against H3K4Me1, H3K4Me2, H3K4Me3, or H3K36Me3 modifications.

Preferably, the epigenetic marker used in the methods of the present disclosure is DNA methylation status at one or more DMRs within the cfDNA. For example, a DMR may be unmethylated in the heart but methylated in other parts of the body. In some examples, the DMR is methylated in the kidney but unmethylated in other parts of the body. The amount of methylated DNA at the DMR that is present in cfDNA of a subject may be proportional to the level of organ damage experienced by the subject.

Methylation status may be detected at one or more CpG dinucleotides. For example, one epigenetic marker may have methylation at one CpG dinucleotide, two CpG dinucleotides, three CpG dinucleotides, four CpG dinucleotides, five CpG dinucleotides, six CpG dinucleotides, seven CpG dinucleotides, eight CpG dinucleotides, nine CpG dinucleotides, 10 CpG dinucleotides, 11 CpG dinucleotides, 12 CpG dinucleotides, 13 CpG dinucleotides, 14 CpG dinucleotides, 15 CpG dinucleotides, 16 CpG dinucleotides, 17 CpG dinucleotides, 18 CpG dinucleotides, 19 CpG dinucleotides, or at least 20 CpG dinucleotides. In circumstances where a DMR comprises more than one differentially methylated CpG dinucleotide, those CpG dinucleotides may be consecutive (ie, contiguous) or not consecutive within the DMR.

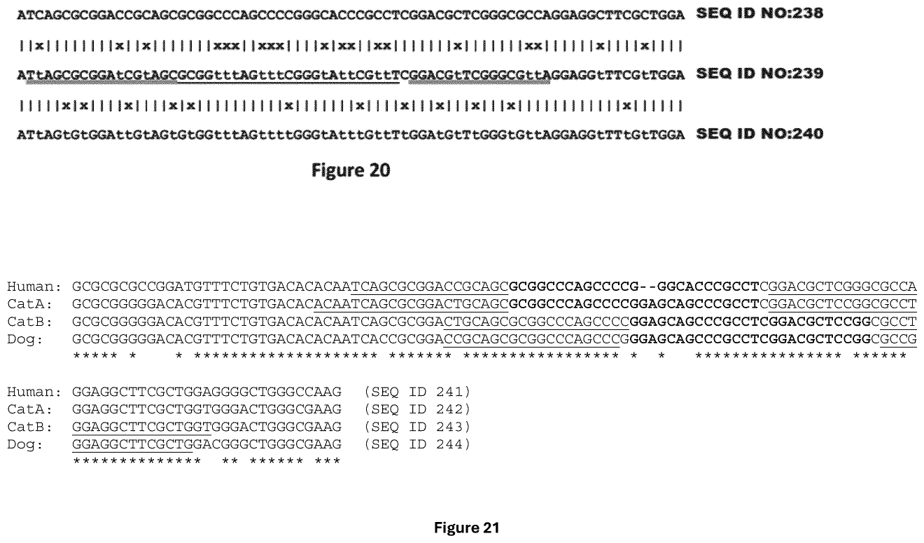

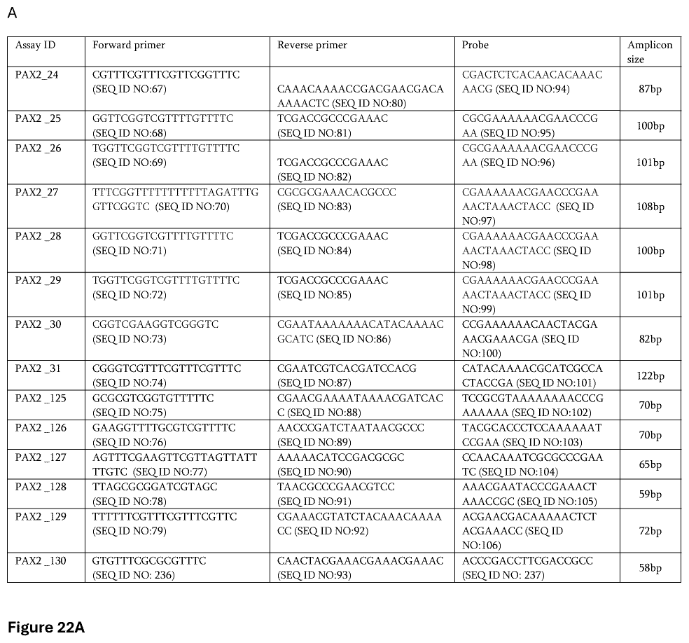

Various techniques may be used to detect DNA methylation status. For example, the cfDNA may be treated with bisulfite and sequenced or assayed using a PCR technique. Bisulfite conversion typically involves treating DNA with a bisulfite such as sodium bisulfite, leading to deamination of unmethylated cytosines into uracils, while methylated cytosines (both 5-methylcytosine and 5-hydroxymethylcytosine) remain unchanged. This is illustrated in FIG. 20. The DNA can then be amplified by PCR where the uracils are converted to thymines. Bisulfite converted DNA can be analysed for methylation status using primers that differentiate between methylated and unmethylated sequences. Primers may be designed such that amplification only occurs (or is substantially more efficient) when the template is derived from either methylated or unmethylated DNA. In addition, or alternatively, probes may be designed that specifically hybridise to bisulfite-converted DNA that is derived from either methylated or unmethylated DNA. It will be understood that following bisulfite-conversion of DNA, the two strands are often no longer complementary to each other, meaning that primers and probes to either strand may be designed. In some examples, the methylation status is detected using quantitative PCR (qPCR), digital PCR (dPCR) or digital droplet PCR (ddPCR). These techniques are described in Shemer, R. et al., Current Protocols in Molecular Biology, 127.1 (2019): e90; and Zemmour, Hai, et al., Nature Communications, 9.1 (2018): 1-9. Unmethylated cytosine nucleotides can also be enzymatically converted to uracil nucleotides, for example, using NEBNext's Enzymatic Methyl-seq Kit. The Methyl-seq Kit uses TET2 to oxidise 5-methylcytosine and 5-hydroxymethylcytosine, thereby protecting those methylated cytosines from deamination by apolipoprotein B mRNA-editing enzyme, catalytic polypeptide (APOBEC). In some examples, the methylation status may be detected using a nanopore sequencing technology. The nanopore sequencing technology may detect native CpG methylation in cfDNA without prior bisulfite treatment.

Alternatively, the amplification product of bisulfite-converted DNA may be sequenced to determine the methylation status of the template-comparing the sequence of the converted DNA to untreated DNA creates a methylation profile of the amplified region. The presence of a mutated or non-mutated nucleotide in a bisulfite-treated sample may also be detected using pyrosequencing, such as, for example, as described in Uhlmann et al., Electrophoresis, 23:4072-4079, 2002. Essentially this method is a form of real-time sequencing that uses a primer that hybridizes to a site adjacent or close to the site of a cytosine that is methylated. Following hybridization of the primer and template in the presence of a DNA polymerase each of four modified deoxynucleotide triphosphates are added separately according to a predetermined dispensation order. Only an added nucleotide that is complementary to the bisulfite treated sample is incorporated and inorganic pyrophosphate (PPi) is liberated. The PPi then drives a reaction resulting in production of detectable levels of light. Such a method allows determination of the identity of a specific nucleotide adjacent to the site of hybridization of the primer.

The presence of a non-mutated nucleic sequence may also be detected using combined bisulfite restriction analysis (COBRA) essentially as described in Xiong and Laird, Nucl Acids Res., 25:2532-2534, 2001. This method exploits the differences in restriction enzyme recognition sites between methylated and unmethylated nucleic acid after bisulfite treatment. Methylation specific microarrays (MSO) are also useful for differentiating between a mutated and non-mutated sequence. A suitable method is described, for example, in Adorjin et al, Nucl. Acids Res., 30: e21, 2002.

In other examples, the cfDNA may be used as a template directly in a methylation-sensitive PCR assay. Methylation-sensitive PCR may rely upon the use of a methylation-sensitive restriction enzyme which cuts either methylated or unmethylated DNA but not both. Exemplary methylation-sensitive restriction enzymes include Aatll, Aval, Cfol, Eco471III, Hpal, Hpall, Mlul, Nael, Narl, Notl, Nrul, Pvul, Sacll, Smal, SnaBI and Xhol. Hpal, for example, recognises and cuts GTT|AAC sites when unmethylated. Hpall does not cut its CCGG recognition site when it is methylated. DNA may be treated with a methylation-sensitive restriction enzyme and subsequently used as a template for PCR amplification using primers flanking the recognition and cleavage site of the methylation-sensitive restriction enzyme. The PCR assay may be quantitative or semi-quantitative.

U.S. Pat. No. 7,229,759 also describes a technology (sometimes referred to as “methylight”) that may be used to detect methylation status.

In some examples, the method of detecting methylation status does not involve genome sequencing. In some examples, the method does not involve DNA sequencing. PCR-based assays may be cheaper and faster than sequencing-based methods. Moreover, certain biological samples such as urine may be better suited to PCR-based techniques rather than sequencing-based methods due to issues such as cfDNA fragmentation.

Methods for designing probes and/or primers for use in, for example, PCR or hybridisation are known in the art and described, for example, in Dieffenbach and Dveksler (Eds) (In: PCR Primer: A Laboratory Manual, Cold Spring Harbor Laboratories, NY, 1995). Furthermore, several software packages are publicly available that design probes and/or primers for a variety of assays. In some examples, the primers and/or probes comprise fluorescent labels. The fluorescent signal from the probe may be measured as the readout and tissue composition of the cfDNA may be inferred from the readout.

Preferably, the epigenetic marker used in the methods of the present disclosure is DNA methylation status within one or more DMRs of the cfDNA. In some examples, the epigenetic marker is kidney-specific. Kidney-specific epigenetic markers may include methylated DMRs at one or more loci selected from the group consisting of GRAMD1B, DDC, MAST4, MCF2L, PAX2, chr12-122277360 (CLIP1), chr17-35303285, DEF6, EMX1, HPD, PDE4D and SPAG5. In some examples, the DMRs are located at one locus selected from the group consisting of GRAMD1B, DDC, MAST4, MCF2L, PAX2, chr12-122277360 (CLIP1), chr17 35303285, DEF6, EMX1, HPD, PDE4D and SPAG5. In some examples, the DMRs are located at more than one locus selected from the group consisting of GRAMD1B, DDC, MAST4, MCF2L, PAX2, chr12-122277360 (CLIP1), chr17 35303285, DEF6, EMX1, HPD, PDE4D and SPAG5.

In some examples, the DMRs are located at one or more loci selected from the group consisting of GRAMD1B, DDC, MAST4, MCF2L and PAX2. Sequences from these loci are set forth in Table 12, but the skilled person will understand that natural polymorphisms and allelic variation will exist between individuals. In some examples, the DMRs are located at one locus selected from the group consisting of GRAMD1B, DDC, MAST4, MCF2L and PAX2. In some examples, at least one DMR is located in PAX2. In some examples, at least one DMR comprises the sequence set forth in SEQ ID NO. 1 or SEQ ID NO. 2 or a sequence having at least 90% identity to SEQ ID NO. 1 or SEQ ID NO. 2. In some examples, at least one DMR is located in GRAMD1B. In some examples, at least one DMR comprises the sequence set forth in SEQ ID NO. 8 or SEQ ID NO. 9 or a sequence having at least 90% identity to SEQ ID NO. 8 or SEQ ID NO. 9. In some examples, at least one DMR is located in DDC. In some examples, at least one DMR comprises the sequence set forth in SEQ ID NO. 15 or SEQ ID NO. 16 or a sequence having at least 90% identity SEQ ID NO. 15 or SEQ ID NO. 16. In some examples, at least one DMR is located in MAST4. In some examples, at least one DMR comprises the sequence set forth in SEQ ID NO. 22 or SEQ ID NO. 23 or a sequence having at least 90% identity to SEQ ID NO. 22 or SEQ ID NO. 23. In some examples, at least one DMR is located in MCF2L. In some examples, at least one DMR comprises the sequence set forth in SEQ ID NO. 29 or SEQ ID NO. 30 or a sequence having at least 90% identity to SEQ ID NO. 29 or SEQ ID NO. 30.

In some examples, the DMRs are located at two loci selected from the group consisting of GRAMD1B, DDC, MAST4, MCF2L and PAX2. For example, the DMRs may be located at: GRAMD1B and DDC; GRAMD1B and MAST4; GRAMD1B and MCF2L; GRAMD1B and PAX2; DDC and MAST4; DDC and MCF2L; DDC and PAX2; MAST4 and MCF2L; MAST4 and PAX2; or MCF2L and PAX2. In some examples, the DMRs are located at three loci selected from the group consisting of GRAMD1B, DDC, MAST4, MCF2L and PAX2. For example, the DMRs may be located at: GRAMD1B, DDC and MAST4; GRAMD1B, DDC and MCF2L; GRAMD1B, DDC and PAX2; GRAMD1B, MAST4 and MCF2L; GRAMD1B, MAST4 and PAX2; GRAMD1B, MCF2L and PAX2; DDC, MAST4 and MCF2L; DDC, MAST4 and PAX2; DDC, MCF2L and PAX2; or MAST4; MCF2L and PAX2. In some examples, the DMRs are located at four loci selected from the group consisting of GRAMD1B, DDC, MAST4, MCF2L and PAX2. For example, the DMRs may be located at GRAMD1B, DDC, MAST4 and MCF2L; GRAMD1B, DDC, MAST4 and PAX2; GRAMD1B, MAST4, MCF2L and PAX2; GRAMD1B, DDC, MCF2L and PAX2; or DDC, MAST4, MCF2L and PAX2. In some examples, the DMRs are located at GRAMD1B, DDC, MAST4, MCF2L and PAX2.

In examples where the DMRs are located at more than one loci, the methylation status at each locus may be detected in separate, singleplex assays, or the methylation status at all loci may be detected in a single, multiplex assay. For example, the DMRs may be located at two loci and the methylation status may be detected using a duplex assay. In another example, the DMRs are located at three loci and the methylation status may be detected using a triplex assay. The method may, for example, comprise detecting the methylation status at GRAMD1B, DDC and PAX2 using a triplex assay. In another example, the DMRs are located at four loci and the methylation status may be detected using a quadruplex assay. In another example, the DMRs are located at five loci and the methylation status may be detected using a pentaplex assay.

A DMR may comprise one methylation site or multiple methylation sites. DMRs may be adjacent to each other on the same chromosome, or they may be located distally apart on a chromosome or on different chromosomes.

The present disclosure also provides isolated nucleic acids corresponding to a tissue- or organ-specific DMR, optionally wherein the nucleic acid is bisulfite-treated. In one example, the present disclosure provides an isolated nucleic acid having a sequence derived from or corresponding to GRAMD1B, DDC, MAST4, MCF2L or PAX2, or a portion of GRAMD1B, DDC, MAST4, MCF2L or PAX2. The portion is preferably at least 30 nucleotides in length, such as between about 30 nucleotides and 600 nucleotides, or between about 30 nucleotides and 500 nucleotides, or between about 30 nucleotides and 400 nucleotides, or between about 30 nucleotides and 350 nucleotides, or between about 30 nucleotides and 300 nucleotides, or between about 30 nucleotides and 250 nucleotides, or between about 30 nucleotides and 200 nucleotides, or between about 30 nucleotides and 150 nucleotides, or between about 40 nucleotides and 150 nucleotides, or between about 40 nucleotides and 100 nucleotides, or between about 50 nucleotides and 100 nucleotides in length. In some examples, the present disclosure provides an isolated nucleic acid having a sequence that is at least 80% identical, or at least 85% identical, or at least 90% identical, or at least 95% identical or 100% identical to the sequence set forth in any one of SEQ ID NOs. 1 to 35. In some examples, the present disclosure provides an isolated nucleic acid produced by bisulfite-treatment of a nucleic acid molecule having a sequence that is at least 80% identical, or at least 85% identical, or at least 90% identical, or at least 95% identical or 100% identical to the sequence set forth in SEQ ID NO. 1, SEQ ID NO. 2, SEQ ID NO. 8, SEQ ID NO. 9, SEQ ID NO. 15, SEQ ID NO. 16, SEQ ID NO. 22, SEQ ID NO. 23, SEQ ID NO. 29 or SEQ ID NO. 30.

In some examples, the nucleic acid is bisulfite-treated. In some examples, the present disclosure provides a bisulfite-treated nucleic acid having a sequence that is at least 80% identical, or at least 85% identical, or at least 90% identical, or at least 95% identical or 100% identical to the sequence set forth in SEQ ID NO. 3, SEQ ID NO. 4, SEQ ID NO. 5, SEQ ID NO. 6, SEQ ID NO. 7, SEQ ID NO. 10, SEQ ID NO. 11, SEQ ID NO. 12, SEQ ID NO. 13, SEQ ID NO. 14, SEQ ID NO. 17, SEQ ID NO. 18, SEQ ID NO. 19, SEQ ID NO. 20, SEQ ID NO. 21, SEQ ID NO. 24, SEQ ID NO. 25, SEQ ID NO. 26, SEQ ID NO. 27, SEQ ID NO. 28, SEQ ID NO. 31, SEQ ID NO. 32, SEQ ID NO. 33, SEQ ID NO. 34 or SEQ ID NO. 35. In some examples, the bisulfite-treated nucleic acid is at least 30 nucleotides in length, such as between about 30 nucleotides and 600 nucleotides, or between about 30 nucleotides and 500 nucleotides, or between about 30 nucleotides and 400 nucleotides, or between about 30 nucleotides and 350 nucleotides, or between about 30 nucleotides and 300 nucleotides, or between about 30 nucleotides and 250 nucleotides, or between about 30 nucleotides and 200 nucleotides, or between about 30 nucleotides and 150 nucleotides, or between about 40 nucleotides and 150 nucleotides, or between about 40 nucleotides and 100 nucleotides, or between about 50 nucleotides and 100 nucleotides in length.

### Sequences

Sequences relevant to the present disclosure, including those referred to in the Examples are listed in Table 12.

TABLE 12

| SEQ | | |
|---|---|---|
| ID | | |
| NO. | Description | Sequence |
| 1 | Native sequence of PAX2 PCR assay | tcagcgcggaccgcagcgcggcccagc |
| | region (top strand) | cccgggcacccgcctcggacgctcggg |
| | | cgcca |
| 2 | Native sequence of PAX2 PCR assay | tggcgcccgagcgtccgaggcgggtgc |
| | region (bottom strand) | ccggggctgggccgcgctgcggtccgc |
| | | gctga |
| 3 | Bisulfite converted sequence of | ttagcgcggatcgtagcgcggtttagt |
| | methylated top strand of PAX2 PCR | ttcgggtattcgtttcggacgttcggg |
| | assay region (ie, bisulfite conversion | cgtta |
| | of SEQ ID NO. 1) | |
| | Forward primer sequence underlined | |
| 4 | PCR-generated complementary strand of | taacgcccgaacgtccgaaacgaatac |
| | bisulfite converted PAX2 sequence (ie, | ccgaaactaaaccgcgctacgatccgc |
| | complementary strand of SEQ ID NO. 3) | gctaa |
| | Reverse primer sequence underlined | |
| | Oligonucleotide probe in bold | |
| 5 | PAX2 forward primer | ttagcgcggatcgtagc |
| | Detecting the bisulfite and methylated | |
| | region residing Chr10 (+): | |
| | 102,587,777-102,587,793 | |
| 6 | PAX2 reverse primer | taacgcccgaacgtcc |
| | Detecting the bisulfite and methylated | |
| | region residing Chr10 (−): | |
| | 102,587,820-102,587,835 | |
| 7 | PAX2 oligonucleotide probe | aaacgaatacccgaaactaaaccgc |
| | Detecting the bisulfite and methylated | |
| | region residing Chr10 (−): | |
| | 102,587,794-102,587,818 | |
| 8 | Native sequence of GRAMD1B PCR | agacgccagcaagcgaggaagcgcagc |
| | assay region (top strand) | ggaagaaaaacaagcgggcgcgcgagg |
| | | ggagccccaggag |
| 9 | Native sequence of GRAMD1B PCR | ctcctggggctcccctcgcgcgcccgc |
| | assay region (bottom strand) | ttgtttttcttccgctgcgcttcctcg |
| | | cttgctggcgtct |
| 10 | Bisulfite converted sequence of | agacgttagtaagcgaggaagcgtagc |
| | methylated top strand of GRAMD1B PCR | ggaagaaaaataagcgggcgcgcgagg |
| | assay region (ie, bisulfite conversion | ggagttttaggag |
| | of SEQ ID NO. 8) | |
| | Forward primer sequence underlined | |
| 11 | PCR-generated complementary strand of | ctcctaaaactcccctcgcgcgcccgc |
| | bisulfite converted GRAMD1B sequence | ttatttttcttccgctacgcttcctcg |
| | (ie, complementary strand of SEQ ID | cttactaacgtct |
| | NO. 10) | |
| | Reverse primer sequence underlined | |
| | Oligonucleotide probe in bold | |
| 12 | GRAMD1B forward primer | agacgttagtaagcgaggaagc |
| | Detecting the bisulfite and methylated | |
| | region residing Chr11 (+): | |
| | 123,301,149-123,301,170 | |
| 13 | GRAMD1B reverse primer | ctcctaaaactcccctcgc |
| | Detecting the bisulfite and methylated | |
| | region residing Chr11 (−): | |
| | 123,301,198-123,301,216 | |
| 14 | GRAMD1B oligonucleotide probe | cccgcttatttttcttccgctac |
| | Detecting the bisulfite and methylated | |
| | region residing Chr11 (−): | |
| | 123,301,171-123,301,194 | |
| 15 | Native sequence of DDC PCR assay | cgtgccttgctcattctaagcccgcgg |
| | region (top strand) | atacagcagggcccccatgttgcaggt |
| | | gctggggaggacctgcggaaacgcggc |
| | | gcc |
| 16 | Native sequence of DDC PCR assay | ggcgccgcgtttccgcaggtcctcccc |
| | region (bottom strand) | agcacctgcaacatgggggccctgctg |
| | | tatccgcgggcttagaatgagcaaggc |
| | | acgggcc |
| 17 | Bisulfite converted sequence of | cgtgttttgtttattttaagttcgcgg |
| | methylated top strand of DDC PCR assay | atatagtagggtttttatgttgtaggt |
| | region (ie, bisulfite conversion of | gttggggaggatttgcggaaacgcggc |
| | SEQ ID NO. 15) | gtt |
| | Forward primer sequence underlined | |
| 18 | PCR-generated complementary strand of | aacgccgcgtttccgcaaatcctcccc |
| | bisulfite converted DDC sequence (ie, | aacacctacaacataaaaaccctacta |
| | complementary strand of SEQ ID NO. 17) | tatccgcgaacttaaaataaacaaaac |
| | Reverse primer sequence underlined | acg |
| | Oligonucleotide probe in bold | |
| 19 | DDC forward primer | cgtgttttgtttattttaagttcgc |
| | Detecting the bisulfite and methylated | |
| | region residing Chr7 (+): | |
| | 50,535,754-50,535,778 | |
| 20 | DDC reverse primer | aacgccgcgtttccg |
| | Detecting the bisulfite and methylated | |
| | region residing Chr7 (−): | |
| | 50,535,823-50,535,837 | |
| 21 | DDC oligonucleotide probe | cctacaacataaaaaccctactatatc |
| | Detecting the bisulfite and methylated | cgcg |
| | region residing Chr7 (−): | |
| | 50,535,776-50,535,806 | |
| 22 | Native sequence of MAST4 PCR assay | gacggatccattccgggctccggggag |
| | region (top strand) | ggggagcggcggcccaggctgcggggc |
| | | tgtcccggcaccg |
| 23 | Native sequence of MAST4 PCR assay | cggtgccgggacagccccgcagcctgg |
| | region (bottom strand) | gccgccgctccccctccccggagcccg |
| | | gaatggatccgtc |
| 24 | Bisulfite converted sequence of | gacggatttatttcgggtttcggggag |
| | methylated top strand of MAST4 PCR | ggggagcggcggtttaggttgcggggt |
| | assay region (ie, bisulfite conversion | tgtttcggtatcg |
| | of SEQ ID NO. 22) | |
| | Forward primer sequence underlined | |
| 25 | PCR-generated complementary strand of | cgataccgaaacaaccccgcaacctaa |
| | bisulfite converted MAST4 sequence | accgccgctccccctccccgaaacccg |
| | (ie, complementary strand of SEQ ID | aaataaatccgtc |
| | NO. 24) | |
| | Reverse primer sequence underlined | |
| | Oligonucleotide probe in bold | |
| 26 | MAST4 forward primer | gacggatttatttcgggtttc |
| | Detecting the bisulfite and methylated | |
| | region residing Chr11 (+): | |
| | 66,299,953-66,299,973 | |
| 27 | MAST4 reverse primer | cgataccgaaacaacccc |
| | Detecting the bisulfite and methylated | |
| | region residing Chr11 (−): | |
| | 66,300,002-66,300,019 | |
| 28 | MAST4 oligonucleotide probe | ctaaaccgccgctccccc |
| | Detecting the bisulfite and methylated | |
| | region residing Chr11 (−): | |
| | 66,299,979-66,299,996 | |
| 29 | Native sequence of MCF2L PCR assay | cgctgccgtggccccctccccgcctcc |
| | region (top strand) | gccgcgccccctccgcactcgcacggc |
| | | cccacccgcaggcgcccccc |
| 30 | Native sequence of MCF2L PCR assay | ggggggcgcctgcgggtggggccgtgc |
| | region (bottom strand) | gagtgcggagggggcgcggcggaggcg |
| | | gggagggggccacggcagcg |
| 31 | Bisulfite converted sequence of | cgttgtcgtggtttttttttcgttttc |
| | methylated top strand of MCF2L PCR | gtcgcgtttttttcgtattcgtacggt |
| | assay region (ie, bisulfite conversion | tttattcgtaggcgtttttc |
| | of SEQ ID NO. 29) | |
| | Forward primer sequence underlined | |
| 32 | PCR-generated complementary strand of | gaaaaacgcctacgaataaaaccgtac |
| | bisulfite converted MCF2L sequence | gaatacgaaaaaaacgcgacgaaaacg |
| | (ie, complementary strand of SEQ ID | aaaaaaaaaccacgacaacg |
| | NO. 31) | |
| | Reverse primer sequence underlined | |
| | Oligonucleotide probe in bold | |
| 33 | MCF2L forward primer | cgttgtcgtggtttttttttc |
| | Detecting the bisulfite and methylated | |
| | region residing Chr13 (+): | |
| | 113,623,573-113,623,593 | |
| 34 | MCF2L reverse primer | gaaaaacgcctacgaataaaacc |
| | Detecting the bisulfite and methylated | |
| | region residing Chr13 (−): | |
| | 113,623,624-113,623,646 | |
| 35 | MCF2L oligonucleotide probe | tacgaatacgaaaaaaacgcgacg |
| | Detecting the bisulfite and methylated | |
| | region residing Chr13 (−): | |
| | 113,623,599-113,623,622 | |
| 36 | ACTB PCR assay region (top strand) | ctcagccaatgggacctgctcctccct |
| | Reverse primer underlined | tgaaggttgcagaggccacagcctggt |
| | | gggaaagatgaccaccacccagcacac |
| | | agtggcagacacaggttcacagtccag |
| | | aagacttgctgagcctcctccatcac |
| 37 | ACTB PCR assay region (bottom strand) | gtgatggaggaggctcagcaagtcttc |
| | Forward primer underlined | tggactgtgaacctgtgtctgccactg |
| | Oligonucleotide probe underlined | tgtgctgggtggtggtcatctttccca |
| | | ccaggctgtggcctctgcaaccttcaa |
| | | gggaggagcaggtcccattggctgag |
| 38 | ACTB forward primer | ggaggaggctcagcaagtc |
| | Detecting the genomic region residing | |
| | Chr7 (+): 5,571,836-5,571,854 | |
| 39 | ACTB reverse primer | ctcagccaatgggacctg |
| | Detecting the genomic region residing | |
| | Chr7 (−): 5,571,726-5,571,743 | |
| 40 | ACTB oligonucleotide probe | acctgtgtctgccactgtgtgct |
| | Detecting the genomic region residing | |
| | Chr7 (+): 5,571,800-5,571,822 | |
| 41 | ACTB bisulfite PCR top (complementary) | caaaataaaatacaaaacaaacctaat |
| | strand | ccccaaacaaaacctatattaccacta |
| | | aacctccattcaactaaccaaaaaaca |
| | | aaaactcc |
| 42 | ACTB bisulfite converted PCR bottom | ggagtttttgttttttggttagttgaa |
| | strand | tggaggtttagtggtaatataggtttt |
| | | gtttggggattaggtttgttttgtatt |
| | | ttattttg |
| 43 | ACTB bisulfite forward primer | ggagtttttttttttggttagttg |
| | Detecting the bisulfite and methylated | |
| | region residing Chr7 (+): | |
| | 5,572,241-5,572,265 | |
| 44 | ACTB bisulfite reverse primer | caaaataaaatacaaaacaaacctaat |
| | Detecting the bisulfite and methylated | Cc |
| | region residing Chr7 (−): | |
| | 5,572,176-5,572,204 | |
| 45 | ACTB bisulfite oligonucleotide probe | atggaggtttagtggtaatataggttt |
| | Detecting the bisulfite and methylated | tgtttgg |
| | region residing Chr7 (+): | |
| | 5,572,206-5,572,239 | |
| 46 | Native sequence of Feline PAX2 PCR | acaatcagcgcggactgcagcgcggcc |
| | assay region 1 (top strand) | cagccccggagcagcccgcctcggacg |
| | | ctccggcgcct |
| 47 | Native sequence of Feline PAX2 PCR | aggcgccggagcgtccgaggcgggctg |
| | assay region 1 (bottom strand) | ctccggggctgggccgcgctgcagtcc |
| | | gcgctgattgt |
| 48 | Bisulfite converted sequence of | ataattagcgcggattgtagcgcggtt |
| | methylated top strand of Feline PAX2 | tagtttcggagtagttcgtttcggacg |
| | PCR assay region 1 (ie, bisulfite | tttcggcgttt |
| | conversion of SEQ ID NO. 46) | |
| | Forward primer sequence underlined | |
| 49 | PCR-generated complementary strand of | aaacgccgaaacgtccgaaacgaacta |
| | bisulfite converted Feline PAX2 | ctccgaaactaaaccgcgctacaatcc |
| | sequence 1 (ie, complementary strand | gcgctaattat |
| | of SEQ ID NO. 48) | |
| | Reverse primer sequence underlined | |
| | Oligonucleotide probe in bold | |
| 50 | Feline PAX2 forward primer 1 | ataattagcgcggattgtagc |
| | Detecting the bisulfite and methylated | |
| | region residing ChrD2 (+): | |
| | 61,508,592-61,508,612 | |
| 51 | Feline PAX2 reverse primer 1 | aaacgccgaaacgtcc |
| | Detecting the bisulfite and methylated | |
| | region residing ChrD2 (−): | |
| | 61,508,656-61,508,640 | |
| 52 | Feline PAX2 oligonucleotide probe 1 | aaacgaactactccgaaactaaaccgc |
| | Detecting the bisulfite and methylated | |
| | region residing ChrD2 (−): | |
| | 61,508,639-61,508,613 | |
| 53 | Native sequence of Feline PAX2 PCR | ctgcagcgcggcccagccccggagcag |
| | assay region 2 (top strand) | cccgcctcggacgctccggcgcctgga |
| | | ggcttcgctggt |
| 54 | Native sequence of Feline PAX2 PCR | accagcgaagcctccaggcgccggagc |
| | assay region 2 (bottom strand) | gtccgaggcgggctgctccggggctgg |
| | | gccgcgctgcag |
| 55 | Bisulfite converted sequence of | ttgtagcgcggtttagtttcggagtag |
| | methylated top strand of Feline PAX2 | ttcgtttcggacgtttcggcgtttgga |
| | PCR assay region 2 (ie, bisulfite | ggtttcgttggt |
| | conversion of SEQ ID NO. 53) | |
| | Forward primer sequence underlined | |
| 56 | PCR-generated complementary strand of | accaacgaaacctccaaacgccgaaac |
| | bisulfite converted Feline PAX2 | gtccgaaacgaactactccgaaactaa |
| | sequence 2 (ie, complementary strand | accgcgctacaa |
| | of SEQ ID NO. 55) | |
| | Reverse primer sequence underlined | |
| | Oligonucleotide probe in bold | |
| 57 | Feline PAX2 forward primer 2 | ttgtagcgcggtttagtttc |
| | Detecting the bisulfite and methylated | |
| | region residing ChrD2 (+): | |
| | 61,508,606-61,508,625 | |
| 58 | Feline PAX2 reverse primer 2 | accaacgaaacctccaaac |
| | Detecting the bisulfite and methylated | |
| | region residing ChrD2 (−): | |
| | 61,508,669-61,508,653 | |
| 59 | Feline PAX2 oligonucleotide probe 2 | ccgaaacgtccgaaacgaactactcc |
| | Detecting the bisulfite and methylated | |
| | region residing ChrD2 (−): | |
| | 61,508,651-61,508,626 | |
| 60 | Native sequence of Canine PAX2 PCR | ccgcagcgcggcccagcccgggagcag |
| | assay region (top strand) | cccgcctcggacgctccggcgccggga |
| | | ggcttcgctg |
| 61 | Native sequence of Canine PAX2 PCR | cagcgaagcctcccggcgccggagcgt |
| | assay region (bottom strand) | ccgaggcgggctgctcccgggctgggc |
| | | cgcgctgcgg |
| 62 | Bisulfite converted sequence of | tcgtagcgcggtttagttcgggagtag |
| | methylated top strand of Canine PAX2 | ttcgtttcggacgtttcggcgtcggga |
| | PCR assay region (ie, bisulfite | ggtttcgttg |
| | conversion of SEQ ID NO. 60) | |
| | Forward primer sequence underlined | |
| 63 | PCR-generated complementary strand of | caacgaaacctcccgacgccgaaacgt |
| | bisulfite converted Canine PAX2 | ccgaaacgaactactcccgaactaaac |
| | sequence (ie, complementary strand of | cgcgctacga |
| | SEQ ID NO. 62) | |
| | Reverse primer sequence underlined | |
| | Oligonucleotide probe in bold | |
| 64 | Canine PAX2 forward primer | tcgtagcgcggtttagttc |
| | Detecting the bisulfite and methylated | |
| | region residing Chr28 (+): | |
| | 13,710,063-13,710,081 | |
| 65 | Canine PAX2 reverse primer | caacgaaacctcccgac |
| | Detecting the bisulfite and methylated | |
| | region residing Chr28 (−): | |
| | 13,710,126-13,710,109 | |
| 66 | Canine PAX2 oligonucleotide probe | ccgaaacgtccgaaacgaactactcc |
| | Detecting the bisulfite and methylated | |
| | region residing Chr28 (−): | |
| | 13,710,107-13,710,083 | |

### Indications of Cell Damage

An assessment as to whether the subject is suffering from, or at risk of, tissue or organ damage may be made by comparing the level of the epigenetic marker to a reference level, or by monitoring the level of the epigenetic marker over time. A reference level of the epigenetic marker in cfDNA may be used as a baseline against which the level of the epigenetic marker in a cfDNA sample is compared. The reference level may represent the concentration of the epigenetic marker that is expected in the cfDNA of a healthy individual or a group or population of healthy individuals. A higher or lower concentration of the epigenetic marker in the sample cfDNA relative to the reference level may indicate that the subject is suffering from, or at risk of, tissue or organ damage. The reference level may be based on studies conducted on cfDNA taken from healthy individuals, or it may be based on the concentration of the epigenetic marker in cfDNA from the subject at a defined point in time (eg, prior to a particular treatment). The reference level may be based on a data set comprising levels of the epigenetic marker in a healthy subject or a population of healthy individuals.

A difference of at least about 5% in the level of the epigenetic marker in the sample cfDNA compared to the reference level may be indicative of tissue or organ damage. For example, a difference of at least about 10%, such as at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or at least about 100% may be indicative of tissue or organ damage. In some examples, organ or tissue damage may be indicated when the amount of the epigenetic marker present in cfDNA of the sample is about 5% higher than the amount present in the reference cfDNA. In some examples, organ or tissue damage may be indicated when the amount of the epigenetic marker present in cfDNA of the sample is at least about 10% higher, such as at least about 15% higher, at least about 20% higher, at least about 25% higher, at least about 30% higher, at least about 35% higher, at least about 40% higher, at least about 45% higher, at least about 50% higher, at least about 55% higher, at least about 60% higher, at least about 65% higher, at least about 70% higher, at least about 75% higher, at least about 80% higher, at least about 85% higher, at least about 90% higher, at least about 95% higher or at least about 100% higher than the amount present in the reference cfDNA. In some examples, organ or tissue damage may be indicated when the amount of the epigenetic marker present in cfDNA of the sample is at least about 2-fold higher, such as at least about 3-fold higher, at least about 4-fold higher, at least about 5-fold higher, at least about 6-fold higher, at least about 7-fold higher, at least about 8-fold higher, at least about 9-fold higher or at least about 10-fold higher than the amount present in the reference cfDNA.

In some examples, the reference level of the epigenetic marker may be zero (not detectable) and the detectable presence of the epigenetic marker in the sample cfDNA may be indicative of tissue or organ damage. In other examples, the level of the epigenetic marker is measured over time. A subject may be monitored, for example, by taking biological samples from the subject over time and measuring the level of the epigenetic marker in cfDNA from each biological sample. An increase in the concentration of the epigenetic marker in cfDNA over time may indicate that the subject is suffering from, or at risk of, tissue or organ damage.

The methods described herein may be performed on a subject of any age, but cell damage may be more prevalent in older subjects compared to younger subjects. Correspondingly, cell-, tissue- or organ-specific epigenetic markers may be present at higher concentrations in older subjects relative to younger subjects. In some examples, the present disclosure provides a method of detecting organ damage in a subject, wherein the subject is at least 5 years old. In some examples, the subject is at least 10 years old, or at least 15 years old, or at least 20 years old, or at least 25 years old, or at least 30 years old, or at least 35 years old, or at least 40 years old, or at least 45 years old, or at least 50 years old, or at least 55 years old, or at least 60 years old, or at least 65 years old.

Epigenetic data can be combined and made more clinically useful through the use of various formulae, including statistical classification algorithms and others, combining and in many cases extending the performance characteristics of the combination beyond that of any individual data point. These specific combinations show an acceptable level of diagnostic/prognostic accuracy, and, when sufficient information from one or more markers is combined in a trained formula, may reliably achieve a high level of diagnostic/prognostic accuracy transportable from one population to another.

Several statistical and modelling algorithms known in the art can be used to assist in marker selection and to optimise the algorithms combining these selections. Statistical tools such as factor and cross-marker correlation/covariance analyses may allow more rational approaches to panel construction. Mathematical clustering and classification tree showing the Euclidean standardised distance between the markers may be advantageously used. Pathway informed seeding of such statistical classification techniques also may be employed, as may rational approaches based on the selection of individual markers and their participation across particular pathways or physiological functions or individual performance.

Formulae such as statistical classification algorithms may be directly used to both select epigenetic markers and to generate and train the formula to combine the results from multiple epigenetic markers into a single index. Often techniques such as forward (from zero potential explanatory parameters) and backwards selection (from all available potential explanatory parameters) are used, and information criteria are used to quantify the trade-off between the performance and diagnostic/prognostic accuracy of the panel and the number of epigenetic markers used. The position of the individual epigenetic markers on a forward or backwards selected panel can be closely related to its provision of incremental information content for the algorithm, so the order of contribution may be dependent on the other constituent markers in the panel.

Any suitable formula may be used to combine epigenetic marker results into indices or indexes useful in the methods of the disclosure. As indicated herein, and without limitation, such indices may indicate, among the various other indications, the probability, likelihood, absolute or relative risk, time to or rate of organ damage, conversion from one to another disease states, or make predictions of future epigenetic marker measurements of organ or tissue damage. This may be for a specific time period or horizon, or for remaining lifetime risk, or simply be provided as an index relative to another reference subject population.

The actual model type or formula used may itself be selected from the field of potential models based on the performance and diagnostic accuracy characteristics of its results in a training population. The specifics of the formula itself may commonly be derived from marker results in the relevant training population. Amongst other uses, such formula may be intended to map the feature space derived from one or more marker inputs to a set of subject classes (eg, useful in predicting class membership of subjects as normal, at risk of organ damage, or responding/not-responding to treatment), to derive an estimation of a probability function of risk using a Bayesian approach (eg, the risk of organ damage or recurrence event), or to estimate the class-conditional probabilities, then use Bayes' rule to produce the class probability function.

Following analysis and determination of an index of probability of the presence or absence of organ damage, or response to treatment, the index can be transmitted or provided to a third party, e.g., a medical practitioner for assessment. The index may be used by the practitioner to assess whether or not additional diagnostic methods are required, e.g., biopsy and histological analysis and/or other assays, or a change in treatment or commencement of treatment.

Knowledge-based computer software and hardware for implementing an algorithm of the disclosure also form part of the present disclosure. Thus, the present disclosure also provides software or hardware programmed to implement an algorithm that processes data obtained by performing the method of the disclosure via a univariate or multivariate analysis to provide a damage index value and provide or permit a diagnosis of organ or tissue damage.

In one example, a method of the disclosure may be used in existing knowledge-based architecture or platforms associated with pathology services. For example, results from a method described herein are transmitted via a communications network (eg, the internet) to a processing system in which an algorithm is stored and used to generate a predicted posterior probability value which translates to the index of damage probability which is then forwarded to an end user in the form of a diagnostic or predictive report. The method of the disclosure may, therefore, be in the form of a kit or computer-based system which comprises the reagents necessary to detect the level of the epigenetic marker(s) and the computer hardware and/or software to facilitate determination and transmission of reports to a clinician.

In some examples, the present disclosure permits integration of an assay into existing or specifically developed pathology architecture or platform systems. For example, the present disclosure contemplates a method of allowing a user to determine the status of a subject with respect to organ damage, the method comprising: (a) receiving sample epigenetic data in the form of a level of an organ-specific epigenetic marker in cfDNA obtained from a biological sample of the subject relative to reference epigenetic data, optionally in combination with another marker of organ damage; (b) processing the sample epigenetic data via univariate and/or multivariate analysis to provide a damage index value; (c) determining the status of the subject in accordance with the damage index value in comparison with predetermined values; and (d) transferring an indication of the status of the subject to the user via a communications network.

In some examples, the method further comprises: (i) having the user determine the data using a remote end station; and (ii) transferring the data from the end station to a base station via the communications network. The base station may include first and second processing systems, in which case the method may comprise: (a) transferring the data to the first processing system; (b) transferring the data to the second processing system; and (c) causing the first processing system to perform the univariate or multivariate analysis to generate the damage index value.

The method may also comprise: (a) transferring the results of the univariate or multivariate analysis function to the first processing system; and (b) causing the first processing system to determine the status of the subject.

Biological samples from which cfDNA may be obtained may include saliva, blood or serum or plasma, urine, semen, vitreous fluid, lymph fluid, synovial fluid, follicular fluid, gastric fluid, intestinal fluid, bile, tumour fluid, interstitial fluid, amniotic fluid, mucus, breast milk, pleural fluid, sweat, tears, stool, serum or cerebro-spinal fluid. Those skilled in the art will appreciate that other biological samples may be taken as a source of cfDNA. Methods for obtaining a biological sample from a subject are known in the art and include, for example, surgery, biopsy or collection of a bodily fluid, for example, by paracentesis or thoracentesis or collection of, for example, blood or a fraction thereof. In some examples, the methods of the present disclosure include obtaining a biological sample comprising cfDNA from a subject, and optionally isolating the cfDNA from the biological sample.

Preferably, the biological sample is a liquid. Some biological samples may be better suited than others for detecting organ or tissue damage depending upon the particular organ or tissue in question. In some examples, urine is used as a biological sample to detect kidney damage. In other examples, blood or plasma is used as a biological sample to detect heart damage.

The DMR may be present in a biological sample of a healthy subject at a concentration of at least about 3.3 pg of single stranded DNA/mL, such as at least about 4 μg/mL, or at least about 5 μg/mL, or at least about 10 μg/mL, or at least about 20 μg/mL, or at least about 30 μg/mL, or at least about 40 pg/mL, or at least about 50 μg/mL, or at least 75 μg/mL, or at least about 100 μg/mL, or at least about 125 μg/mL, or at least about 150 μg/mL, or at least about 175 μg/mL, or at least about 200 μg/mL, or at least about 225 μg/mL, or at least about 250 μg/mL, or at least about 275 μg/mL, or at least about 300 μg/mL, or at least about 325 μg/mL, or at least about 350 μg/mL, or at least about 375 μg/mL, or at least about 400 μg/mL, or at least about 425 μg/mL, or at least about 450 μg/mL, or at least about 475 μg/mL, or at least about 500 μg/mL, or at least about 525 μg/mL, or at least about 550 μg/mL, or at least about 575 μg/mL, or at least about 600 μg/mL, or at least about 625 μg/mL, or at least about 650 μg/mL. The DMR may be present in a biological sample of a healthy subject at a concentration of at least about 1 copy/mL, such as at least about 5 copies/mL, or at least about 10 copies/mL, or at least about 15 copies/mL, or at least about 20 copies/mL, or at least about 25 copies/mL, or at least about 50 copies/mL, or at least about 75 copies/mL, or at least about 100 copies/mL, or at least about 150 copies/mL, or at least about 200 copies/mL, or at least about 250 copies/mL, or at least about 300 copies/mL, or at least about 350 copies/mL, or at least about 400 copies/mL, or at least about 450 copies/mL, or at least about 500 copies/mL, or at least about 600 copies/mL, or at least about 700 copies/mL, or at least about 800 copies/mL, or at least about 900 copies/mL, or at least about 1000 copies/mL.

In some examples, the proportion of the cfDNA in a biological sample of a healthy subject that corresponds to the epigenetic marker is 1%; for example, in circumstances where the epigenetic marker is methylated DNA at locus A, about 1% of the locus A DNA molecules in the cfDNA are methylated. In some examples, the proportion of the cfDNA in a biological sample of a healthy subject that corresponds to the tissue- or organ-specific epigenetic marker is less than about 75%, such as less than about 70%, or less than about 65%, or less than about 60%, or less than about 55%, or less than about 50%, or less than about 45%, or less than about 40%, or less than about 35%, or less than about 30%, or less than about 25%, or less than about 20%, or less than about 15%, or less than about 10%, or less than about 5%, or less than about 1%, or less than about 0.1%. It will be understood that the proportion of the cfDNA that corresponds to the epigenetic marker may be lower where the marker is cell-type-specific or tissue-specific, and higher where the marker is organ-specific. It will also be understood that the proportion of the cfDNA that corresponds to the epigenetic marker may increase when there is tissue or organ damage.

### Methods of the Present Disclosure

Method 1. A method of diagnosing organ damage in a subject the method comprising detecting an organ-specific epigenetic marker in cfDNA obtained from a biological sample of the subject, wherein the presence of the epigenetic marker in the cfDNA is indicative of organ damage.

Method 2. A method of detecting organ damage in a subject, the method comprising: a) obtaining a biological sample comprising cfDNA from the subject; and b) detecting an organ-specific epigenetic marker in the cfDNA, wherein the presence of the epigenetic marker in the cfDNA is indicative of organ damage.

Method 3. The method of Method 1 or Method 2 wherein the method comprises detecting an increase in the level of the epigenetic marker relative to a reference level.

Method 4. The method of Method 1 or Method 2 wherein the method comprises detecting an increase in the level of the epigenetic marker over time.

Method 5. The method of any one of Methods 1 to 4 wherein the epigenetic marker is DNA methylation status at a differentially methylated region of the cfDNA.

Method 6. The method of any one of Methods 1 to 5 wherein the method comprises detecting cfDNA methylation status at more than one differentially methylated region.

Method 7. The method of Method 6 wherein the methylation status is determined at more than one differentially methylated region using a multiplex assay.

Method 8. The method of any one of Methods 5 to 7 wherein the methylation status is determined by a method that does not involve DNA sequencing.

Method 9. The method of any one of Methods 5 to 8 wherein the methylation status is determined by treating the cfDNA with bisulfite and amplifying the differentially methylated region using polymerase chain reaction (PCR).

Method 10. The method of Method 9 wherein the PCR is digital PCR (dPCR), digital droplet PCR (ddPCR) or quantitative PCR (qPCR).

Method 11. The method of any one of Methods 1 to 10 wherein the subject and the organ are autologous.

Method 12. The method of any one of Methods 1 to 11 wherein the organ is a kidney.

Method 13. The method of any one of Methods 1 to 12 wherein the organ damage is associated with acute kidney injury, chronic kidney disease or kidney transplant rejection.

Method 14. The method of any one of Method 1 to 12 wherein the organ damage is associated with chemotherapy or radiotherapy.

Method 15. The method of any one of Methods 1 to 14 wherein the biological sample is urine.

Method 16. The method of any one of Methods 5 to 15 wherein the differentially methylated region is located at one or more loci selected from the group consisting of GRAMD1B, DDC, MAST4, MCF2L, PAX2, chr12-122277360 (CLIP1), chr17-35303285, DEF6, EMX1, HPD, PDE4D and SPAG5.

Method 17. The method of any one of Methods 5 to 16 wherein the differentially methylated region is located at one or more loci selected from the group consisting of GRAMD1B, DDC, MAST4, MCF2L and PAX2.

Method 18. The method of any one of Methods 5 to 17 wherein the differentially methylated region comprises a sequence having at least 90% identity to any one or more of SEQ ID NO. 1, SEQ ID NO. 2 SEQ ID NO. 8, SEQ ID NO. 9, SEQ ID NO. 15, SEQ ID NO. 16, SEQ ID NO. 22, SEQ ID NO. 23, SEQ ID NO. 29 or SEQ ID NO. 30.

Method 19. The method of any one of Methods 1 to 18 wherein the method specifically detects damage to a defined tissue or cell-type of the organ.

Method 20. The method of Method 19 wherein the defined cell-type is renal proximal tubule epithelial cells.

Method 21. The method of Method 19 or Method 20 wherein the differentially methylated regions are located at at least one of MAST4 and DDC.

Method 22. The method of any one of Methods 1 to 21 wherein the method further comprises treating the subject for the organ damage.

Method 23. A method of diagnosing kidney damage in a subject, the method comprising detecting at least one kidney differentially methylated region in cfDNA wherein the cfDNA is obtained from a biological sample of the subject, and wherein the presence of the at least one kidney differentially methylated region in the cfDNA is indicative of kidney damage.

Method 24. A method of detecting kidney damage in a subject, the method comprising:
a) obtaining a biological sample comprising cfDNA from the subject; and
b) detecting at least one kidney differentially methylated region in the cfDNA,
   wherein the presence of the at least one kidney-specific methylation site in the cfDNA is indicative of organ damage.

Method 25. The method of method 23 or method 24 wherein the method comprises detecting an increase in the level of the at least one kidney differentially methylated region relative to a reference level.

Method 26. The method of method of method 23 or method 24 wherein the method comprises detecting an increase in the level of the at least one kidney differentially methylated region over time.

Method 27. The method of any one of methods 23 to 26 wherein the method comprises detecting cfDNA methylation status at more than one kidney differentially methylated region.

Method 28. The method of method 27 wherein the methylation status is determined at more than one kidney differentially methylated region using a multiplex assay.

Method 29. The method of any one of methods 23 to 28 wherein the methylation status is determined by a method that does not involve DNA sequencing.

Method 30. The method of any one of methods 23 to 29 wherein the methylation status is determined by treating the cfDNA with bisulfite and amplifying the at least one kidney differentially methylated region using polymerase chain reaction (PCR).

Method 31. The method of method 30 wherein the PCR is digital PCR (dPCR), digital droplet PCR (ddPCR) or quantitative PCR (qPCR).

Method 32. The method of any one of methods 23 to 31 wherein the subject and the organ are autologous.

Method 33. The method of any one of methods 23 to 32 wherein the kidney damage is associated with acute kidney injury, chronic kidney disease or kidney transplant rejection.

Method 34. The method of any one of methods 23 to 33 wherein the kidney damage is associated with chemotherapy or radiotherapy.

Method 35. The method of any one of methods 23 to 34 wherein the biological sample is urine.

Method 36. The method of any one of methods 23 to 35 wherein the at least one kidney differentially methylated region is located at one or more loci selected from the group consisting of GRAMD1B, DDC, MAST4, MCF2L, PAX2, chr12-122277360 (CLIP1), chr17-35303285, DEF6, EMX1, HPD, PDE4D and SPAG5.

Method 37. The method of any one of methods 23 to 36 wherein the at least one kidney differentially methylated region is located at one or more loci selected from the group consisting of GRAMD1B, DDC, MAST4, MCF2L and PAX2.

Method 38. The method of any one of methods 23 to 37 wherein the at least one kidney differentially methylated region comprises a sequence having at least 90% identity to any one or more of SEQ ID NO. 1, SEQ ID NO. 8, SEQ ID NO. 15, SEQ ID NO. 22 or SEQ ID NO. 29.

Method 39. The method of any one of methods 23 to 38 wherein the method specifically detects damage to a defined tissue or cell-type of the kidney.

Method 40. The method of method 39 wherein the defined cell-type is renal proximal tubule epithelial cells.

Method 41. The method of method 39 or method 40 wherein the at least one kidney differentially methylated regions are located at at least one of MAST4 and DDC.

Method 42. The method of method 39 or method 40 wherein the at least one kidney differentially methylated regions are located at at least one of GRAMD1B and DDC.

Method 43. The method of method 39 or method 40 wherein the at least one kidney differentially methylated regions are located at at least one of GRAMD1B, DDC and PAX2.

Method 44. The method of any one of methods 23 to 43 wherein the subject is a human.

Method 45. The method of any one of methods 23 to 43 wherein the subject is non-human.

Method 46. The method of method 45 wherein the subject is a domesticated animal.

Method 47. The method of method 46 wherein the domesticated animal is a companion animal.

Method 48. The method of method 47 wherein the domesticated animal is selected from the group consisting of sheep, cattle, horses, cats, dogs, pigs, and chickens.

Method 49. The method of method 47 wherein the companion animal is selected from cats and dogs.

Method 50. The method of any one of methods 23 to 43 wherein the method further comprises treating the subject for the kidney damage.

The present invention also includes the following method embodiments.

Method 51. A method of identifying at least one methylated region in cfDNA, said method comprising the steps of:
(i) obtaining cfDNA from a subject;
(ii) treating the cfDNA with bisulfite to obtain bisulfite converted cfDNA; and
(iii) identifying the at least one methylated region by PCR amplification of the bisulfite converted cfDNA with primers that selectively amplify the at least one methylated region,
   wherein the at least one methylated region is a differentially methylated region that occurs in kidney cells.

Method 52. The method of method 51, wherein the method identifies more than one differentially methylated regions.

Method 53. The method of method 51, wherein the method identifies more than one differentially methylated region using a multiplex assay.

Method 54. The method of any one of methods 51 to 53 wherein the identifying at least one methylated region in cfDNA is confirmed by DNA sequencing.

Method 55. The method of any one of methods 51 to 53 wherein the PCR is digital PCR (dPCR), digital droplet PCR (ddPCR) or quantitative PCR (qPCR).

Method 56. The method of any one of methods 51 to 55 wherein the at least one differentially methylated region is located at one or more loci selected from the group consisting of GRAMD1B, DDC, MAST4, MCF2L, PAX2, chr12-122277360, chr17-35303285, DEF6, EMX1, HPD, PDE4D and SPAG5.

Method 57. The method of any one of methods 51 to 56 wherein the at least one differentially methylated region is located at one or more loci selected from the group consisting of GRAMD1B, DDC, MAST4, MCF2L and PAX2.

Method 58. The method of any one of methods 51 to 57 wherein the at least one differentially methylated region comprises a sequence having at least 90% identity to any one or more of SEQ ID NO. 1, SEQ ID NO. 8, SEQ ID NO. 15, SEQ ID NO. 22 or SEQ ID NO. 29.

Method 59. The method of any one of methods 51 to 58 wherein the at least one differentially methylated region is differentially methylated in renal proximal tubule epithelial cells.

Method 60. The method of method 59 wherein the at least one kidney differentially methylated regions are located at at least one of MAST4 and DDC.

Method 61. The method of any one of methods 51 to 60 wherein the at least one kidney differentially methylated regions are located at at least one of GRAMD1B and DDC.

Method 62. The method of method of any one of methods 51 to 60 wherein the at least one kidney differentially methylated regions are located at at least one of GRAMD1B, DDC and PAX2 . . .

Method 63. The method of any one of methods 51 to 62 wherein the subject is a human.

Method 64. The method of any one of methods 51 to 62 wherein the subject is non-human.

Method 65. The method of method 64 wherein the subject is a domesticated animal.

Method 66. The method of method 65 wherein the domesticated animal is a companion animal.

Method 67. The method of method 65 wherein the domesticated animal is selected from the group consisting of sheep, cattle, horses, cats, dogs, pigs, and chickens.

Method 68. The method of method 47 wherein the companion animal is selected from cats and dogs.

Method 69. The method of any one of methods 23 to 43 wherein the method further comprises treating the subject for the kidney damage.

Method 70. A method of indicating to a user whether or not a subject has organ damage, the method comprising:
a) producing sample epigenetic data by determining a level of an organ-specific epigenetic marker in cell-free DNA (cfDNA) obtained from a biological sample of the subject;
b) a processor receiving the sample epigenetic data, wherein the processor also receives reference epigenetic data corresponding to the epigenetic marker;
c) the processor generating differential epigenetic data by comparing the sample epigenetic data with the reference epigenetic data;
d) the processor processing the differential epigenetic data to produce a damage index value;
e) determining by the processor a damage status of the subject based upon the damage index value, the damage status being indicative of whether or not the subject has organ damage; and
f) transferring an indication of the organ damage of the subject to the user via a communications network.

Method 71. The method of method 70 wherein the organ damage is kidney damage, and the organ-specific epigenetic marker is at least one kidney differentially methylated region, and the sample epigenetic data is sample methylation data and the reference epigenetic data is reference methylation data.

Method 72. The method of method 70 or method 71 wherein the sample methylation data is increased relative to the reference methylation data.

Method 73. The method of any one of methods 70 to 72 wherein the method comprises detecting an increase in the level of the sample methylation data over time.

Method 74. The method of any one of methods 70 to 73 wherein the sample methylation data comprises cfDNA methylation status at more than one kidney differentially methylated region.

Method 75. The method of any one of method 74 wherein the methylation status is determined at more than one kidney differentially methylated region using a multiplex assay.

Method 76. The method of any one of methods 70 to 75 wherein the methylation data is determined by treating the cfDNA with bisulfite and amplifying the at least one kidney differentially methylated region using polymerase chain reaction (PCR).

Method 77. The method of method 76 wherein the PCR is digital PCR (dPCR), digital droplet PCR (ddPCR) or quantitative PCR (qPCR).

Method 78. The method of any one of methods 70 to 77 wherein the subject and the kidney are autologous.

Method 79. The method of any one of methods 70 to 78 wherein the kidney damage is associated with acute kidney injury, chronic kidney disease or kidney transplant rejection or renal replacement therapy.

Method 80. The method of any one of methods 70 to 79 wherein the kidney damage is associated with chemotherapy or radiotherapy.

Method 81. The method of any one of methods 70 to 80 wherein biological sample is urine.

Method 82. The method of any one of methods 71 to 81 wherein the at least one kidney differentially methylated region is located at one or more loci selected from the group consisting of GRAMD1B, DDC, MAST4, MCF2L, PAX2, chr12-122277360 (CLIP1), chr17-35303285, DEF6, EMX1, HPD, PDE4D and SPAG5.

Method 82. The method of any one of methods 71 to 81 wherein the at least one kidney differentially methylated region is located at one or more loci selected from the group consisting of GRAMD1B, DDC, MAST4, MCF2L and PAX2.

Method 83. The method of any one of methods 71 to 81 wherein the at least one kidney differentially methylated region comprises a sequence having at least 90% identity to any one or more of SEQ ID NO. 1, SEQ ID NO. 2, SEQ ID NO. 8, SEQ ID NO. 9, SEQ ID NO. 15, SEQ ID NO. 16, SEQ ID NO. 22, SEQ ID NO. 23, SEQ ID NO. 29 or SEQ ID NO. 30.

Method 84. The method of any one of methods 71 to 83 wherein the method specifically detects damage to a defined tissue or cell-type of the kidney.

Method 84. The method of any one of methods 71 to 83 wherein the at least one kidney differentially methylated regions are located at at least one of MAST4 and DDC.

Method 85. The method of any one of methods 71 to method 83 wherein the at least one kidney differentially methylated regions are located at at least one of GRAMD1B and DDC.

Method 86. The method of any one of methods 71 to method 83 wherein the at least one kidney differentially methylated regions are located at at least one of GRAMD1B, DDC and PAX2.

Method 87. The method of any one of methods 71 to 86 wherein the subject is a human.

Method 88. The method of any one of methods 71 to 87 wherein the subject is non-human.

Method 89. The method of method 88 wherein the subject is a domesticated animal.

Method 90. The method of method 89 wherein the domesticated animal is a companion animal.

Method 91. The method of method 89 wherein the domesticated animal is selected from the group consisting of sheep, cattle, horses, cats, dogs, pigs, and chickens.

Method 92. The method of method 91 wherein the companion animal is selected from cats and dogs.

Method 93. At least one nucleotide primer sequence or nucleotide probe sequence when used in any one of methods 1 to 92 to detect at least one kidney differentially methylated region of cfDNA.

Method 94. The method of method 93 wherein the at least one nucleotide primer is two nucleotide primers when used in a PCR to detect at least one kidney differentially methylated region of cfDNA.

Use 1. Use of at least one kidney differentially methylated region in cfDNA in the manufacture of a reagent for diagnosing kidney damage in a subject.

Use 2. The use of use 1 wherein the reagent is at least one nucleotide primer or nucleotide probe, and in certain examples, two nucleotide primers configured to detect at least one kidney differentially methylated region in cfDNA.

### Examples

### Identification of Epigenetic Markers

DNA Methylation data from normal tissue and kidney cell types that was generated using Illumina's Infinium Human Methylation 450K or EPIC array was sourced from publicly available data including The Cancer Genome Atlas (TCGA) database and Gene Expression Omnibus (GEO) database. A total of 1,643 samples were downloaded, processed and collated. Cohort details are set out in Table 13 and Table 14.

TABLE 13
Kidney tissues and cell included in bioinformatic analysis.

| Kidney Tissues | Sample Number |
|---|---|
| Human Cultured podocytes | 2 |
| Human Renal Cortical Epithelial cells | 1 |
| Bulk Kidney | 140 |
| Renal Proximal Tubule Epithelial Cells | 31 |

TABLE 14
Other tissue sources included in bioinformatic analysis.

| Other Tissues | Sample Number |
|---|---|
| Amniotic membrane | 4 |
| Bile duct | 9 |
| Bladder | 21 |
| Breast | 97 |
| Cervix | 3 |
| Chorionic membrane | 4 |
| Colon | 37 |
| Complete hydatiform mole | 1 |
| Cord blood | 1 |
| Oesophagus | 16 |
| Granulocytes | 18 |
| Head and Neck | 50 |
| Left atrium | 11 |
| Liver | 50 |
| Lung | 74 |
| Pancreas | 10 |
| PBMC | 36 |
| Placental chorionic villi | 49 |
| Placental decidua | 3 |
| Placental mesenchyme | 3 |
| Placental trophoblast | 3 |
| Prostate | 50 |
| Rectum | 8 |
| Stomach | 2 |
| Thymus | 2 |
| Thyroid gland | 56 |
| Umbilical cord | 1 |
| Whole blood | 4 |
| Whole maternal blood | 662 |

DMRs were identified for hypermethylated regions using TCGABiolinks function TCGAanalyze_DMC. Mean methylation differences were determined between bulk kidney tissue and other bulk tissue sources. A Wilcoxon test using the Benjamini-Hochberg adjustment method was used to estimate p-values. A differential methylation difference of >0.25 with a false discovery rate (FDR)-adjusted Wilcoxon rank-sum P-value of <10-0.5 was used to identify hypermethylated probes. The threshold for differential methylation was chosen to enable detection of DMRs that were driven by high methylation levels within specific cell types.

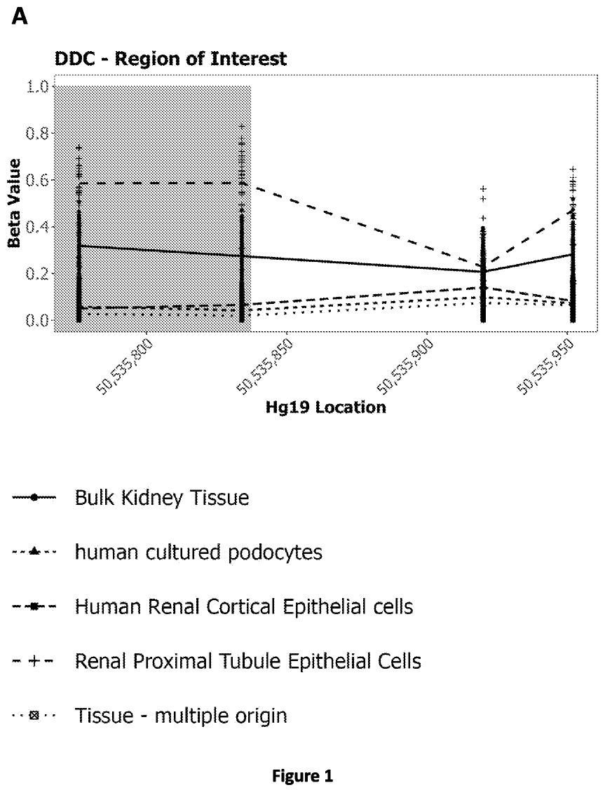

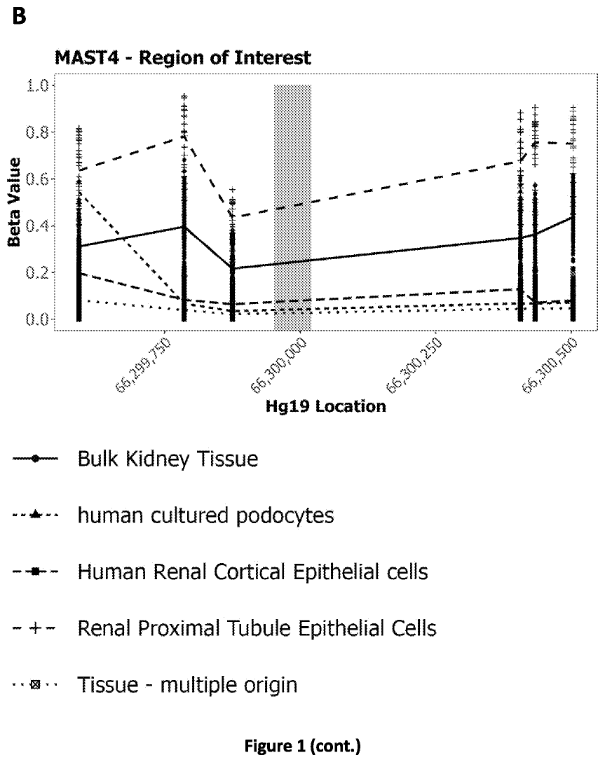

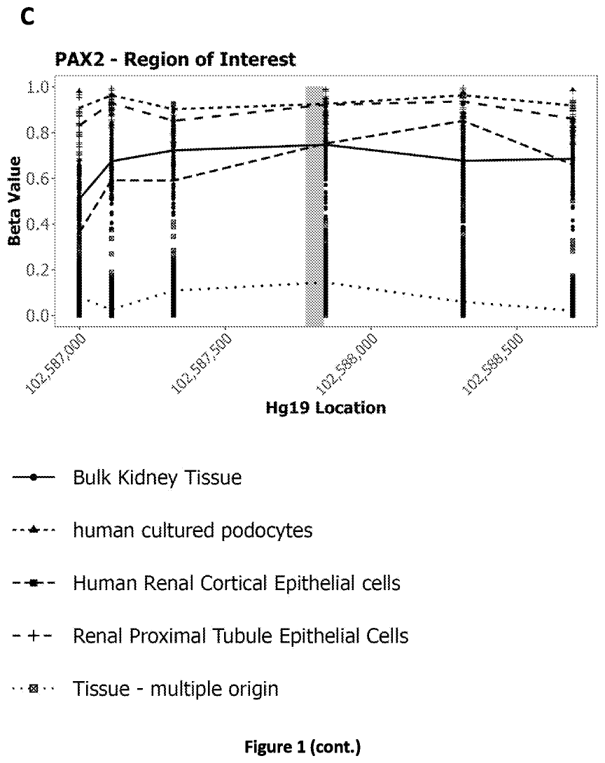

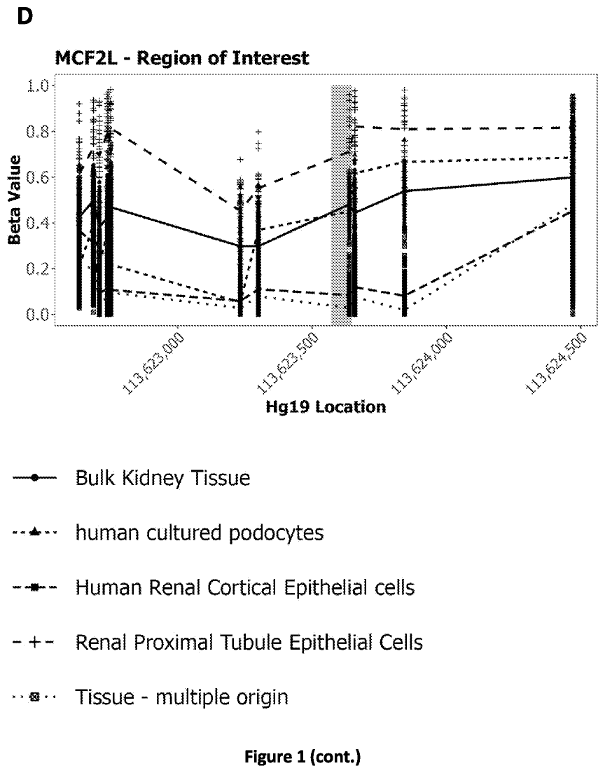

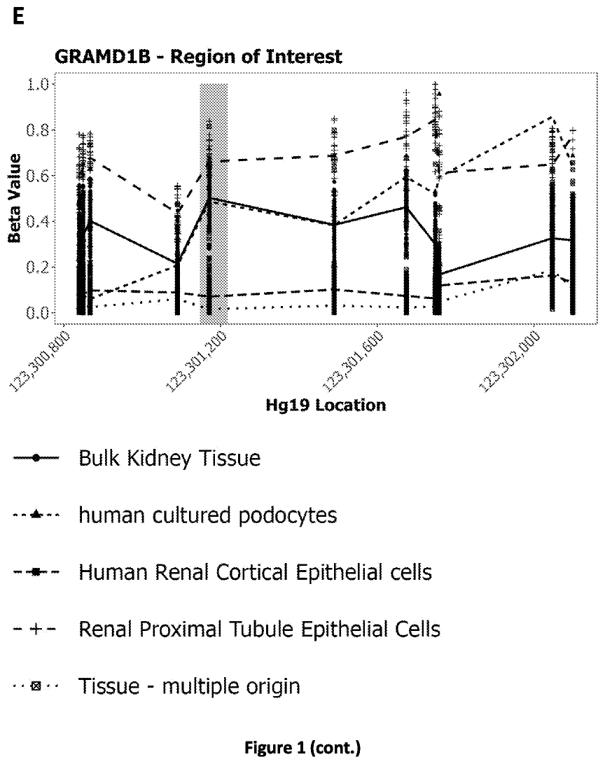

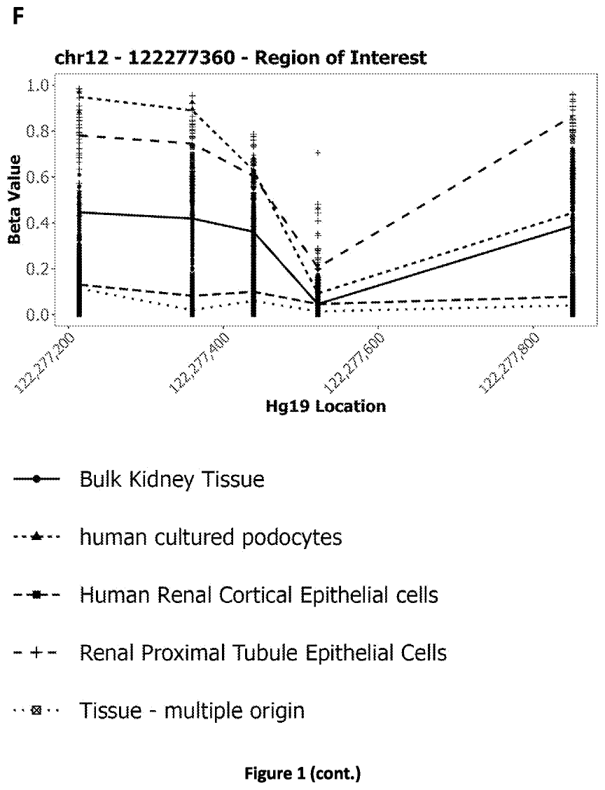

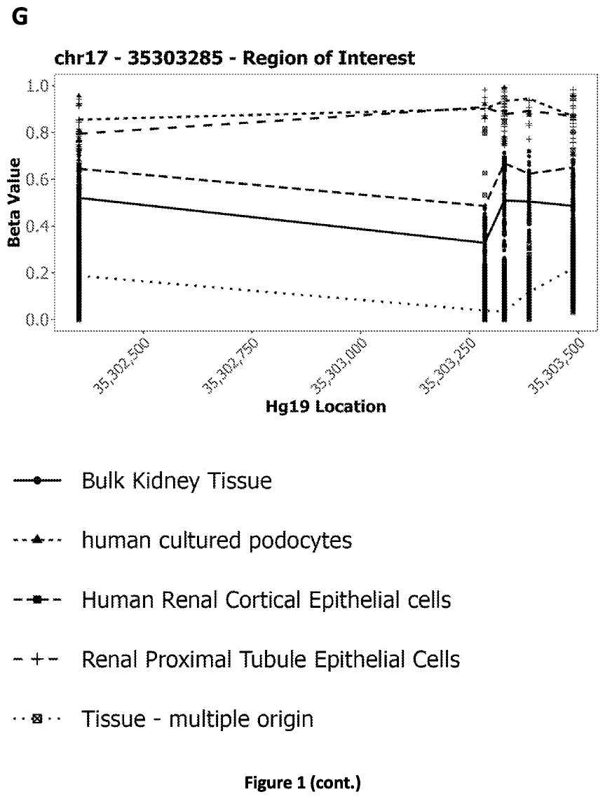

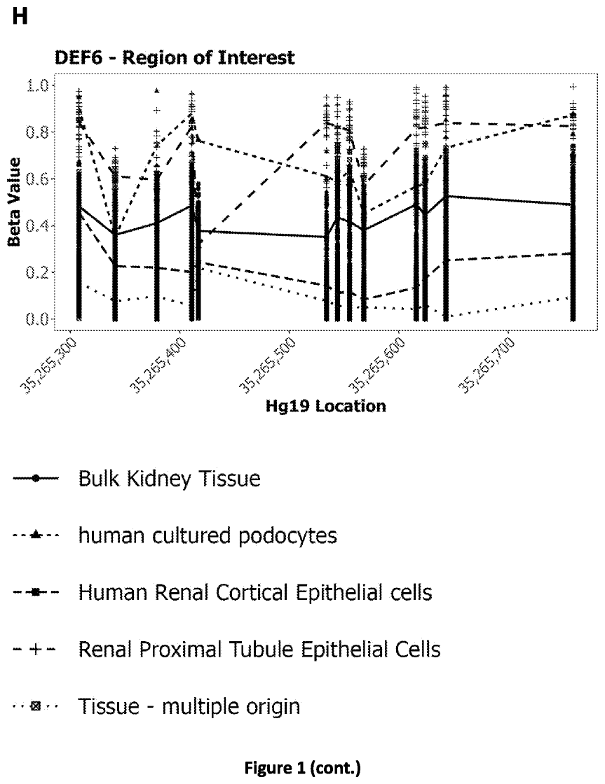

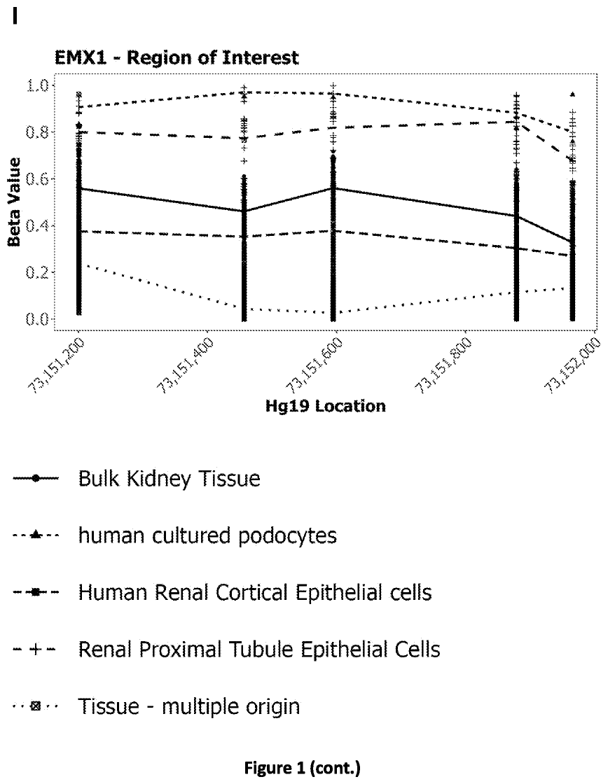

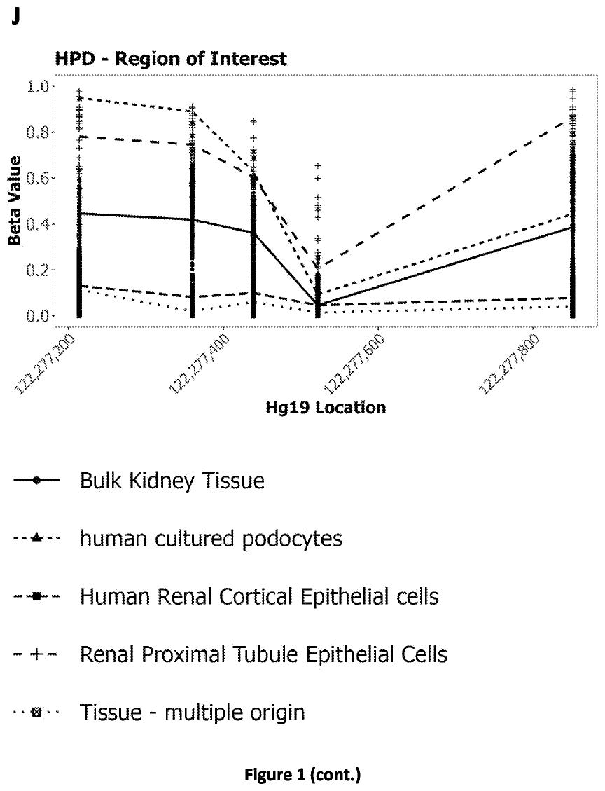

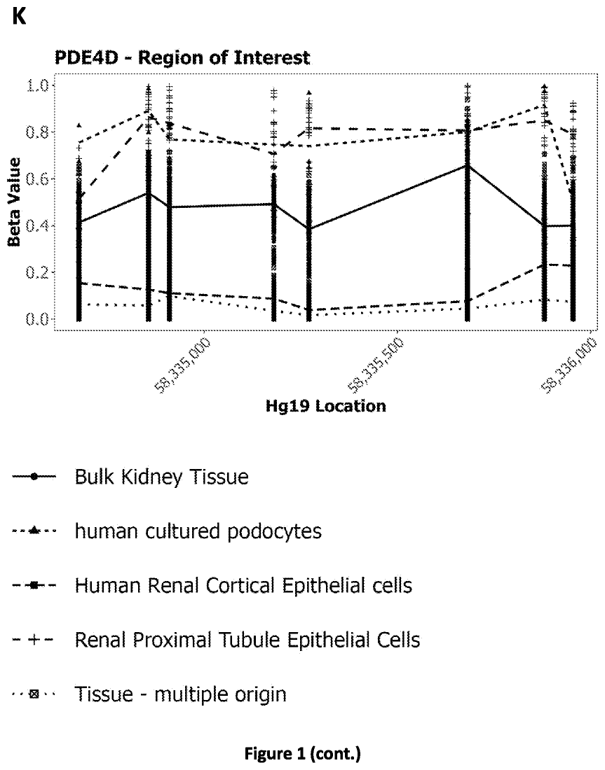

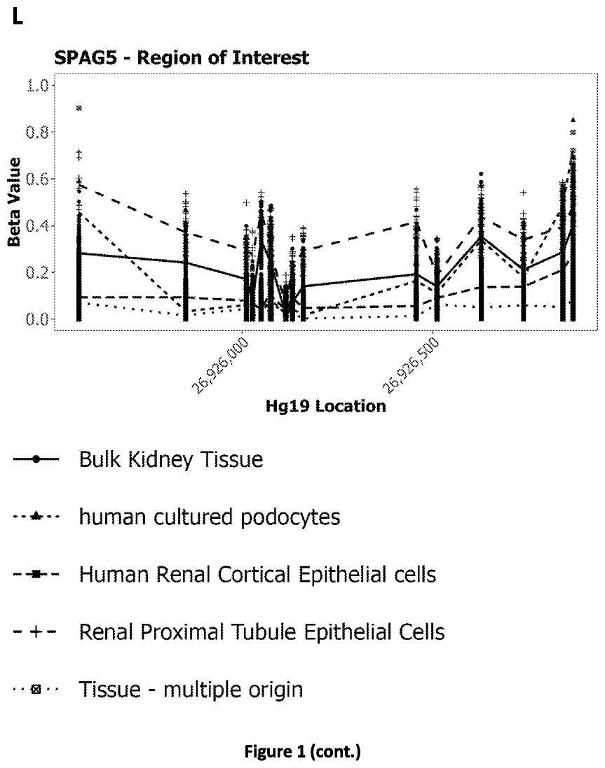

For candidate CpG probes, methylation levels were plotted across the region within 5,000 base pairs (FIG. 1). Different kidney cell types were included in these plots including Human Renal Proximal Tubule Epithelial Cells (RPTEC), Human Cultured Podocytes and Human Renal Cortical Epithelial cells. These plots were individually reviewed, and a shortlist was created (Table 15) based on separation between tissue types and large methylation differences between RPTEC and other tissues.

TABLE 15
Shortlisted regions from bioinformatic assessment.

| | Region of Interest |
|---|---|
| Assay | (Hg 19 location) |
| DDC | chr7: 50534754-50536754 |
| MAST4 | chr5: 66298953-66300953 |
| PAX2 | chr10: 102586777-102588835 |
| MCF2L | chr13: 113622573-113624573 |
| GRAMD1B | chr11: 123300149-123302149 |
| chr12 - 122277360 (CLIP1) | chr12: 122276360-122278360 |
| chr17 - 35303285 | chr17: 35302285-35304285 |
| DEF6 | chr6: 35264568-35266568 |
| EMX1 | chr2: 73150457-73152457 |
| HPD | chr12: 122276439-122278439 |
| PDE4D | chr5: 58334180-58336180 |
| SPAG5 | chr17: 26925050-26927050 |

From these shortlisted probes, five were used to design methylation-specific PCR assays; DDC, MAST4, PAX2, MCF2L and GRAMD1B.

### Detecting DNA Methylation Status

cfDNA was extracted from biological samples (plasma, urine, tissue etc) using a QIAamp circulating nucleic acid kit (Qiagen, Cat #55114) as per the manufacturer's instructions. The eluted DNA was then bisulfite converted using EZ DNA Methylation-Lightning kit (Zymo, Cat #D5030), or EpiTect Fast DNA Bisulfite kit (Qiagen, Cat #59824), as per the manufacturer's instructions. The resulting bisulfite converted DNA was analysed using qPCR or dPCR assays designed to amplify the target strands of one or more of the following targets: PAX2 (SEQ ID NOS 5-7), GRAMD1B (SEQ ID NOs 12-14), DDC (SEQ ID NOS 19-21), MAST4 (SEQ ID NOS 26-28) or MCF2L (SEQ ID NOS 33-35). ACTB amplification was also used as a control to ensure that the extraction (SEQ ID NOs 38-40 for detection of native ACTB sequence), or bisulfite conversion and PCR worked (SEQ ID NOs 43-45 for detection of bisulfite converted ACTB sequence). qPCR reactions comprised 7.5 μL GoTaq Hot Start Colourless mastermix, 2 mM MgCl₂, 200 nM of each Forward and Reverse primers, 100 nM fluorescently labelled hydrolysis probe, and template DNA made up to 15 μL final volume, and were cycled as follows: 95° C., 2 mins; [95° C., 15 secs; 62° C., 30 secs, 72° C., 30 secs with acquisition]×50; 40° C., 10 secs, on a QuantStudio7 real-time PCR system (ThermoFisher). For digital PCR, reactions comprised 10 μL QIAcuity probe PCR mix (Qiagen, Cat #250103), 800 nM of each Forward and Reverse primers, 400 nM fluorescently labelled hydrolysis probe, and template DNA made up to 40 μL final volume, and were cycled as follows: 95° C., 2 mins; [95° C., 15 secs; 61° C., 30 secs with acquisition]×40 on the QIAcuity 4 plate digital PCR system (Qiagen).

The following sections set out the sequences of the DMRs and the primers and probes that were used to determine methylation status in these DMRs (based on hg19).

### Human Chr10 (+): 102,505,468—102,590,402—Paired Box 2 (PAX2) Gene; NM 000278.5

Chr10 (+): 102,586,126-102,588,109-CpG Island in which the PAX2 amplicon target resides.

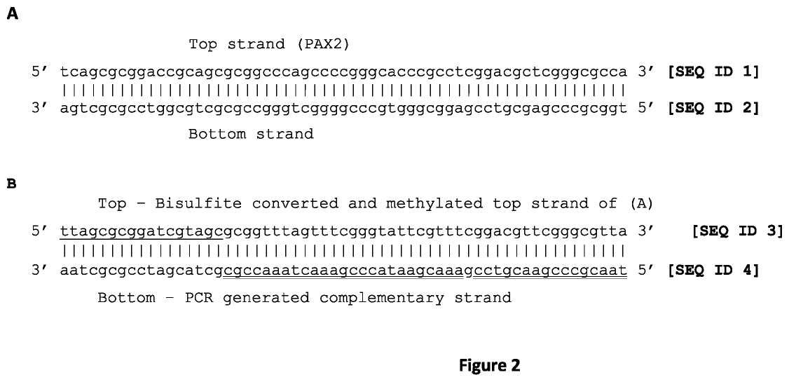

Chr10 (+): 102,587,777-102,587,835-Native sequence of PAX2 PCR assay region (59 bp). The sequence of the top strand (SEQ ID NO. 1) and the bottom strand (SEQ ID NO. 2) of the PCR assay region are shown in FIG. 2A.

The sequence of the bisulfite-converted methylated top strand (SEQ ID NO. 3) and the PCR-generated complementary strand (SEQ ID NO. 4) are shown in FIG. 2B. The forward and reverse primers, and the oligonucleotide probe used to detect the bisulfite-converted methylated DNA are listed in Table 16.

TABLE 16

| SEQ | | |
|---|---|---|
| ID | | |
| NO. | Sequence | Description |
| 5 | ttagcgcggatcgtag | PAX2 forward primerDetecting the bisulfite |
| | c | and methylated region residing Chr10 (+): |
| | | 102,587,777-102,587,793. |
| 6 | taacgcccgaacgtcc | PAX2 reverse primerDetecting the bisulfite |
| | | and methylated region residing Chr10 (−): |
| | | 102,587,820-102,587,835. |
| 7 | aaacgaatacccgaaa | PAX2 oligonucleotide probeDetecting the |
| | ctaaaccgc | bisulfite and methylated region residing |
| | | Chr10 (−): 102,587,794-102,587,818. |

### Human Chr11 (+): 123.229.130-123,498.475—GRAM Domain Containing 1B (GRAMD1B) Gene; NM 001367420

Chr11 (+): 123,301,050-123,302,149-CpG Island in which the GRAMD1B amplicon target resides.

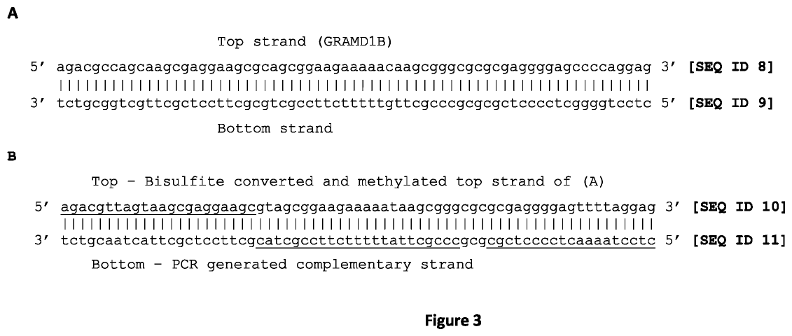

Chr11 (+): 123,301,149-123,301,216-Native sequence of GRAMD1B PCR assay region (67 bp). The sequence of the top strand (SEQ ID NO. 8) and the bottom strand (SEQ ID NO. 9) of the PCR assay region are shown in FIG. 3A.

The sequence of the bisulfite-converted methylated top strand (SEQ ID NO. 10) and the PCR-generated complementary strand (SEQ ID NO. 11) are shown in FIG. 3B. The forward and reverse primers, and the oligonucleotide probe used to detect the bisulfite-converted methylated DNA are listed in Table 17.

TABLE 17

| SEQ | | |
|---|---|---|
| ID | | |
| NO. | Sequence | Description |
| 12 | agacgttagtaagcgagga | GRAMD1B forward primerDetecting the bisulfite |
| | agc | and methylated region residing Chr11 (+): |
| | | 123,301,149-123,301,170. |
| 13 | ctcctaaaactcccctcgc | GRAMD1B reverse primerDetecting the bisulfite |
| | | and methylated region residing Chr11 (−): |
| | | 123,301,198-123,301,216. |
| 14 | cccgcttatttttcttccg | GRAMD1B oligonucleotide probeDetecting the |
| | ctac | bisulfite and methylated region residing |
| | | Chr11 (−): 123,301,171-123,301,194. |

Human Chr7 (+): 50,526, 140-50,633,102—Dopa Decarboxylase (DDC) Gene; NM 001082971.2 Chr7 (+): 50,535,741-50,535,953-CpG Island in which the DDC amplicon resides.

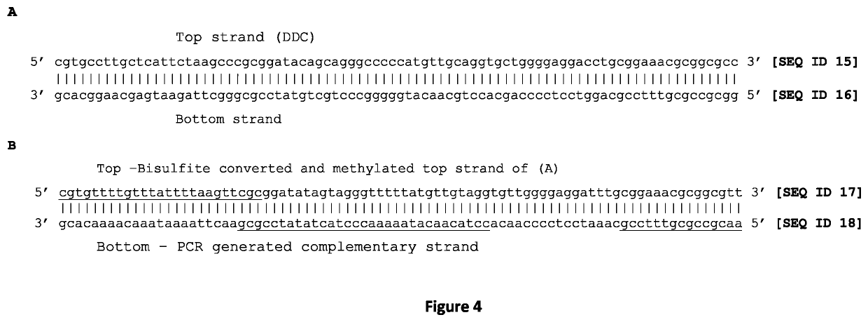

Chr7 (+): 50,535,754-50,535,837-Native sequence of DDC PCR assay region (84 bp). The sequence of the top strand (SEQ ID NO. 15) and the bottom strand (SEQ ID NO. 16) of the PCR assay region are shown in FIG. 4A.

The sequence of the bisulfite-converted methylated top strand (SEQ ID NO. 17) and the PCR-generated complementary strand (SEQ ID NO. 18) are shown in FIG. 4B. The forward and reverse primers, and the oligonucleotide probe used to detect the bisulfite-converted methylated DNA are listed in Table 18.

TABLE 18

| SEQ | | |
|---|---|---|
| ID | | |
| NO. | Sequence | Description |
| 19 | cgtgttttgtt | DDC forward primerDetecting the |
| | tattttaagtt | bisulfite and methylated region |
| | cgc | residing Chr7 (+): |
| | | 50,535,754-50,535,778. |
| 20 | aacgccgcgtt | DDC reverse primerDetecting the |
| | tccg | bisulfite and methylated region |
| | | residing Chr7 (−): |
| | | 50,535,823-50,535,837. |
| 21 | cctacaacata | DDC oligonucleotide probe |
| | aaaaccctact | Detecting the bisulfite and |
| | atatccgcg | methylated region residing Chr7 |
| | | (−): 50,535,776-50,535,806. |

### Human Chr5 (+): 65,892,221-66,465,421—Microtubule Associated Serine/Threonine Kinase Family Member 4 (MAST4) Gene; NM 001164664.2

Chr5 (+): 66,299,769-66,300,083-CpG Island in which the MAST4 amplicon target resides.

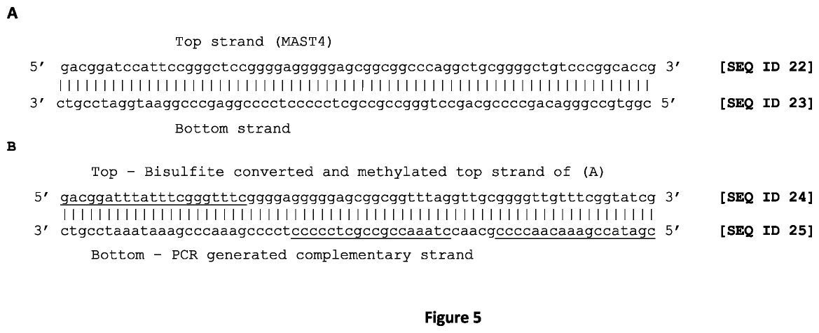

Chr5 (+): 66,299,953-66,300,019-Native sequence of MAST4 PCR assay region (67 bp). The sequence of the top strand (SEQ ID NO. 22) and the bottom strand (SEQ ID NO. 23) of the PCR assay region are shown in FIG. 5A.

The sequence of the bisulfite-converted methylated top strand (SEQ ID NO. 24) and the PCR-generated complementary strand (SEQ ID NO. 25) are shown in FIG. 5B. The forward and reverse primers, and the oligonucleotide probe used to detect the bisulfite-converted methylated DNA are listed in Table 19.

TABLE 19

| SEQ | | |
|---|---|---|
| ID | | |
| NO. | Sequence | Description |
| 26 | gacggatttatttcgggt | MAST4 forward primerDetecting the bisulfite |
| | ttc | and methylated region residing Chr11 (+): |
| | | 66,299,953-66,299,973. |
| 27 | cgataccgaaacaacccc | MAST4 reverse primerDetecting the bisulfite |
| | | and methylated region residing Chr11 (−): |
| | | 66,300,002-66,300,019. |
| 28 | ctaaaccgccgctccccc | MAST4 oligonucleotide probeDetecting the |
| | | bisulfite and methylated region residing |
| | | Chr11 (−): 66,299,979-66,299,996. |

### Human Chr13 (+): 113,623,528-113,754,056—MCF.2 Cell Line Derived Transforming Sequence-Like (MCF2L) Gene; NM 001112732.3

Chr13 (+): 113,622,738-113,623,660-CpG Island in which the MCF2L amplicon target resides.

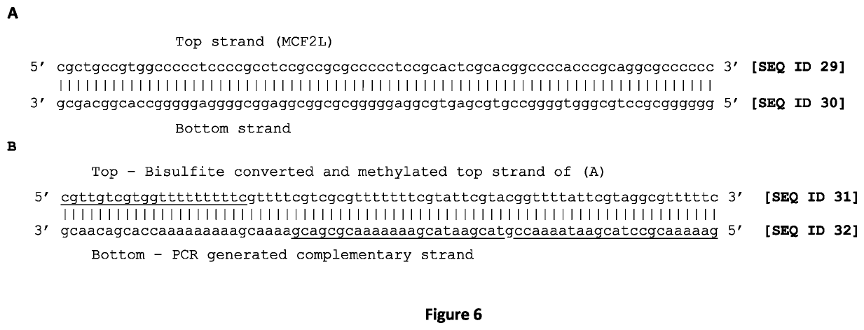

Chr13 (+): 113,623,573-113,623,646-Native sequence of MCF2L PCR assay region (74 bp). The sequence of the top strand (SEQ ID NO. 29) and the bottom strand (SEQ ID NO. 30) of the PCR assay region are shown in FIG. 6A.

The sequence of the bisulfite-converted methylated top strand (SEQ ID NO. 31) and the PCR-generated complementary strand (SEQ ID NO. 32) are shown in FIG. 6B. The forward and reverse primers, and the oligonucleotide probe used to detect the bisulfite-converted methylated DNA are listed in Table 20.

TABLE 20

| SEQ | | |
|---|---|---|
| ID | | |
| NO. | Sequence | Description |
| 33 | cgttgtcgtggtttttttttc | MCF2L forward primerDetecting the bisulfite |
| | | and methylated region residing Chr13 (+): |
| | | 113,623,573-113,623,593. |
| 34 | gaaaaacgcctacgaataaaa | MCF2L reverse primerDetecting the bisulfite |
| | cc | and methylated region residing Chr13 (−): |
| | | 113,623,624-113,623,646. |
| 35 | tacgaatacgaaaaaaacgcg | MCF2L oligonucleotide probeDetecting the |
| | acg | bisulfite and methylated region residing |
| | | Chr13 (−): 113,623,599-113,623,622. |

### Human Chr7 (−): 5.566,779-5.570.232 (+2 kb Upstream Region=>5,566.779-5,572,232)—ACTB Gene; NM 001101

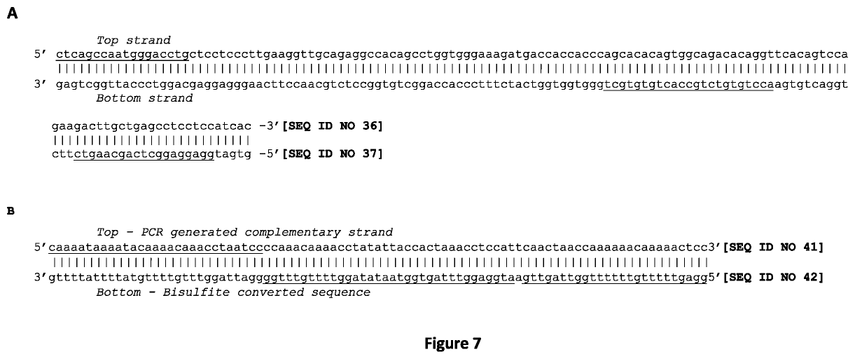

Chr7 (−): 5,571,726-5,571,859-target region of a PCR assay used to quantify the yield of total genomic DNA (134 bp). The amplicon is situated in the +2 kb upstream promoter region. The sequence of the top strand (SEQ ID NO. 36) and the bottom strand (SEQ ID NO. 37) of the PCR assay region are shown in FIG. 7A. The forward and reverse primers, and the oligonucleotide probe used to detect the ACTB sequence are listed in Table 21.

TABLE 21

| SEQ | | |
|---|---|---|
| ID | | |
| NO. | Sequence | Description |
| 38 | ggaggaggctcagcaagt | ACTB forward primerDetecting the genomic region |
| | c | residing Chr7 (+): 5,571,836-5,571,854. |
| 39 | ctcagccaatgggacctg | ACTB reverse primerDetecting the genomic region |
| | | residing Chr7 (−): 5,571,726-5,571,743. |
| 40 | acctgtgtctgccactgt | ACTB oligonucleotide probeDetecting the genomic |
| | gtgct | region residing Chr7 (+): 5,571,800-5,571,822. |

Bisulfite converted sequence of the bottom strand of the genomic region located at Chr7 (+): 5,572,176-5,572,265 (89 bp). The sequence of the PCR generated top strand (SEQ ID NO. 41) and the bisulfite-converted bottom strand (SEQ ID NO. 42) are shown in FIG. 7B. The forward and reverse primers, and the oligonucleotide probe used to detect the bisulfite-converted methylated DNA are listed in Table 22.

TABLE 22

| SEQ | | |
|---|---|---|
| ID | | |
| NO. | Sequence | Description |
| 43 | ggagtttttgttttttggttagttg | ACTB bisulfite forward primer. |
| | | Detecting the bisulfite and |
| | | methylated region residing Chr7 |
| | | (+): 5,572,241-5,572,265 |
| 44 | caaaataaaatacaaaacaaaccta | ACTB bisulfite reverse primer. |
| | atcc | Detecting the bisulfite and |
| | | methylated region residing Chr7 |
| | | (−): 5,572,176-5,572,204. |
| 45 | atggaggtttagtggtaatataggt | ACTB bisulfite oligonucleotide |
| | tttgtttgg | probeDetecting the bisulfite and |
| | | methylated region residing Chr7 |
| | | (+): 5,572,206-5,572,239. |

### Tissue Specificity

Loci that are differentially methylated in kidney cells/tissues relative to blood and other tissues were identified bioinformatically and specific primers and probes were designed to these regions as described above. A number of different assays were designed to each of five DMRs, and these were tested analytically to identify the best performing assays for each DMR (SEQ ID NOS 1-35).

In order to confirm the specificity of these assays, DNA from 14 different tissue types comprising adipose, adrenal, brain, breast, colon, heart, kidney, liver, lung, pancreas, skeletal, skin and spleen, alongside commercially sourced fully unmethylated DNA (CpGenome universal unmethylated DNA, Sigma, Cat #S7822) and Human genomic DNA from buffy coat (PBMC, Sigma, Cat #11691112001) were bisulfite converted using EZ DNA Methylation-Lightning kit (Zymo, Cat #D5030), or EpiTect Fast DNA Bisulfite kit (Qiagen, Cat #59824), as per the manufacturer's instructions. 5 ng each of bisulfite converted DNA from each tissue type, bisulfite converted unmethylated DNA, bisulfite converted PBMC DNA, and native PBMC DNA were amplified in quadruplicate by qPCR using primers and probes specific for PAX2 (SEQ ID NOS 5-7), GRAMD1B (SEQ ID NOS 12-14), DDC (SEQ ID NOS 19-21), MAST4 (SEQ ID NOS 26-28) or MCF2L (SEQ ID NOS 33-35). ACTB amplification was also used as a control to quantify native total DNA (SEQ ID NOS 38-40), or bisulfite converted total DNA (SEQ ID NOS 43-45). A standard curve for each assay was prepared by amplifying 2.5-fold serial dilutions of bisulfite converted fully methylated DNA (Zymo, Cat #D5011) from 5000 pg/reaction to 8.2 pg/reaction, or 500 copies/reaction to 0.82 copies/reaction, in quadruplicate and this was used to calculate the amount of DNA amplified for each gene. qPCR reactions comprised 7.5 μL GoTaq Hot Start Colourless mastermix, 2 mM MgCl₂, 200 nM of each Forward and Reverse primers, 100 nM fluorescently labelled hydrolysis probe, and template DNA made up to 15 μL final volume, and were cycled as follows: 95° C., 2 mins; [95° C., 15 secs; 62° C., 30 secs, 72° C., 30 secs with acquisition]×50; 40° C., 10 secs, on a QuantStudio7 real-time PCR system (ThermoFisher).

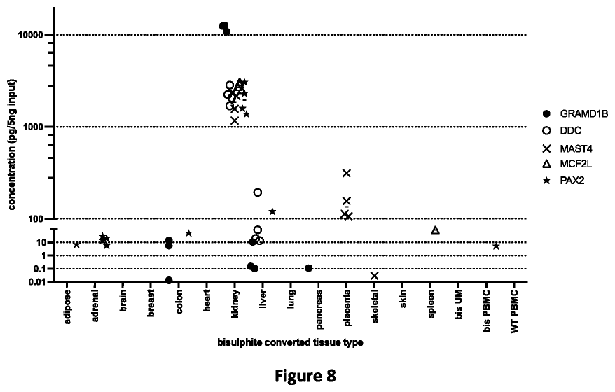

FIG. 8 shows the results of a representative assay designed against each of the five DMR genes assayed. It can be seen that each assay strongly detects kidney DNA as well as some low-level positivity in some other tissues. Typically, this off-target amplification is <1% of the target amplification, with an occasional sample being higher than this, but when applied to a cfDNA scenario, where typical yields are ˜10 ng/ml plasma or 5 ng/ml urine for total cfDNA, and only a fraction of this is derived from these organs/tissues, except liver, then this low-level positivity is generally inconsequential.

Sensitivity of Differentially Methylated Assays in Plasma from Presumed Healthy Donors

The five selected assays were then tested in plasma obtained from presumed healthy individuals under 30 years of age to determine if there was any background signal. cfDNA from two aliquots of 3 mL plasma was extracted using a QIAamp circulating nucleic acid kit (Qiagen, Cat #55114) as per the manufacturer's instructions. The eluted DNA from the two aliquots for each sample was combined and then bisulfite converted using EZ DNA Methylation-Lightning kit (Zymo, Cat #D5030), or EpiTect Fast DNA Bisulfite kit (Qiagen, Cat #59824), as per the manufacturer's instructions. An amount of bisulfite converted DNA equivalent to 1 mL plasma was analysed over 3 replicates in qPCR for each assay targeting PAX2 (SEQ ID NOS 5-7), GRAMD1B (SEQ ID NOS 12-14), DDC (SEQ ID NOS 19-21), MAST4 (SEQ ID NOS 26-28) or MCF2L (SEQ ID NOS 33-35). ACTB (SEQ ID NOS 43-45) was also amplified as a control to demonstrate that the extraction, bisulfite conversion and PCR worked and also to determine total yield of amplifiable DNA, independent of methylation status. A standard curve for each assay was prepared by amplifying 2.5-fold serial dilutions of bisulfite converted fully methylated DNA (Zymo, Cat #D5011) from 5000 pg/reaction to 8.2 pg/reaction, or 500 copies/reaction to 0.82 copies/reaction, in quadruplicate and this was used to calculate the amount of DNA amplified for each gene. qPCR reactions comprised 7.5 μL GoTaq Hot Start Colourless mastermix, 2 mM MgCl₂, 200 nM of each Forward and Reverse primers, 100 nM fluorescently labelled hydrolysis probe, and template DNA made up to 15 μL final volume, and were cycled as follows: 95° C., 2 mins; [95° C., 15 secs; 62° C., 30 secs, 72° C., 30 secs with acquisition]×50; 40° C., 10 secs, on a QuantStudio7 real-time PCR system (ThermoFisher).

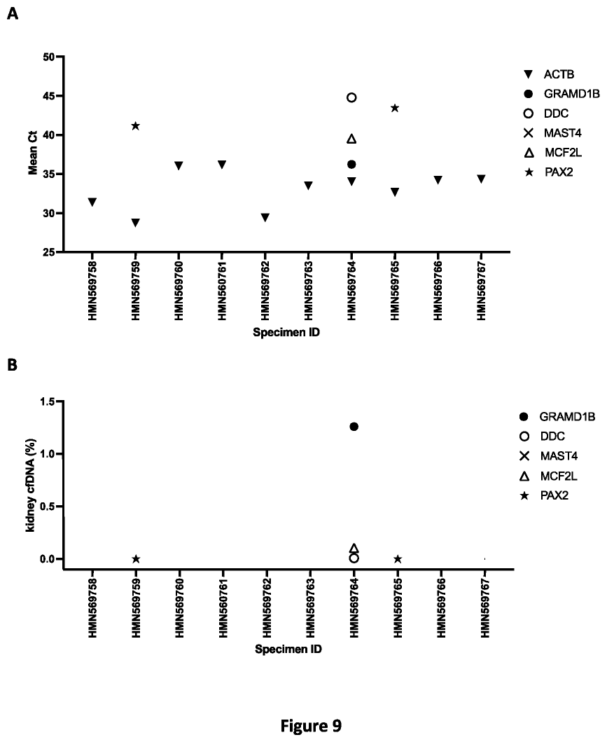

FIG. 9 shows that very little signal is obtained for any of the kidney-specific assays in plasma and where a signal was detected, the % of the total kidney cfDNA was typically ≤0.1%. Only GRAMD1B gave a higher signal than this (1.26% of total kidney cfDNA) in one sample only. As these samples are from presumed healthy donors, rather than from individuals who are confirmed to be free of kidney disease, it is possible that donor HMN569764 had an underlying kidney condition that gave rise to the low-level positivity seen in 3 of the 5 markers tested.

Sensitivity of Differentially Methylated Assays in Urine from Presumed Healthy Donors

The five selected assays were then tested in urine obtained from 20 presumed healthy individuals aged 26-61 to determine if there was any background signal. The cfDNA from two aliquots of 3 mL urine was extracted using a QIAamp circulating nucleic acid kit (Qiagen, Cat #55114) as per the manufacturer's instructions. The eluted DNA from the two aliquots for each sample was combined and then bisulfite converted using EZ DNA Methylation-Lightning kit (Zymo, Cat #D5030), or EpiTect Fast DNA Bisulfite kit (Qiagen, Cat #59824), as per the manufacturer's instructions. An amount of bisulfite converted DNA equivalent to 1 mL urine was analysed in dPCR for each assay targeting PAX2 (SEQ ID NOS 5-7), GRAMD1B (SEQ ID NOS 12-14), DDC (SEQ ID NOS 19-21), MAST4 (SEQ ID NOS 26-28), or MCF2L (SEQ ID NOS 33-35). ACTB (SEQ ID NOs 43-45) was also amplified as a control to demonstrate that the extraction, bisulfite conversion and PCR worked and also to determine total yield of amplifiable DNA, independent of methylation status. dPCR reactions comprised 10 μL QIAcuity probe PCR mix (Qiagen, Cat #250103), 800 nM of each Forward and Reverse primers, 400 nM fluorescently labelled hydrolysis probe, and template DNA made up to 40 μL final volume, and were cycled as follows: 95° C., 2 mins; [95° C., 15 secs; 61° C., 30 secs with acquisition]×40 on the QIAcuity 4 plate digital PCR system (Qiagen).

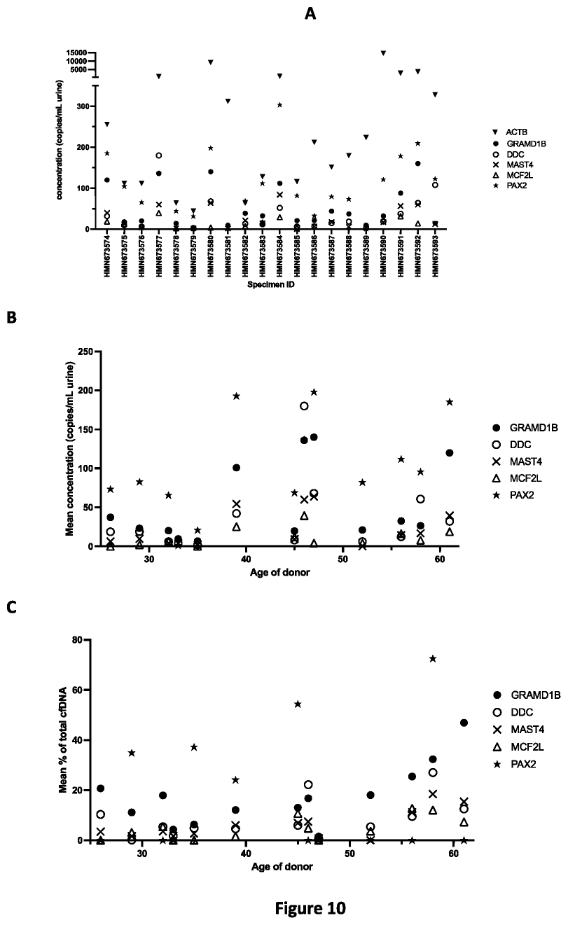

FIG. 10 shows that the situation is quite different in urine, compared to plasma. All of the differentially methylated loci amplify strongly and contribute a significant amount of signal (mostly ˜1-20%) compared to total kidney cfDNA. As these samples were obtained from patients with a range of ages, we looked to see if there was a correlation with age and increasing signal, and it was noticed that this was indeed the case, especially when considering the signal as a percentage of total cfDNA. An increase in amount or proportion of kidney-specific cfDNA may be indicative of acute kidney injury or chronic kidney disease and could indicate a decline in kidney function with age.

### Sensitivity of Kidney-Specific PCR at Low DNA Input

The sensitivity of each of the five kidney-specific assays was evaluated in contrived samples containing very low concentrations of fully methylated, bisulfite converted DNA (Zymo, Cat #D5011). The fully methylated DNA was bisulfite converted using EZ DNA Methylation-Lightning kit (Zymo, Cat #D5030), or EpiTect Fast DNA Bisulfite kit (Qiagen, Cat #59824), as per the manufacturer's instructions. The eluted DNA was quantified in dPCR using the ACTB assay (SEQ ID NOs 43-45) and subsequently diluted in 1 ng/ml cRNA as a stabiliser so that each PCR well contained 0.17 copies of amplifiable DNA. A 384-well qPCR plate was set up for each kidney-specific assay containing either 320 or 343 wells of low concentration bisulfite converted methylated DNA and a standard curve for each assay was prepared by amplifying 2.5-fold serial dilutions of bisulfite converted fully methylated DNA (Zymo, Cat #D5011) from 5000 pg/reaction to 8.2 pg/reaction, or 500 copies/reaction to 0.82 copies/reaction, in quadruplicate. qPCR reactions for each assay targeting PAX2 (SEQ ID NOS 5-7), GRAMD1B (SEQ ID NOS 12-14), DDC (SEQ ID NOS 19-21), MAST4 (SEQ ID NOS 26-28), or MCF2L (SEQ ID NOS 33-35) comprised 7.5 μL GoTaq Hot Start Colourless mastermix, 2 mM MgCl₂, 200 nM of each Forward and Reverse primers, 100 nM fluorescently labelled hydrolysis probe, and template DNA made up to 15 μL final volume, and were cycled as follows: 95° C., 2 mins; [95° C., 15 secs; 62° C., 30 secs, 72° C., 30 secs with acquisition]×50; 40° C., 10 secs, on a QuantStudio7 real-time PCR system (ThermoFisher). Three consecutive wells were combined to emulate a sample tested in triplicate. Therefore, individual replicate positivity is expected at 17% and sample positivity at ˜50% if assays are performing optimally. Table 23 shows that all these assays are performing near perfect.

TABLE 23
Summary of individual replicate positivity, sample positivity (3 replicates
combined) and 95% confidence intervals for % positivity of the proportion
for a variety of kidney-specific assays directed against the differentially
methylated regions of GRAMD1B, DDC, MAST4, MCF2L and PAX2 genes. Each
well contained 0.17 copies of DNA, as measured by dPCR.

| | Replicate positivity | | | Sample positivity | | |
|---|---|---|---|---|---|---|
| | No Positive/ | % | | No Positive/ | % | |
| Assay | total No wells | Positivity | 95% CI | total No wells | Positivity | 95% CI |
| GRAMD1B | 55/320 | 17.2 | 13.2-21.7 | 49/107 | 45.8 | 36.1-55.7 |
| DDC | 50/320 | 15.6 | 11.8-20.1 | 41/107 | 38.3 | 29.1-48.2 |
| MAST4 | 52/320 | 16.3 | 12.3-20.7 | 42/107 | 39.3 | 30.0-49.2 |
| MCF2L | 45/320 | 14.1 | 10.4-18.3 | 38/107 | 35.5 | 26.5-45.3 |
| PAX2 | 72/343 | 21.0 | 16.8-25.7 | 61/114 | 53.5 | 43.9-62.9 |

Detection of Kidney-Specific cfDNA in Kidney Transplant Patients

DNA was extracted from 57 clinical specimens taken from 25 kidney transplant patients at various time points pre- and post-renal transplant. Where available, the DNA from the equivalent of 1 mL plasma for each time point was extracted using a QIAamp circulating nucleic acid kit (Qiagen, Cat #55114) as per the manufacturer's instructions. The eluted DNA was then bisulfite converted using EZ DNA Methylation-Lightning kit (Zymo, Cat #D5030), or EpiTect Fast DNA Bisulfite kit (Qiagen, Cat #59824), as per the manufacturer's instructions. The resulting bisulfite converted DNA was analysed using qPCR or dPCR assays designed to amplify the target strands of the following targets: PAX2 (SEQ ID NOS 5-7), GRAMD1B (SEQ ID NOS 12-14), and DDC (SEQ ID NOS 19-21). ACTB amplification was also used as a control to ensure that the extraction, bisulfite conversion and PCR worked (SEQ ID NOS 43-45). qPCR reactions comprised 7.5 μL GoTaq Hot Start Colourless mastermix, 2 mM MgCl₂, 200 nM of each Forward and Reverse primers, 100 nM fluorescently labelled hydrolysis probe, and template DNA made up to 15 μL final volume, and were cycled as follows: 95° C., 2 mins; [95° C., 15 secs; 62° C., 30 secs, 72° C., 30 secs with acquisition]×50; 40° C., 10 secs, on a QuantStudio7 real-time PCR system (ThermoFisher). For digital PCR, reactions comprised 10 μL QIAcuity probe PCR mix (Qiagen, Cat #250103), 800 nM of each Forward and Reverse primers, 400 nM fluorescently labelled hydrolysis probe, and template DNA made up to 40 μL final volume, and were cycled as follows: 95° C., 2 mins; [95° C., 15 secs; 61° C., 30 secs with acquisition]×40 on the QIAcuity 4 plate digital PCR system (Qiagen).

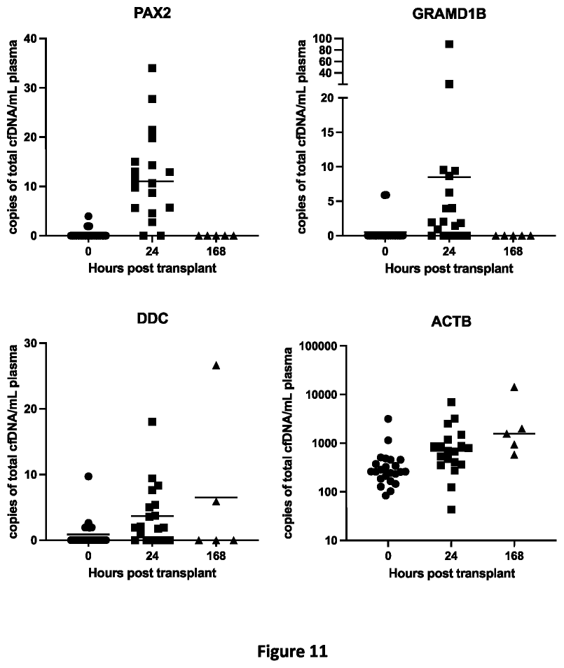

The number of copies per mL of plasma per sample at each time point was examined and results show that there is considerably more total cfDNA DNA present in samples taken post-transplant (mean=1190c ACTB/mL at 24 h and 3849 c/mL at 168h post-transplant) compared to 437.5 c/mL ACTB pre-transplant (FIG. 11). This difference is more marked when comparing the kidney-specific cfDNA concentrations. PAX2 has mean concentration of 0.34 c/mL pre-transplant, and 12.08 c/mL at 24 h and 0c/mL at 168h; GRAMD1B has mean concentration of 0.51 c/mL pre-transplant, 8.47 c/mL at 24 h and 0c/mL at 168h; and DDC has mean concentration of 0.88 c/mL pre-transplant, and 3.67 c/mL at 24 h and 6.51c/mL at 168h. With regard to the low levels of kidney-specific cfDNA detectable pre-transplant, these patients are generally in renal failure and have no functional kidney remaining prior to transplant. The levels of cfDNA detected are typically highest within 24 hours post-transplant and start to return to baseline levels by day 7. These results are consistent with results using donor-derived cfDNA to monitor transplant. PAX2 provides the highest level of signal, with 1-1.2% of the total cfDNA within the 24 hours post-transplant.

Identifying Specificity of Biomarkers from Different Kidney Cell Types

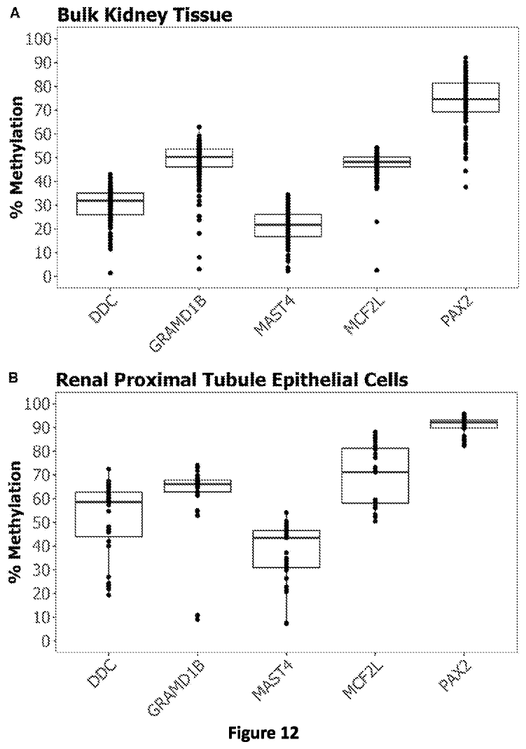

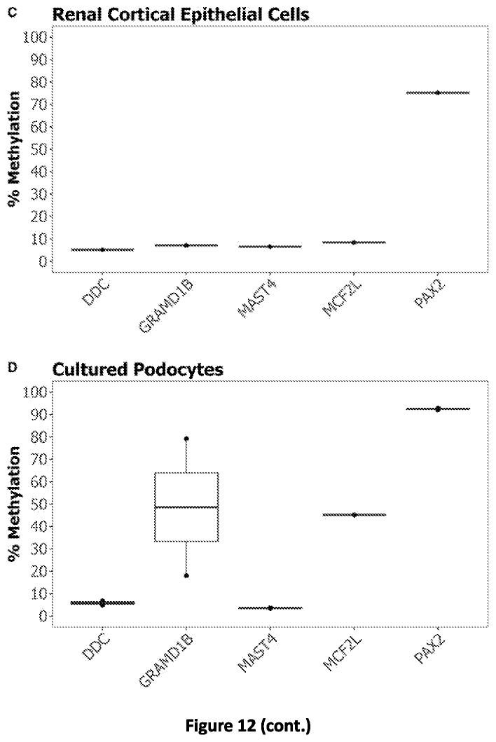

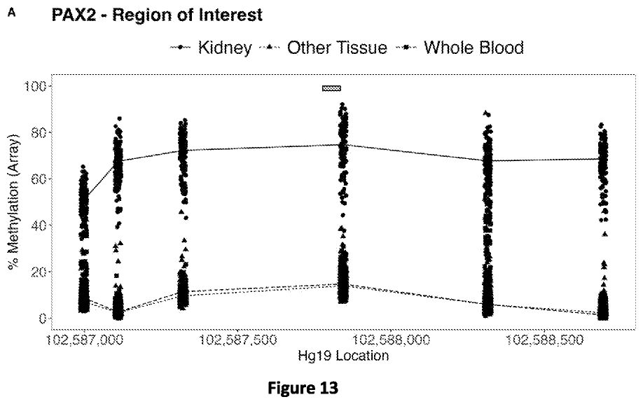

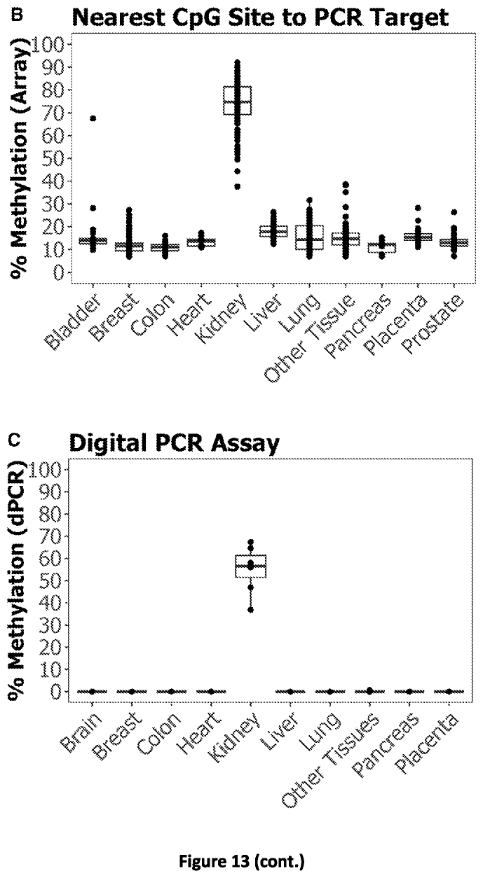

Cell specific Biomarkers: Cell-specific data is less abundant in the public domain. To identify differentially methylated regions in cells within the kidney, a 0.25 methylation difference cut off was used to identify low-level differentially methylation regions in bulk kidney tissue and then be further evaluated in cell-specific data to determine the methylation proportion in these specific cell types. Data was sourced for Renal Proximal Tubule Epithelial Cells (31 samples from human subjects GSE115227, GSE145745, and GSE126441), human cultured podocytes (2 samples from human subject GSE41689), and human Renal Cortical Epithelial cells (1 sample from human subject GSE126441). These cell types were selected to evaluate the methylation status of the identified probes that exhibited low levels of methylation as shown in FIG. 12.

As shown in FIG. 12, PAX2 (cg23206032) was found to be highly methylated in all cell types and was the only region developed for PCR that was positive in the renal cortical epithelial cells. Both DDC and MAST4 were specific for the renal proximal tubule epithelial cells (RPTEC). This could be advantageous for early detection of injury and prognosis given the crucial role of RPTEC's in kidney function, including reabsorption of water, electrolytes, and nutrients. MCF2L and GRAMD1B had a similar profile to DDC and MAST4 but did show methylation in podocytes.

### PCR Assay Development

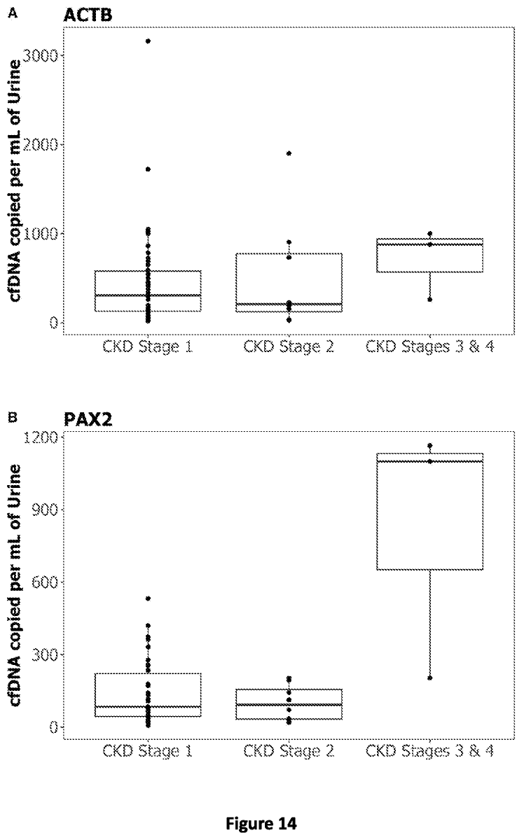

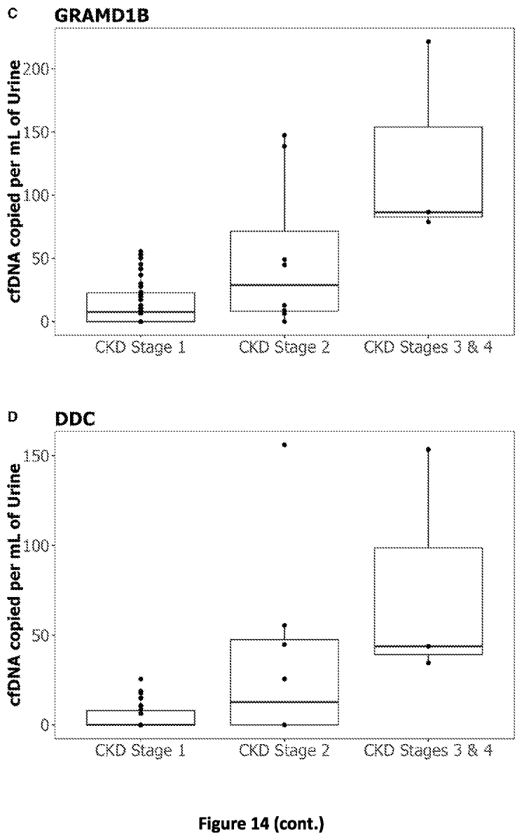

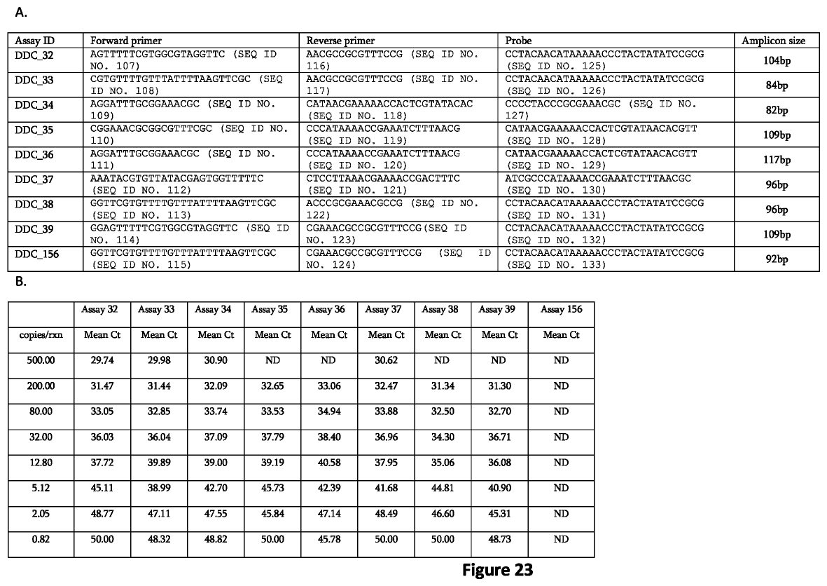

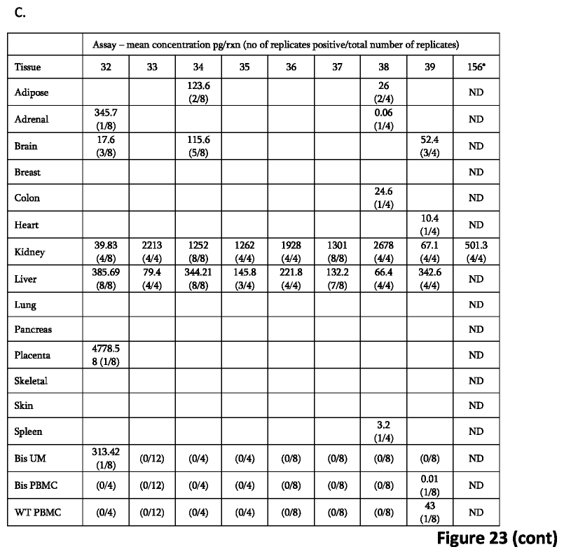

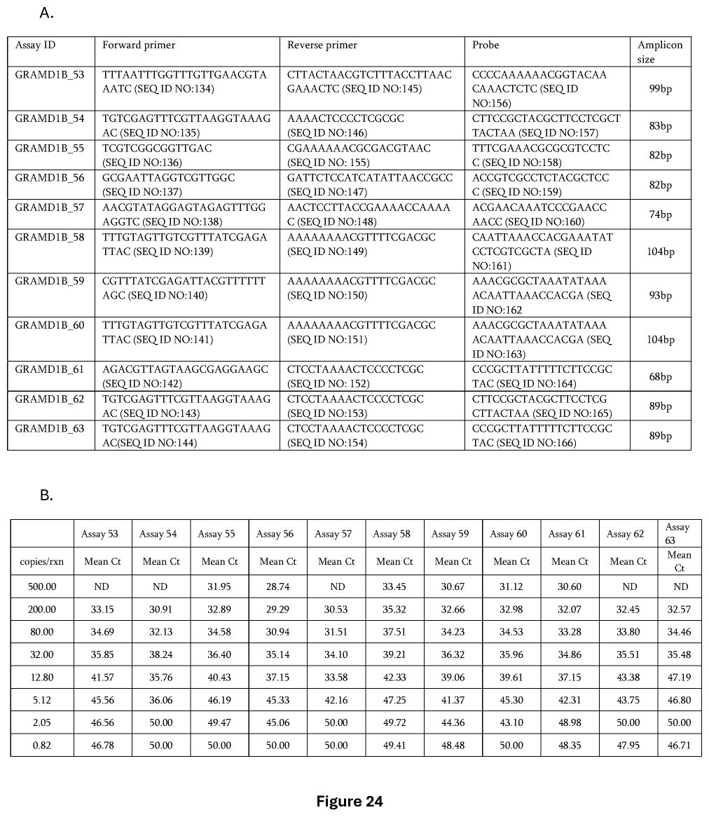

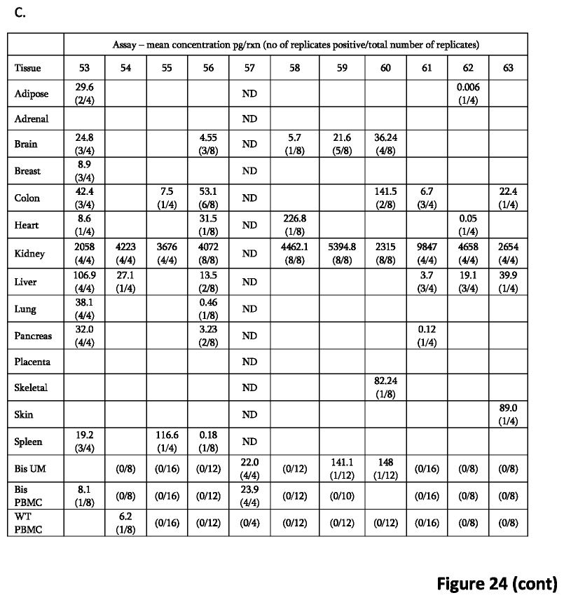

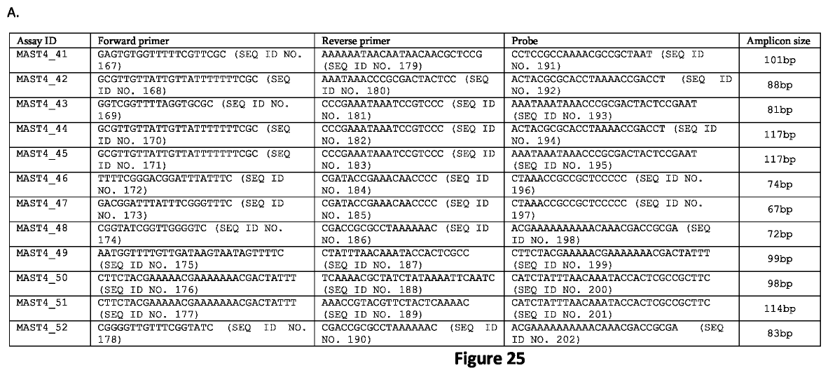

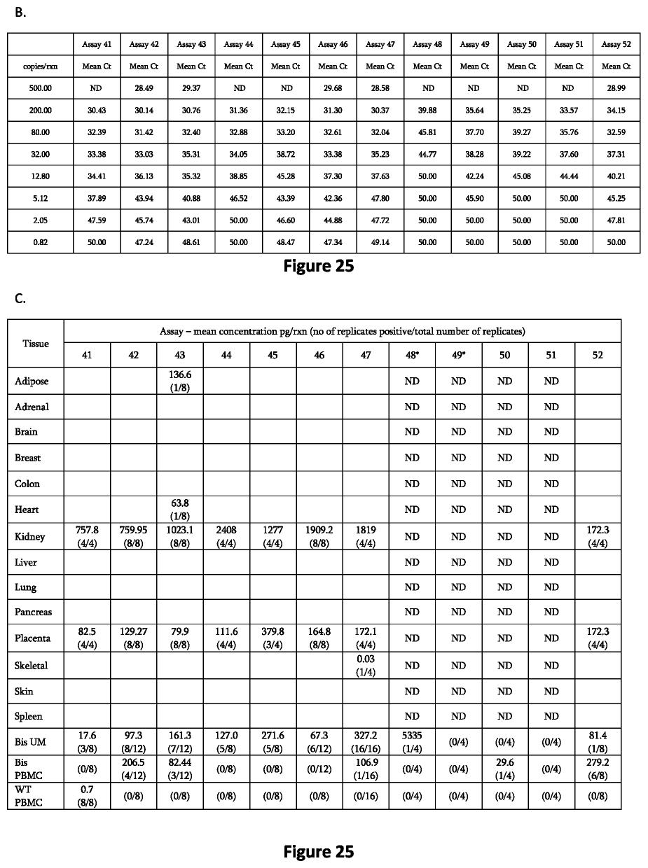

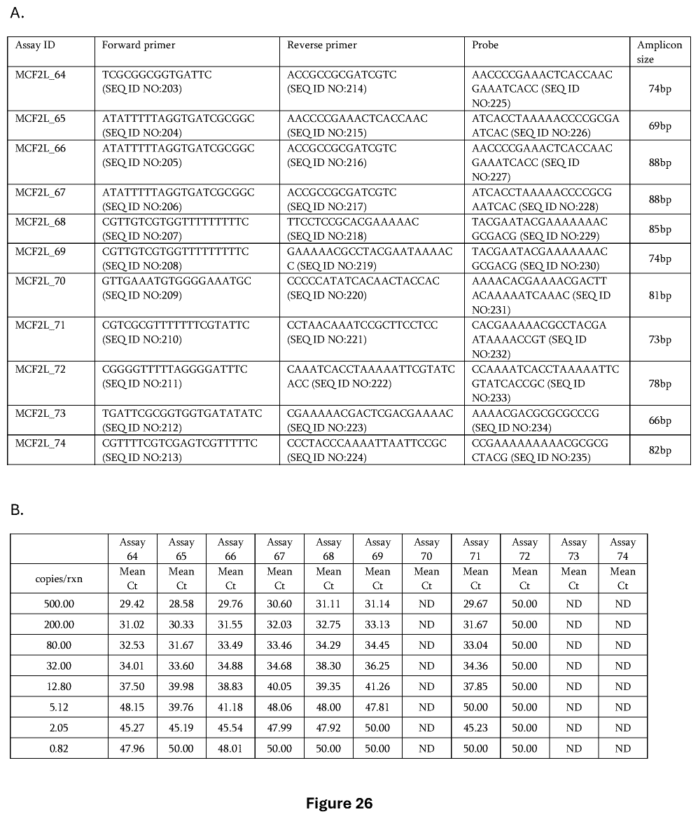

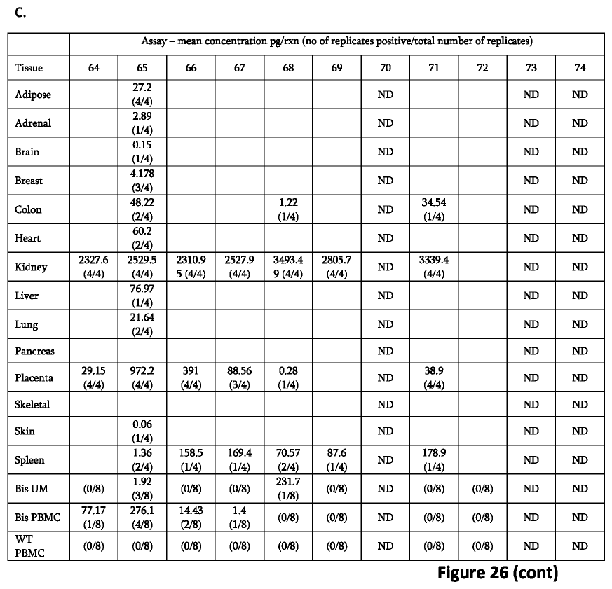

A number of methylation specific PCR primers and probes were designed for all 5 regions, namely GRAMD1B, DDC, MAST4, MCF2L, PAX2 (Panel A, FIGS. 23-27). Each assay was assessed with a 7-point 2.5-fold serial dilution of bisulphite converted methylated DNA (Zymo D5011) from 500 to 0.82 cps/rxn with ≥4 replicates. A Ct of 50 indicates that the amplification failed (Panel B, FIGS. 23-27). Specificity was assessed using bisulphite converted unmethylated DNA (placental DNA that is hypomethylated; bis UM)) and bisulphite converted (bis PBMC) or native peripheral blood mononuclear cell DNA (WT PBMC) (Panel C, FIGS. 23-27). Assay designs with amplification down to at least 12.8 cps/rxn, with no or minimal detection of bisulphite converted unmethylated DNA or PBMC DNA, were selected for further assessment. Next, assay designs were tested in a panel of 15 normal tissues (Panel C, FIGS. 23-27). DNA sourced from these tissue types was added at 5 ng/rxn and assessed via digital PCR in at least 4 replicates. ND indicates not done, and a blank well indicates no amplification in any replicates. As shown in FIG. 14, profiles observed were similar to array data with exceptional specificity for DNA extracted from bulk kidney tissue. A triplex assay was prepared for PAX2, DDC and GRAMD1B. The preferred PCR designs to detect human kidney differentially methylated regions have been included in FIGS. 23-27 and Tables 12, 16-20.

Kidney Specific Methylation Biomarker Comparison in Urine Samples from Subjects with Various Stages of CKD.

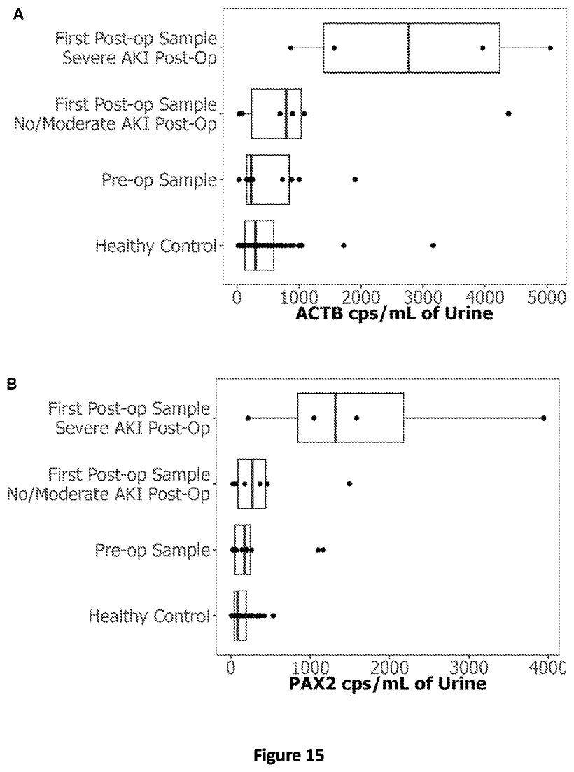

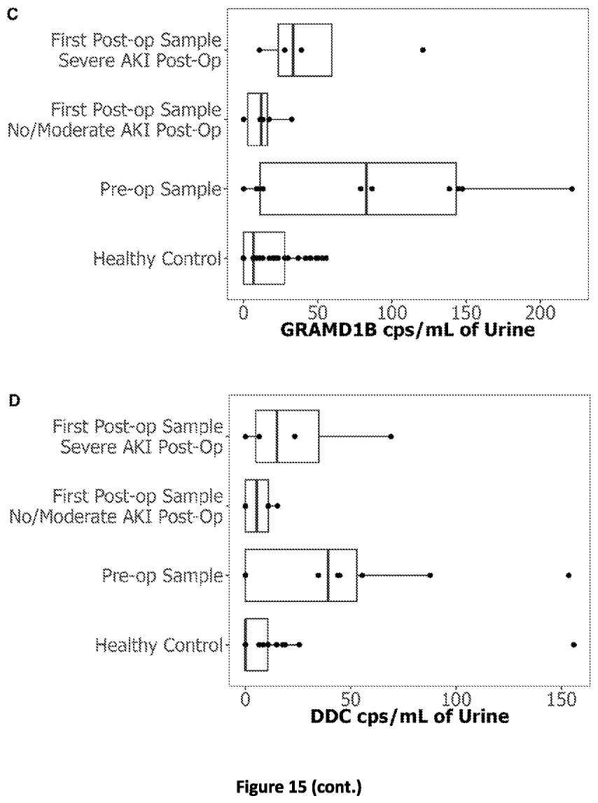

Kidney specific methylation biomarkers PAX2, GRAMD1B and DDC were detected in urine samples using the above described dPCR methodology (FIG. 15) PAX2, GRAMD1B and DDC all show statistically relevant differences (p value=0.0409, 0.0020 and 0.0033, respectively). Patients were categorised based on eGFR determined using CKD-EPI 2021 equation. The results from PAX2 are noticeably different to GRAMD1B and DDC results, where the latter are a better representation of the Renal Proximal Tubule Epithelial Cells (RPTECs) and provide the potential for earlier detection of CKD and prediction of renal function decline. Notably, lower levels of DNA were detected for DDC and GRAMD1B relative to PAX2.

Kidney Specific Methylation Biomarker Comparison in Urine Samples from Healthy Patients and Subjects Pre- and Post-Heart Transplant Surgery

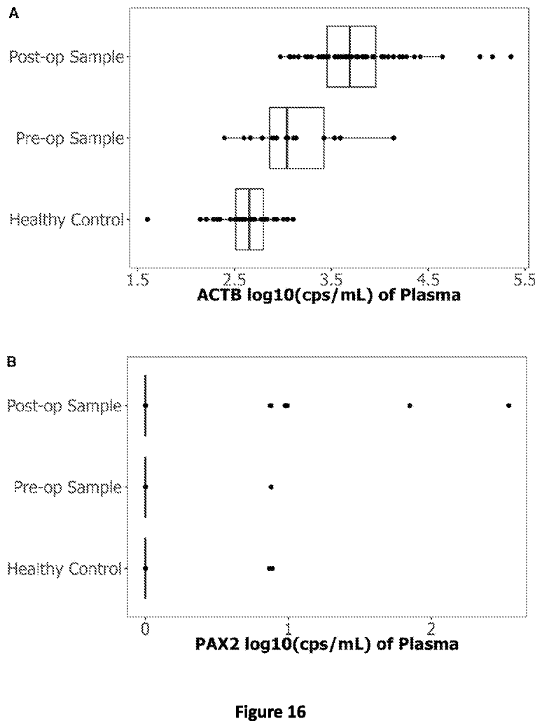

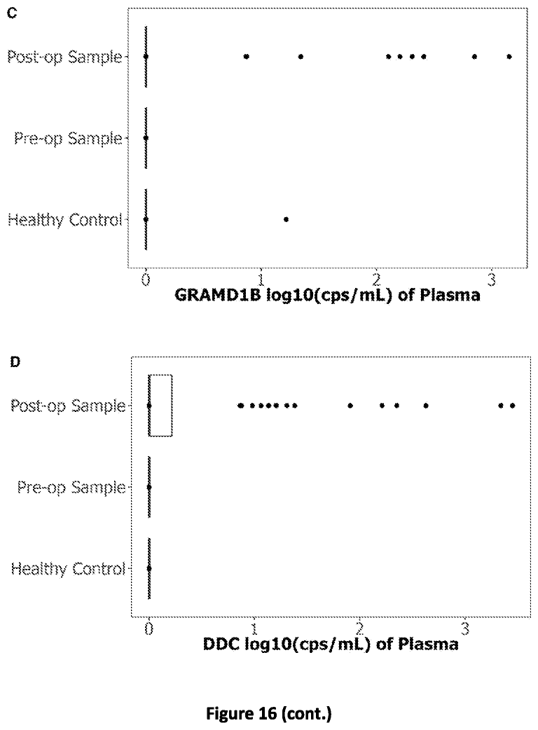

Urine samples were collected from presumed healthy adult cohort with no known kidney disease and patients pre- and post-heart transplant surgery. Post-transplant samples were split into two groups, those with severe AKI within 48 hours post-surgery and those with no or moderate AKI post-surgery. Categorisation was determined based on RIFLE criteria for fold change in creatine from baseline. ACTB is a measurement total cfDNA and shows increased levels in heart transplant patients with severe AKI post-surgery (see FIG. 16). Thus, kidney cfDNA release as a result of injury increased total cfDNA in urine. However, the level of total cfDNA measurement lacks specificity and does not discriminate between cfDNA increases due to other biological variability including exercise, inflammation, and infection.

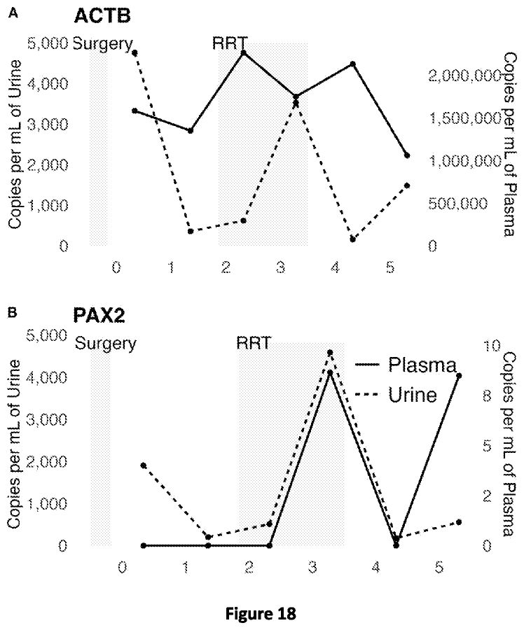

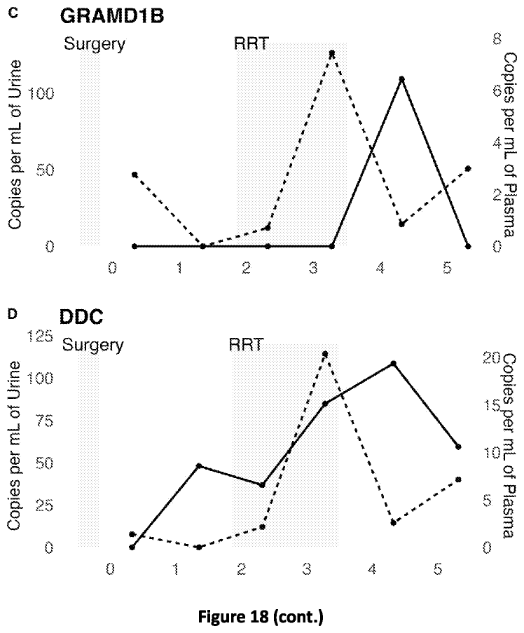

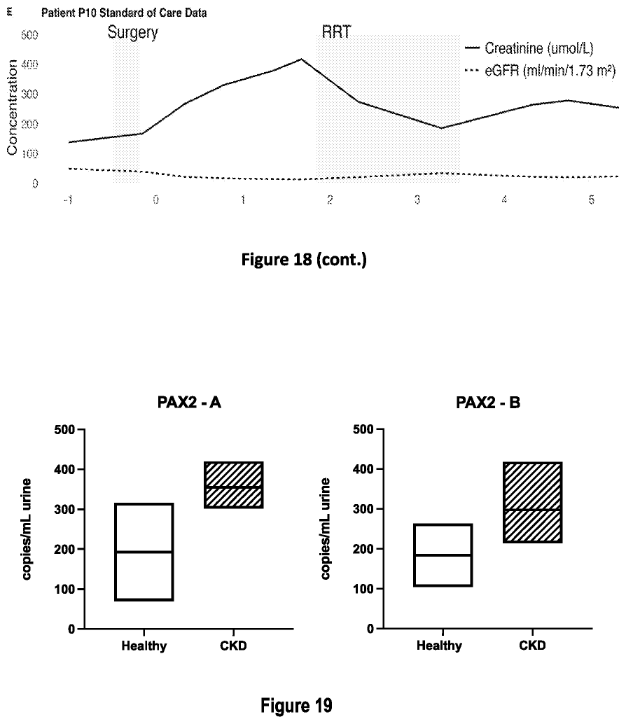

There is a marked increase PAX2 in the first sample post-surgery and this is evident prior to detectable changes in creatinine (refer to FIGS. 18 and 19). The tubular specific markers (GRAMD1B and DDC) provide a different profile where increase in marker concentration in the sample post-surgery (within 24 hours) is modest but there is a large variability in pre-op samples. These markers are also elevated in patients with CKD consistent with FIG. 15. This suggests that GRAMD1B and DDC markers provide insight into kidney frailty with the potential to be predictive for patient outcomes such as post-surgical AKI or CKD related renal decline.

Kidney Specific Methylation Biomarker Comparison in Plasma from Healthy Patients and Subjects Pre- and Post-Heart Transplant Surgery

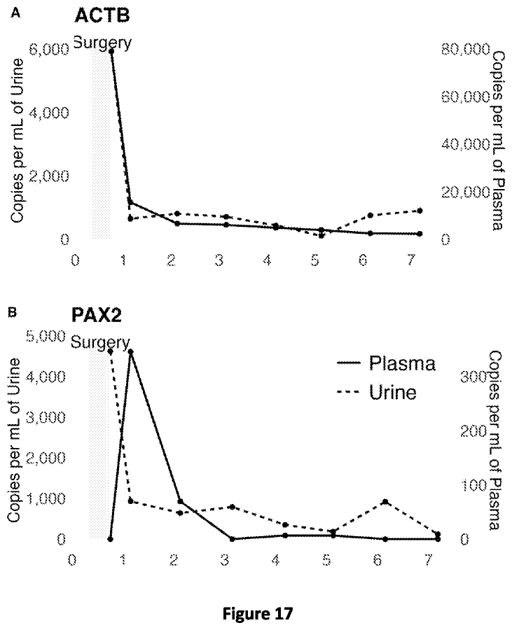

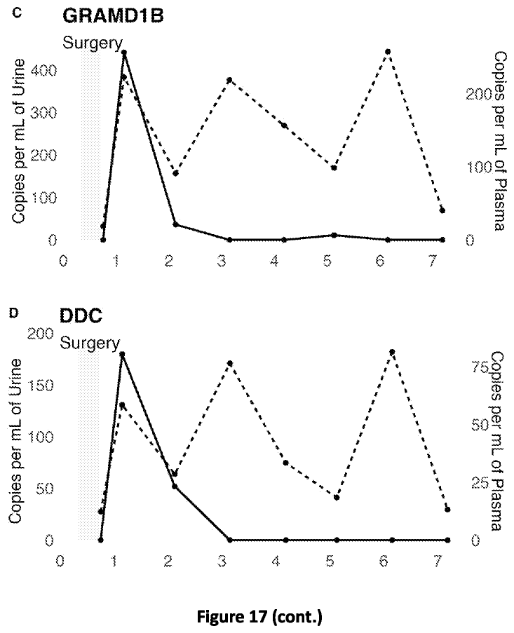

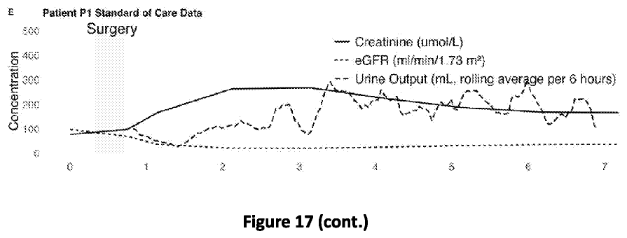

As shown in FIG. 17, very few of the healthy cohort and pre surgery samples had detectable kidney DNA in plasma. Post heart transplant 3 out of 10 patients had detectable plasma kidney differentially methylated cfDNA at multiple timepoints, refer to FIGS. 18 and 19 for examples. Only 1 mL of plasma was used in these examples and increasing sample input volume while further optimising the assay performance is likely to increase sensitivity. Furthermore, the discordance between plasma and urine suggests the potential utility of plasma in identifying diverse injury and disease types through the release of kidney cfDNA from various pathophysiologies. This has promising implications for improving patient selection and stratification in therapeutic applications.

Serial Hourly Testing of Methylation Biomarker Comparison in Urine from Patient 1 and Patient 10 Post Heart Transplant

Biomarker values were generated using dPCR as described above. FIG. 18A-D show total cfDNA (A) and kidney specific cfDNA (B-D) in both urine (left y-axis) and plasma (right-axis). Figure E provides the standard of care markers for the patient, including creatinine (μmol/mL), eGFR (ml/min/1.73 m²) and urine output (mL) using a rolling 6-hour average based on hourly readings. The time of x-axis for all plots is time from surgery. As shown in Figure E, the patient had a steep rise in creatinine with a greater than 3-fold increase in creatinine over baseline post-surgery, concomittant with a decrease in urine output. Urine output returned to normal levels within 24 hours, but a high creatinine persisted until 9 days post-surgery (data not shown). Total cfDNA (FIG. 18A) increases dramatically in both urine and plasma (from 815 cps/mL and 813 cps/mL at baseline to 5,928 cps/mL and 225,884 cps/mL in urine and plasma, respectively) but drops sharply within 24 hours and offers little insight into damage within the kidney. The bulk kidney cfDNA marker, PAX2, is elevated in urine immediately post-surgery (5,928 cps/mL of urine) over baseline (226 cps/mL of urine) 10 hours prior to creatinine increasing to 2.14 over baseline and 33 hours prior to exceeding a 3-fold increase. The corresponding matched blood sample for PAX2 is low for the first sample but highly elevated for the second sample, where urine PAX2 cfDNA is decreasing. This indicates that urine is the primary sample for early detection and release of kidney cfDNA in plasma is delayed. It also indicates that although total cfDNA increases dramatically in plasma post-surgery, this does not contain kidney cfDNA and thus total cfDNA is a poor marker for assessment of kidney damage. The tubular specific (DDC) and tubular/podocyte specific (GRAMD1B) markers in cfDNA are low until the second sample (˜24 hours post surgery) and may indicate the delay in release of tubular DNA post injury and represent acute tubular necrosis. From this sample the tubular specific cfDNA concentration remains elevated and erratic and can be a predictor of long-term kidney damage. The levels seen in Patient 1 have only been repeated in patients receiving dialysis in ICU. Prior to surgery this patient was classed as stage 2 CKD (eGFR=79.1 ml/min/1.73 m²) however had an elevated kidney cfDNA in a pre-surgical urine sample (226, 154, 50 cps/mL for PAX2, GRAMD1B and DDC, respectively).

FIG. 19. The same assay conditions described for Patient 1 were also performed for Patient 10 (although there is no urine output data for Patient 10 shown in FIG. 19) Patient 10 underwent hemodiafiltration while in ICU. Patient 10 also exhibited an increase in PAX2 post-surgery (1,904 cps/mL of urine) compared with pre-op sample (1,245 cps/mL of urine). During hemodiafiltration when the urine output is expected to be low there is a sharp increase in kidney specific cfDNA. This has been observed in all patients that received hemodiafiltration while in ICU and is independent of urine output volume. This indicates that during hemodiafiltration kidney cell death is occurring and it is also resulting in tubular cell death. Without the cell-specific markers, it would not be possible to determine if this damage was occurring within the tubular cells but clearly the damage is clinically meaningful.

Identifying kidney damage occurring within the RPTECs is clinically significant for several reasons. RPTECs play a crucial role in kidney function, including reabsorption of essential substances and maintenance of electrolyte balance. Damage to RPTECs can disrupt these critical functions, leading to impaired kidney function and potentially contributing to the development or progression of kidney diseases.

By determining if the observed damage is specifically occurring within the RPTECs, clinicians can gain insights into the underlying mechanisms and pathophysiology of the kidney injury. This knowledge can help guide treatment strategies and interventions targeted towards preserving RPTEC function and promoting renal recovery. Identifying RPTEC damage also provides important diagnostic and prognostic information. It can serve as an indicator of the severity and extent of kidney injury and help predict the likelihood of adverse outcomes or complications. This information is crucial for patient management, including determining appropriate therapies, monitoring disease progression, and assessing the effectiveness of interventions aimed at preserving renal function.

### PAX2 Amplification in Cats.

Oligonucleotides were designed to feline PAX2 in two different regions with substantial sequence identity to that of the human PAX2 marker. These oligonucleotides are shown in Table 12 as SEQ ID 50, SEQ ID 51 and SEQ ID 52 (assay PAX2-A), and SEQ ID 57, SEQ ID 58 and SEQ ID 59 (assay PAX2-B).

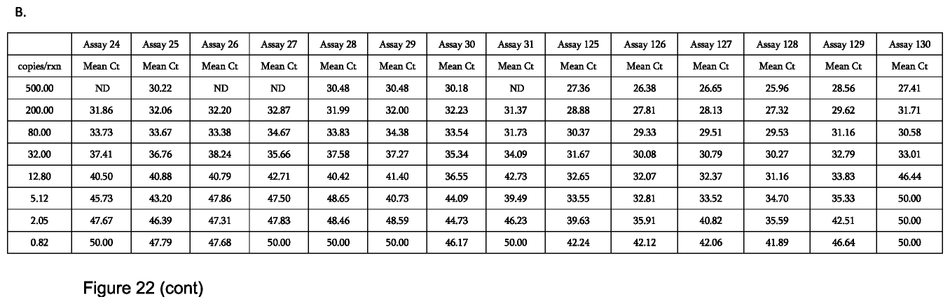

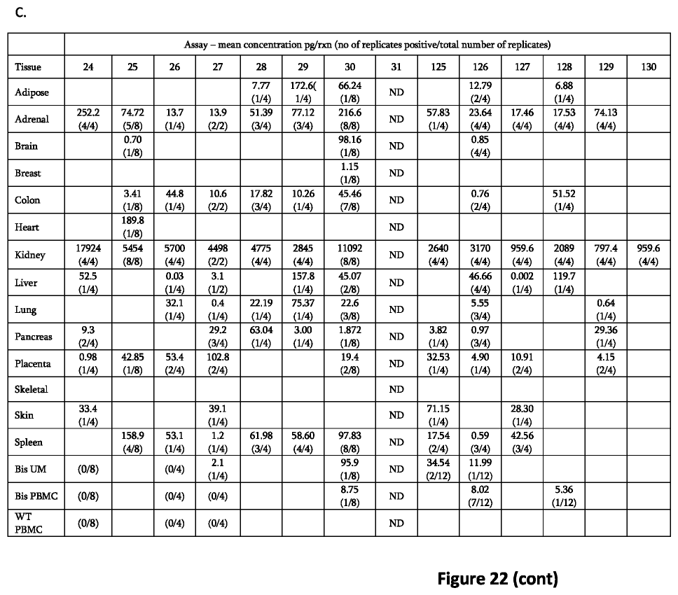

The alignment of the relevant sequences is shown in FIG. 22. The subsequent dPCR assay was tested on bisulphite converted cfDNA extracted from 1 mL urine from 5 cats, 2 healthy and 3 with known CKD. dPCR reactions comprised 10 μL QIAcuity probe PCR mix (Qiagen, Cat #250103), 800 nM of each Forward and Reverse primers, 400 nM fluorescently labelled hydrolysis probe, and template DNA made up to 40 μL final volume, and were cycled as follows: 95° C., 2 mins; [95° C., 15 secs; 60° C., 30 secs with acquisition]×40 on the QIAcuity 4 plate digital PCR system (Qiagen).

The results demonstrate that there is a marked increase in the amount of PAX2 detected in the urine of those cats with CKD relative to healthy cats, for both PAX2 assays see FIG. 20. The results of this experiment indicate the utility of using differential methylated regions to detect kidney-specific cfDNA in different animal species.

It will be appreciated by those skilled in the art that the present disclosure may be embodied in many other forms.

1. A method of diagnosing kidney damage in a subject, the method comprising detecting at least one kidney differentially methylated region in cfDNA wherein the cfDNA is obtained from a biological sample of the subject, and wherein the presence of the at least one kidney differentially methylated region in the cfDNA is indicative of kidney damage.

2. A method of detecting kidney damage in a subject, the method comprising:
a) obtaining a biological sample comprising cfDNA from the subject; and
b) detecting at least one kidney differentially methylated region in the cfDNA,
wherein the presence of the at least one kidney-specific methylation site in the cfDNA is indicative of organ damage.

3. The method of claim 1 wherein the method comprises detecting an increase in the level of the at least one kidney differentially methylated region relative to a reference level.

4. The method of claim 1 wherein the method comprises detecting an increase in the level of the at least one kidney differentially methylated region over time.

5. The method of claim 1 wherein the method comprises detecting cfDNA methylation status at more than one kidney differentially methylated region.

6. The method of claim 5 wherein the methylation status is determined at more than one kidney differentially methylated region using a multiplex assay.

7. The method of claim 1 wherein the methylation status is determined by a method that does not involve DNA sequencing.

8. The method of claim 1 wherein the methylation status is determined by treating the cfDNA with bisulfite and amplifying the at least one kidney differentially methylated region using polymerase chain reaction (PCR).

9. The method of claim 8 wherein the PCR is digital PCR (dPCR), digital droplet PCR (ddPCR) or quantitative PCR (qPCR).

10. The method of claim 1 wherein the subject and the kidney are autologous.

11. The method of claim 1 wherein the kidney damage is associated with acute kidney injury, chronic kidney disease or kidney transplant rejection or renal replacement therapy.

12. The method of claim 1 wherein the kidney damage is associated with chemotherapy or radiotherapy.

13. The method of claim 1 wherein the biological sample is urine.

14. The method of claim 1 wherein the at least one kidney differentially methylated region is located at one or more loci selected from the group consisting of GRAMD1B, DDC, MAST4, MCF2L, PAX2, chr12-122277360 (CLIP1), chr17-35303285, DEF6, EMX1, HPD, PDE4D and SPAG5.

15. The method of claim 5 wherein the at least one kidney differentially methylated region is located at one or more loci selected from the group consisting of GRAMD1B, DDC, MAST4, MCF2L and PAX2.

16. The method of claim 1 wherein the at least one kidney differentially methylated region comprises a sequence having at least 90% identity to any one or more of SEQ ID NO. 1, SEQ ID NO. 2, SEQ ID NO. 8, SEQ ID NO. 9, SEQ ID NO. 15, SEQ ID NO. 16, SEQ ID NO. 22, SEQ ID NO. 23, SEQ ID NO. 29 or SEQ ID NO. 30.

17. The method of claim 1 wherein the method specifically detects damage to a defined tissue or cell-type of the kidney.

18. The method of claim 17 wherein the defined cell-type is renal proximal tubule epithelial cells.

19. The method of claim 17 wherein the at least one kidney differentially methylated regions are located at at least one of MAST4 and DDC.

20. The method of claim 1 wherein the method further comprises treating the subject for the kidney damage.


SEQUENCE LISTING (66 sequences)

SEQ ID NO: 1 - DNA, 59 bp, synthetic construct
  source 1..59: mol_type = other DNA
tcagcgcggaccgcagcgcggcccagccccgggcacccgcctcggacgctcgggcgcca

SEQ ID NO: 2 - DNA, 59 bp, synthetic construct
  source 1..59: mol_type = other DNA
tggcgcccgagcgtccgaggcgggtgcccggggctgggccgcgctgcggtccgcgctga

SEQ ID NO: 3 - DNA, 59 bp, synthetic construct
  source 1..59: mol_type = other DNA
ttagcgcggatcgtagcgcggtttagtttcgggtattcgtttcggacgttcgggcgtta

SEQ ID NO: 4 - DNA, 59 bp, synthetic construct
  source 1..59: mol_type = other DNA
taacgcccgaacgtccgaaacgaatacccgaaactaaaccgcgctacgatccgcgctaa

SEQ ID NO: 5 - DNA, 17 bp, synthetic construct
  source 1..17: mol_type = other DNA
ttagcgcggatcgtagc

SEQ ID NO: 6 - DNA, 16 bp, synthetic construct
  source 1..16: mol_type = other DNA
taacgcccgaacgtcc

SEQ ID NO: 7 - DNA, 25 bp, synthetic construct
  source 1..25: mol_type = other DNA
aaacgaatacccgaaactaaaccgc

SEQ ID NO: 8 - DNA, 67 bp, synthetic construct
  source 1..67: mol_type = other DNA
agacgccagcaagcgaggaagcgcagcggaagaaaaacaagcgggcgcgcgaggggagccccaggag

SEQ ID NO: 9 - DNA, 67 bp, synthetic construct
  source 1..67: mol_type = other DNA
ctcctggggctcccctcgcgcgcccgcttgtttttcttccgctgcgcttcctcgcttgctggcgtct

SEQ ID NO: 10 - DNA, 67 bp, synthetic construct
  source 1..67: mol_type = other DNA
agacgttagtaagcgaggaagcgtagcggaagaaaaataagcgggcgcgcgaggggagttttaggag

SEQ ID NO: 11 - DNA, 67 bp, synthetic construct
  source 1..67: mol_type = other DNA
ctcctaaaactcccctcgcgcgcccgcttatttttcttccgctacgcttcctcgcttactaacgtct

SEQ ID NO: 12 - DNA, 22 bp, synthetic construct
  source 1..22: mol_type = other DNA
agacgttagtaagcgaggaagc

SEQ ID NO: 13 - DNA, 19 bp, synthetic construct
  source 1..19: mol_type = other DNA
ctcctaaaactcccctcgc

SEQ ID NO: 14 - DNA, 23 bp, synthetic construct
  source 1..23: mol_type = other DNA
cccgcttatttttcttccgctac

SEQ ID NO: 15 - DNA, 84 bp, synthetic construct
  source 1..84: mol_type = other DNA
cgtgccttgctcattctaagcccgcggatacagcagggcccccatgttgcaggtgctggggaggacctgcggaaacgcggcgcc

SEQ ID NO: 16 - DNA, 88 bp, synthetic construct
  source 1..88: mol_type = other DNA
ggcgccgcgtttccgcaggtcctccccagcacctgcaacatgggggccctgctgtatccgcgggcttagaatgagcaaggcacgggcc

SEQ ID NO: 17 - DNA, 84 bp, synthetic construct
  source 1..84: mol_type = other DNA
cgtgttttgtttattttaagttcgcggatatagtagggtttttatgttgtaggtgttggggaggatttgcggaaacgcggcgtt

SEQ ID NO: 18 - DNA, 84 bp, synthetic construct
  source 1..84: mol_type = other DNA
aacgccgcgtttccgcaaatcctccccaacacctacaacataaaaaccctactatatccgcgaacttaaaataaacaaaacacg

SEQ ID NO: 19 - DNA, 25 bp, synthetic construct
  source 1..25: mol_type = other DNA
cgtgttttgtttattttaagttcgc

SEQ ID NO: 20 - DNA, 15 bp, synthetic construct
  source 1..15: mol_type = other DNA
aacgccgcgtttccg

SEQ ID NO: 21 - DNA, 31 bp, synthetic construct
  source 1..31: mol_type = other DNA
cctacaacataaaaaccctactatatccgcg

SEQ ID NO: 22 - DNA, 67 bp, synthetic construct
  source 1..67: mol_type = other DNA
gacggatccattccgggctccggggagggggagcggcggcccaggctgcggggctgtcccggcaccg

SEQ ID NO: 23 - DNA, 67 bp, synthetic construct
  source 1..67: mol_type = other DNA
cggtgccgggacagccccgcagcctgggccgccgctccccctccccggagcccggaatggatccgtc

SEQ ID NO: 24 - DNA, 67 bp, synthetic construct
  source 1..67: mol_type = other DNA
gacggatttatttcgggtttcggggagggggagcggcggtttaggttgcggggttgtttcggtatcg

SEQ ID NO: 25 - DNA, 67 bp, synthetic construct
  source 1..67: mol_type = other DNA
cgataccgaaacaaccccgcaacctaaaccgccgctccccctccccgaaacccgaaataaatccgtc

SEQ ID NO: 26 - DNA, 21 bp, synthetic construct
  source 1..21: mol_type = other DNA
gacggatttatttcgggtttc

SEQ ID NO: 27 - DNA, 18 bp, synthetic construct
  source 1..18: mol_type = other DNA
cgataccgaaacaacccc

SEQ ID NO: 28 - DNA, 18 bp, synthetic construct
  source 1..18: mol_type = other DNA
ctaaaccgccgctccccc

SEQ ID NO: 29 - DNA, 74 bp, synthetic construct
  source 1..74: mol_type = other DNA
cgctgccgtggccccctccccgcctccgccgcgccccctccgcactcgcacggccccacccgcaggcgcccccc

SEQ ID NO: 30 - DNA, 74 bp, synthetic construct
  source 1..74: mol_type = other DNA
ggggggcgcctgcgggtggggccgtgcgagtgcggagggggcgcggcggaggcggggagggggccacggcagcg

SEQ ID NO: 31 - DNA, 74 bp, synthetic construct
  source 1..74: mol_type = other DNA
cgttgtcgtggtttttttttcgttttcgtcgcgtttttttcgtattcgtacggttttattcgtaggcgtttttc

SEQ ID NO: 32 - DNA, 74 bp, synthetic construct
  source 1..74: mol_type = other DNA
gaaaaacgcctacgaataaaaccgtacgaatacgaaaaaaacgcgacgaaaacgaaaaaaaaaccacgacaacg

SEQ ID NO: 33 - DNA, 21 bp, synthetic construct
  source 1..21: mol_type = other DNA
cgttgtcgtggtttttttttc

SEQ ID NO: 34 - DNA, 23 bp, synthetic construct
  source 1..23: mol_type = other DNA
gaaaaacgcctacgaataaaacc

SEQ ID NO: 35 - DNA, 24 bp, synthetic construct
  source 1..24: mol_type = other DNA
tacgaatacgaaaaaaacgcgacg

SEQ ID NO: 36 - DNA, 134 bp, synthetic construct
  source 1..134: mol_type = other DNA
ctcagccaatgggacctgctcctcccttgaaggttgcagaggccacagcctggtgggaaagatgaccaccacccagcacacagtggcagacacaggttcacagtccagaagacttgctgagcctcctccatcac

SEQ ID NO: 37 - DNA, 134 bp, synthetic construct
  source 1..134: mol_type = other DNA
gtgatggaggaggctcagcaagtcttctggactgtgaacctgtgtctgccactgtgtgctgggtggtggtcatctttcccaccaggctgtggcctctgcaaccttcaagggaggagcaggtcccattggctgag

SEQ ID NO: 38 - DNA, 19 bp, synthetic construct
  source 1..19: mol_type = other DNA
ggaggaggctcagcaagtc

SEQ ID NO: 39 - DNA, 18 bp, synthetic construct
  source 1..18: mol_type = other DNA
ctcagccaatgggacctg

SEQ ID NO: 40 - DNA, 23 bp, synthetic construct
  source 1..23: mol_type = other DNA
acctgtgtctgccactgtgtgct

SEQ ID NO: 41 - DNA, 89 bp, synthetic construct
  source 1..89: mol_type = other DNA
caaaataaaatacaaaacaaacctaatccccaaacaaaacctatattaccactaaacctccattcaactaaccaaaaaacaaaaactcc

SEQ ID NO: 42 - DNA, 89 bp, synthetic construct
  source 1..89: mol_type = other DNA
ggagtttttgttttttggttagttgaatggaggtttagtggtaatataggttttgtttggggattaggtttgttttgtattttattttg

SEQ ID NO: 43 - DNA, 25 bp, synthetic construct
  source 1..25: mol_type = other DNA
ggagtttttgttttttggttagttg

SEQ ID NO: 44 - DNA, 29 bp, synthetic construct
  source 1..29: mol_type = other DNA
caaaataaaatacaaaacaaacctaatcc

SEQ ID NO: 45 - DNA, 34 bp, synthetic construct
  source 1..34: mol_type = other DNA
atggaggtttagtggtaatataggttttgtttgg

SEQ ID NO: 46 - DNA, 65 bp, synthetic construct
  source 1..65: mol_type = other DNA
acaatcagcgcggactgcagcgcggcccagccccggagcagcccgcctcggacgctccggcgcct

SEQ ID NO: 47 - DNA, 65 bp, synthetic construct
  source 1..65: mol_type = other DNA
aggcgccggagcgtccgaggcgggctgctccggggctgggccgcgctgcagtccgcgctgattgt

SEQ ID NO: 48 - DNA, 65 bp, synthetic construct
  source 1..65: mol_type = other DNA
ataattagcgcggattgtagcgcggtttagtttcggagtagttcgtttcggacgtttcggcgttt

SEQ ID NO: 49 - DNA, 65 bp, synthetic construct
  source 1..65: mol_type = other DNA
aaacgccgaaacgtccgaaacgaactactccgaaactaaaccgcgctacaatccgcgctaattat

SEQ ID NO: 50 - DNA, 21 bp, synthetic construct
  source 1..21: mol_type = other DNA
ataattagcgcggattgtagc

SEQ ID NO: 51 - DNA, 16 bp, synthetic construct
  source 1..16: mol_type = other DNA
aaacgccgaaacgtcc

SEQ ID NO: 52 - DNA, 27 bp, synthetic construct
  source 1..27: mol_type = other DNA
aaacgaactactccgaaactaaaccgc

SEQ ID NO: 53 - DNA, 66 bp, synthetic construct
  source 1..66: mol_type = other DNA
ctgcagcgcggcccagccccggagcagcccgcctcggacgctccggcgcctggaggcttcgctggt

SEQ ID NO: 54 - DNA, 66 bp, synthetic construct
  source 1..66: mol_type = other DNA
accagcgaagcctccaggcgccggagcgtccgaggcgggctgctccggggctgggccgcgctgcag

SEQ ID NO: 55 - DNA, 66 bp, synthetic construct
  source 1..66: mol_type = other DNA
ttgtagcgcggtttagtttcggagtagttcgtttcggacgtttcggcgtttggaggtttcgttggt

SEQ ID NO: 56 - DNA, 66 bp, synthetic construct
  source 1..66: mol_type = other DNA
accaacgaaacctccaaacgccgaaacgtccgaaacgaactactccgaaactaaaccgcgctacaa

SEQ ID NO: 57 - DNA, 20 bp, synthetic construct
  source 1..20: mol_type = other DNA
ttgtagcgcggtttagtttc

SEQ ID NO: 58 - DNA, 19 bp, synthetic construct
  source 1..19: mol_type = other DNA
accaacgaaacctccaaac

SEQ ID NO: 59 - DNA, 26 bp, synthetic construct
  source 1..26: mol_type = other DNA
ccgaaacgtccgaaacgaactactcc

SEQ ID NO: 60 - DNA, 64 bp, synthetic construct
  source 1..64: mol_type = other DNA
ccgcagcgcggcccagcccgggagcagcccgcctcggacgctccggcgccgggaggcttcgctg

SEQ ID NO: 61 - DNA, 64 bp, synthetic construct
  source 1..64: mol_type = other DNA
cagcgaagcctcccggcgccggagcgtccgaggcgggctgctcccgggctgggccgcgctgcgg

SEQ ID NO: 62 - DNA, 64 bp, synthetic construct
  source 1..64: mol_type = other DNA
tcgtagcgcggtttagttcgggagtagttcgtttcggacgtttcggcgtcgggaggtttcgttg

SEQ ID NO: 63 - DNA, 64 bp, synthetic construct
  source 1..64: mol_type = other DNA
caacgaaacctcccgacgccgaaacgtccgaaacgaactactcccgaactaaaccgcgctacga

SEQ ID NO: 64 - DNA, 19 bp, synthetic construct
  source 1..19: mol_type = other DNA
tcgtagcgcggtttagttc

SEQ ID NO: 65 - DNA, 17 bp, synthetic construct
  source 1..17: mol_type = other DNA
caacgaaacctcccgac

SEQ ID NO: 66 - DNA, 26 bp, synthetic construct
  source 1..26: mol_type = other DNA
ccgaaacgtccgaaacgaactactcc